SEQUENCE LISTING (102 sequences)

SEQ ID NO: 1 - protein, 179 aa, Artificial Sequence
  amino acids of human CD94
MAVFKTTLWRLISGTLGIICLSLMSTLGILLKNSFTKLSIEPAFTPGPNIELQKDSDCCSCQEKWVGYRCNCYFISSEQKTWNESRHLCASQKSSLLQLQNTDELDFMSSSQQFYWIGLSYSEEHTAWLWENGSALSQYLFPSFETFNTKNCIAYNPNGNALDESCEDKNRYICKQQLI

SEQ ID NO: 2 - DNA, 537 bp, Artificial Sequence
  nucleic acid of human CD94
atggcagtgtttaagaccactctgtggaggttaatttctgggaccttagggataatatgcctttcgttgatgtctacgttgggaattttgttgaaaaattcttttactaaactgagtattgagccagcatttactccaggacccaacatagaactccagaaagactctgactgctgttcttgccaagaaaaatgggttgggtaccggtgcaactgttacttcatttccagtgaacagaaaacttggaacgaaagtcggcatctctgtgcttctcagaaatccagcctgcttcagcttcaaaacacagatgaactggattttatgagctccagtcaacaattttactggattggactctcttacagtgaggagcacaccgcctggttgtgggagaatggctctgcactctcccagtatctatttccatcatttgaaacttttaatacaaagaactgcatagcgtataatccaaatggaaatgctttagatgaatcctgtgaagataaaaatcgttatatctgtaagcaacagctcatt

SEQ ID NO: 3 - protein, 148 aa, Artificial Sequence
  amino acids of CD94 extracellular domain
KNSFTKLSIEPAFTPGPNIELQKDSDCCSCQEKWVGYRCNCYFISSEQKTWNESRHLCASQKSSLLQLQNTDELDFMSSSQQFYWIGLSYSEEHTAWLWENGSALSQYLFPSFETFNTKNCIAYNPNGNALDESCEDKNRYICKQQLI

SEQ ID NO: 4 - DNA, 444 bp, Artificial Sequence
  bases of CD94 extracellular domain
aaaaattcttttactaaactgagtattgagccagcatttactccaggacccaacatagaactccagaaagactctgactgctgttcttgccaagaaaaatgggttgggtaccggtgcaactgttacttcatttccagtgaacagaaaacttggaacgaaagtcggcatctctgtgcttctcagaaatccagcctgcttcagcttcaaaacacagatgaactggattttatgagctccagtcaacaattttactggattggactctcttacagtgaggagcacaccgcctggttgtgggagaatggctctgcactctcccagtatctatttccatcatttgaaacttttaatacaaagaactgcatagcgtataatccaaatggaaatgctttagatgaatcctgtgaagataaaaatcgttatatctgtaagcaacagctcatt

SEQ ID NO: 5 - protein, 140 aa, Artificial Sequence
  amino acids of NKG2A extracellular domain
PSTLIQRHNNSSLNTRTQKARHCGHCPEEWITYSNSCYYIGKERRTWEESLLACTSKNSSLLSIDNEEEMKFLSIISPSSWIGVFRNSSHHPWVTMNGLAFKHEIKDSDNAELNCAVLQVNRLKSAQCGSSIIYHCKHKL

SEQ ID NO: 6 - DNA, 420 bp, Artificial Sequence
  bases of NKG2A extracellular domain
ccctctacattaatacagaggcacaacaattcttccctgaatacaagaactcagaaagcacgtcattgtggccattgtcctgaggagtggattacatattccaacagttgttactacattggtaaggaaagaagaacttgggaagagagtttgctggcctgtacttcgaagaactccagtctgctttctatagataatgaagaagaaatgaaatttctgtccatcatttcaccatcctcatggattggtgtgtttcgtaacagcagtcatcatccatgggtgacaatgaatggtttggctttcaaacatgagataaaagactcagataatgctgaacttaactgtgcagtgctacaagtaaatcgacttaaatcagcccagtgtggatcttcaataatatatcattgtaagcataagctt

SEQ ID NO: 7 - protein, 227 aa, Artificial Sequence [sequence omitted]

SEQ ID NO: 8 - DNA, 681 bp, Artificial Sequence
  bases of huFc(knob)
gacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 9 - protein, 227 aa, Artificial Sequence
  amino acids of huFc(hole)
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 10 - DNA, 681 bp, Artificial Sequence
  bases of huFc(hole)
gacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgagctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 11 - protein, 5 aa, Artificial Sequence
  2B12F1-HCDR1
SYAMS

SEQ ID NO: 12 - protein, 17 aa, Artificial Sequence
  2B12F1-HCDR2
AISGSGGSTYYADSVKG

SEQ ID NO: 13 - protein, 12 aa, Artificial Sequence
  2B1-HCDR3
SSTRWTSSTFDY

SEQ ID NO: 14 - protein, 11 aa, Artificial Sequence
  2B12F1-LCDR1
RASQSISSWLA

SEQ ID NO: 15 - protein, 7 aa, Artificial Sequence
  2B12F1-LCDR2
DASSLES

SEQ ID NO: 16 - protein, 9 aa, Artificial Sequence
  2B1-LCDR3
QQYYFYPYT

SEQ ID NO: 17 - protein, 12 aa, Artificial Sequence
  2F1-HCDR3
YEGRYVLGAMDY

SEQ ID NO: 18 - protein, 9 aa, Artificial Sequence
  2F1-LCDR3
QQYFSYPLT

SEQ ID NO: 19 - protein, 121 aa, Artificial Sequence
  amino acids of 2B1-VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSSTRWTSSTFDYWGQGTLVTVSS

SEQ ID NO: 20 - DNA, 363 bp, Artificial Sequence
  bases of 2B1-VH
gaggtgcaattgctggagtctgggggaggcttggtacagcctggggggtccctgagactctcctgtgcagcctccggattcacctttagcagttatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcagatgaacagcctgagagccgaggacacggccgtatattactgtgcgaaatcttctactcgttggacttcttctactttcgactactggggccaaggaaccctggtcaccgtctcgagt

SEQ ID NO: 21 - protein, 108 aa, Artificial Sequence
  amino acids of 2B1-VL
DIQMTQSPSTLSASVGDRVTITCRASQSISSWLAWYQQKPGKAPKLLIYDASSLESGVPSRFSGSGSGTEFTLTISSLQPDDFATYYCQQYYFYPYTFGQGTKVEIKR

SEQ ID NO: 22 - DNA, 324 bp, Artificial Sequence
  bases of 2B1-VL
gacatccagatgacccagtctccttccaccctgtctgcatctgtaggagaccgtgtcaccatcacttgccgtgccagtcagagtattagtagctggttggcctggtatcagcagaaaccagggaaagcccctaagctcctgatctatgatgcctccagtttggaaagtggggtcccatcacgtttcagcggcagtggatccgggacagaattcactctcaccatcagcagcttgcagcctgatgattttgcaacttattactgccaacagtactacttctacccatacacgtttggccagggcaccaaagtcgagatcaagcgt

SEQ ID NO: 23 - protein, 244 aa, Artificial Sequence
  amino acids of 2B1-scFv
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSSTRWTSSTFDYWGQGTLVTVSSGGGGSGGGGSGGGGSDIQMTQSPSTLSASVGDRVTITCRASQSISSWLAWYQQKPGKAPKLLIYDASSLESGVPSRFSGSGSGTEFTLTISSLQPDDFATYYCQQYYFYPYTFGQGTKVEIKR

SEQ ID NO: 24 - protein, 121 aa, Artificial Sequence
  amino acids of 2F1-VH
EVQLLESGGGLVQPGGSMRLSCAASGFTFSSYAMSWVRQAPGKGLEWVSAISGSGGSTYYADSVKGRFTISRDNSKNTLYLQMNSLRAEDTAVYYCAKYEGRYVLGAMDYWGQGTLVTVSS

SEQ ID NO: 25 - DNA, 363 bp, Artificial Sequence
  bases of 2F1-VH
gaggtgcaattgctggagtctgggggaggcttggtacagcctggggggtccatgagactctcctgtgcagcctccggattcacctttagcagttatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcagatgaacagcctgagagccgaggacacggccgtatattactgtgcgaaatacgaaggtcgttacgttctgggtgcaatggactactggggccaaggaaccctggtcaccgtctcgagt

SEQ ID NO: 26 - protein, 108 aa, Artificial Sequence
  amino acids of 2F1-VL
DIQMTQSPSTLSASVGDRVTITCRASQSISSWLAWYQQKPGKAPKLLIYDASSLESGVPSRFSGSGSGTEFTLTISSLQPDDFATYYCQQYFSYPLTFGQGTKVEIKR

SEQ ID NO: 27 - DNA, 324 bp, Artificial Sequence
  bases of 2F1-VL
gacatccagatgacccagtctccttccaccctgtctgcatctgtaggagaccgtgtcaccatcacttgccgtgccagtcagagtattagtagctggttggcctggtatcagcagaaaccagggaaagcccctaagctcctgatctatgatgcctccagtttggaaagtggggtcccatcacgtttcagcggcagtggatccgggacagaattcactctcaccatcagcagcttgcagcctgatgattttgcaacttattactgccaacagtacttctcttacccactgacgtttggccagggcaccaaagtcgagatcaagcgt

SEQ ID NO: 28 - protein, 244 aa, Artificial Sequence
  amino acids of 2F1-scFv
EVQLLESGGGLVQPGGSMRLSCAASGFTFSSYAMSWVRQAPGKGLEWVSAISGSGGSTYYADSVKGRFTISRDNSKNTLYLQMNSLRAEDTAVYYCAKYEGRYVLGAMDYWGQGTLVTVSSGGGGSGGGGSGGGGSDIQMTQSPSTLSASVGDRVTITCRASQSISSWLAWYQQKPGKAPKLLIYDASSLESGVPSRFSGSGSGTEFTLTISSLQPDDFATYYCQQYFSYPLTFGQGTKVEIKR

SEQ ID NO: 29 - protein, 21 aa, Artificial Sequence
  amino acids of CD8 signal peptide
MALPVTALLLPLALLLHAARP

SEQ ID NO: 30 - DNA, 63 bp, Artificial Sequence
  nucleic acid of CD8 signal peptide
atggccttaccagtgaccgccttgctcctgccgctggccttgctgctccacgccgccaggccg

SEQ ID NO: 31 - protein, 45 aa, Artificial Sequence
  amino acids of CD8 hinge region
TTTPAPRPPTPAPTIASQPLSLRPEACRPAAGGAVHTRGLDFACD

SEQ ID NO: 32 - DNA, 135 bp, Artificial Sequence
  nucleic acid of CD8 hinge region
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 33 - protein, 24 aa, Artificial Sequence
  amino acids of CD8 transmembrane domain
IYIWAPLAGTCGVLLLSLVITLYC

SEQ ID NO: 34 - DNA, 72 bp, Artificial Sequence
  nucleic acid of CD8 transmembrane domain
atctacatctgggcgcccttggccgggacttgtggggtccttctcctgtcactggttatcaccctttactgc

SEQ ID NO: 35 - protein, 27 aa, Artificial Sequence
  amino acids of CD28 transmembrane domain
FWVLVVVGGVLACYSLLVTVAFIIFWV

SEQ ID NO: 36 - DNA, 81 bp, Artificial Sequence
  nucleic acid of CD28 transmembrane domain
ttttgggtgctggtggtggttggtggagtcctggcttgctatagcttgctagtaacagtggcctttattattttctgggtg

SEQ ID NO: 37 - protein, 41 aa, Artificial Sequence
  amino acids of CD28 intracellular domain
RSKRSRLLHSDYMNMTPRRPGPTRKHYQPYAPPRDFAAYRS

SEQ ID NO: 38 - DNA, 123 bp, Artificial Sequence
  nucleic acid of CD28 intracellular domain
aggagtaagaggagcaggctcctgcacagtgactacatgaacatgactccccgccgccccgggccaacccgcaagcattaccagccctatgccccaccacgcgacttcgcagcctatcgctcc

SEQ ID NO: 39 - protein, 42 aa, Artificial Sequence
  amino acids of CD137 intracellular domain
KRGRKKLLYIFKQPFMRPVQTTQEEDGCSCRFPEEEEGGCEL

SEQ ID NO: 40 - DNA, 126 bp, Artificial Sequence
  nucleic acid of CD137 intracellular domain
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 41 - protein, 113 aa, Artificial Sequence
  amino acids of CD3
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 42 - DNA, 339 bp, Artificial Sequence
  nucleic acid of CD3
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 43 - protein, 470 aa, Artificial Sequence
  amino acids of 2B1-28Z
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSSTRWTSSTFDYWGQGTLVTVSSGGGGSGGGGSGGGGSDIQMTQSPSTLSASVGDRVTITCRASQSISSWLAWYQQKPGKAPKLLIYDASSLESGVPSRFSGSGSGTEFTLTISSLQPDDFATYYCQQYYFYPYTFGQGTKVEIKRTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 44 - protein, 468 aa, Artificial Sequence
  amino acids of 2B1-BBZ
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSSTRWTSSTFDYWGQGTLVTVSSGGGGSGGGGSGGGGSDIQMTQSPSTLSASVGDRVTITCRASQSISSWLAWYQQKPGKAPKLLIYDASSLESGVPSRFSGSGSGTEFTLTISSLQPDDFATYYCQQYYFYPYTFGQGTKVEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 45 - protein, 470 aa, Artificial Sequence
  amino acids of 2F1-28Z
EVQLLESGGGLVQPGGSMRLSCAASGFTFSSYAMSWVRQAPGKGLEWVSAISGSGGSTYYADSVKGRFTISRDNSKNTLYLQMNSLRAEDTAVYYCAKYEGRYVLGAMDYWGQGTLVTVSSGGGGSGGGGSGGGGSDIQMTQSPSTLSASVGDRVTITCRASQSISSWLAWYQQKPGKAPKLLIYDASSLESGVPSRFSGSGSGTEFTLTISSLQPDDFATYYCQQYFSYPLTFGQGTKVEIKRTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 46 - protein, 468 aa, Artificial Sequence
  amino acids of 2F1-BBZ
EVQLLESGGGLVQPGGSMRLSCAASGFTFSSYAMSWVRQAPGKGLEWVSAISGSGGSTYYADSVKGRFTISRDNSKNTLYLQMNSLRAEDTAVYYCAKYEGRYVLGAMDYWGQGTLVTVSSGGGGSGGGGSGGGGSDIQMTQSPSTLSASVGDRVTITCRASQSISSWLAWYQQKPGKAPKLLIYDASSLESGVPSRFSGSGSGTEFTLTISSLQPDDFATYYCQQYFSYPLTFGQGTKVEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 47 - DNA, 20 bp, Artificial Sequence
  TRAC-gRNA1
agagtctctcagctggtaca

SEQ ID NO: 48 - DNA, 20 bp, Artificial Sequence
  TRAC-gRNA2
tctctcagctggtacacggc

SEQ ID NO: 49 - DNA, 20 bp, Artificial Sequence
  TRAC-gRNA3
gagaatcaaaatcggtgaat

SEQ ID NO: 50 - DNA, 20 bp, Artificial Sequence
  TRAC-gRNA4
ctctcagctggtacacggca

SEQ ID NO: 51 - DNA, 17 bp, Artificial Sequence
  TRAC-gRNA5
gtctctcagctggtaca

SEQ ID NO: 52 - DNA, 18 bp, Artificial Sequence
  TRAC-gRNA6
agtctctcagctggtaca

SEQ ID NO: 53 - DNA, 22 bp, Artificial Sequence
  TRAC-gRNA7
ttagagtctctcagctggtaca

SEQ ID NO: 54 - DNA, 23 bp, Artificial Sequence
  TRAC-gRNA8
tttagagtctctcagctggtaca

SEQ ID NO: 55 - DNA, 20 bp, Artificial Sequence
  B2M-gRNA1
gagtagcgcgagcacagcta

SEQ ID NO: 56 - DNA, 20 bp, Artificial Sequence
  B2M-gRNA2
ggccacggagcgagacatct

SEQ ID NO: 57 - DNA, 20 bp, Artificial Sequence
  B2M-gRNA3
cgcgagcacagctaaggcca

SEQ ID NO: 58 - DNA, 20 bp, Artificial Sequence
  B2M-gRNA4
aagtggaggcgtcgcgctgg

SEQ ID NO: 59 - DNA, 20 bp, Artificial Sequence
  CD94 gRNA-1
cctttcgttgatgtctacgt

SEQ ID NO: 60 - DNA, 20 bp, Artificial Sequence
  CD94 gRNA-2
ccaacgtagacatcaacgaa

SEQ ID NO: 61 - DNA, 20 bp, Artificial Sequence
  CD94 gRNA-3
gattggactctcttacagtg

SEQ ID NO: 62 - DNA, 20 bp, Artificial Sequence
  CD94 gRNA-4
cgaaaggcatattatcccta

SEQ ID NO: 63 - DNA, 20 bp, Artificial Sequence
  CD94 gRNA-5
attgagccagcatttactcc

SEQ ID NO: 64 - DNA, 20 bp, Artificial Sequence
  CD94 gRNA-6
cagtgtttaagaccactctg

SEQ ID NO: 65 - DNA, 20 bp, Artificial Sequence
  CD94 gRNA-7
actctgtggaggttaatttc

SEQ ID NO: 66 - DNA, 19 bp, Artificial Sequence
  CD94 gRNA-8
attggactctcttacagtg

SEQ ID NO: 67 - DNA, 19 bp, Artificial Sequence
  CD94 gRNA-9
gtacccaacccatttttct

SEQ ID NO: 68 - DNA, 19 bp, Artificial Sequence
  CD94 gRNA-10
gtttaagaccactctgtgg

SEQ ID NO: 69 - DNA, 19 bp, Artificial Sequence
  CD94 gRNA-11
ttccaagttttctgttcac

SEQ ID NO: 70 - DNA, 19 bp, Artificial Sequence
  CD94 gRNA-12
gaaaacttggaacgaaagt

SEQ ID NO: 71 - DNA, 19 bp, Artificial Sequence
  CD94 gRNA-13
gagccattctcccacaacc

SEQ ID NO: 72 - protein, 247 aa, Artificial Sequence
  amino acids of anti-CLDN18A2 scFv
QVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSSGGGGSGGGGSGGGGSDIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKR

SEQ ID NO: 73 - DNA, 741 bp, Artificial Sequence
  nucleic acid of anti-CLDN18A2 scFv
caggtgcagctgcaggagagcggccccggcctgatcaagcccagccagaccctgagcctgacctgcaccgtgagcggcggcagcatcagcagcggctacaactggcactggatccggcagccccccggcaagggcctggagtggatcggctacatccactacaccggcagcaccaactacaaccccgccctgcggagccgggtgaccatcagcgtggacaccagcaagaaccagttcagcctgaagctgagcagcgtgaccgccgccgacaccgccatctactactgcgcccggatctacaacggcaacagcttcccctactggggccagggcaccaccgtgaccgtgagcagcggtggaggcggttcaggcggaggtggttctggcggtggcggatcggacatcgtgatgacccagagccccgacagcctggccgtgagcctgggcgagcgggccaccatcaactgcaagagcagccagagcctgttcaacagcggcaaccagaagaactacctgacctggtaccagcagaagcccggccagccccccaagctgctgatctactgggccagcacccgggagagcggcgtgcccgaccggttcagcggcagcggcagcggcaccgacttcaccctgaccatcagcagcctgcaggccgaggacgtggccgtgtactactgccagaacgcctacagcttcccctacaccttcggcggcggcaccaagctggagatcaagcgg

SEQ ID NO: 74 - protein, 473 aa, Artificial Sequence
  CLDN18A2-CAR1
QVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSSGGGGSGGGGSGGGGSDIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKRTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 75 - protein, 261 aa, Artificial Sequence
  amino acids of human CLDN18A2
MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 76 - DNA, 783 bp, Artificial Sequence
  nucleic acid of human CLDN18A2
atggccgtgactgcctgtcagggcttggggttcgtggtttcactgattgggattgcgggcatcattgctgccacctgcatggaccagtggagcacccaagacttgtacaacaaccccgtaacagctgttttcaactaccaggggctgtggcgctcctgtgtccgagagagctctggcttcaccgagtgccggggctacttcaccctgctggggctgccagccatgctgcaggcagtgcgagccctgatgatcgtaggcatcgtcctgggtgccattggcctcctggtatccatctttgccctgaaatgcatccgcattggcagcatggaggactctgccaaagccaacatgacactgacctccgggatcatgttcattgtctcaggtctttgtgcaattgctggagtgtctgtgtttgccaacatgctggtgactaacttctggatgtccacagctaacatgtacaccggcatgggtgggatggtgcagactgttcagaccaggtacacatttggtgcggctctgttcgtgggctgggtcgctggaggcctcacactaattgggggtgtgatgatgtgcatcgcctgccggggcctggcaccagaagaaaccaactacaaagccgtttcttatcatgcctcaggccacagtgttgcctacaagcctggaggcttcaaggccagcactggctttgggtccaacaccaaaaacaagaagatatacgatggaggtgcccgcacagaggacgaggtacaatcttatccttccaagcacgactatgtg

SEQ ID NO: 77 - protein, 244 aa, Artificial Sequence
  amino acids of anti-BCMA scFv
EVQLLESGGGLVQPGGSLRLSCAASGFTFGGNAMSWVRQAPGKGLEWVSAISGNGGSTFYADSVKGRFTISRDNSKNTLYLQMNSLRAEDTAVYYCAKVRPFWGTFDYWGQGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYFNPPEYTFGQGTKVEIKR

SEQ ID NO: 78 - DNA, 732 bp, Artificial Sequence
  nucleic acid of anti-BCMA scFv
gaggtgcaattgctggagtctgggggaggcttggtacagcctggggggtccctgagactctcctgtgcagcctccggattcacctttggcggtaatgccatgtcctgggtccgccaggctccagggaaggggctggagtgggtctcagcaattagtggtaatggtggtagtacattctacgcagactccgtgaagggccggttcaccatctccagagacaattccaagaacacgctgtatctgcagatgaacagcctgagagccgaggacacggccgtatattactgtgcgaaagttcgtccattctggggtactttcgactactggggccaaggaaccctggtcaccgtctcgagtggtggaggcggttcaggcggaggtggttctggcggtggcggatcggaaatcgtgttaacgcagtctccaggcaccctgtctttgtctccaggggaaagagccaccctctcttgcagggccagtcagagtgttagcagcagctacttagcctggtaccagcagaaacctggccaggctcccaggctcctcatctatggagcatccagcagggccactggcatcccagacaggttcagtggcagtggatccgggacagacttcactctcaccatcagcagactggagcctgaagattttgcagtgtattactgtcagcagtacttcaacccaccagaatacacgttcggccaggggaccaaagtggaaatcaaacgt

SEQ ID NO: 79 - protein, 468 aa, Artificial Sequence
  BCMA-CAR1
EVQLLESGGGLVQPGGSLRLSCAASGFTFGGNAMSWVRQAPGKGLEWVSAISGNGGSTFYADSVKGRFTISRDNSKNTLYLQMNSLRAEDTAVYYCAKVRPFWGTFDYWGQGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYFNPPEYTFGQGTKVEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 80 - protein, 6 aa, Artificial Sequence
  anti-CLDN18A2 antibody-HCDR1
SGYNWH

SEQ ID NO: 81 - protein, 16 aa, Artificial Sequence
  anti-CLDN18A2 antibody-HCDR2
YIHYTGSTNYNPALRS

SEQ ID NO: 82 - protein, 9 aa, Artificial Sequence
  anti-CLDN18A2 antibody-HCDR3
IYNGNSFPY

SEQ ID NO: 83 - protein, 17 aa, Artificial Sequence
  anti-CLDN18A2 antibody-LCDR1
KSSQSLFNSGNQKNYLT

SEQ ID NO: 84 - protein, 7 aa, Artificial Sequence
  anti-CLDN18A2 antibody-LCDR2
WASTRES

SEQ ID NO: 85 - protein, 9 aa, Artificial Sequence
  anti-CLDN18A2 antibody-LCDR3
QNAYSFPYT

SEQ ID NO: 86 - protein, 118 aa, Artificial Sequence
  anti-CLDN18A2 antibody-VH
QVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSS

SEQ ID NO: 87 - protein, 114 aa, Artificial Sequence
  anti-CLDN18A2 antibody-VL
DIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKR

SEQ ID NO: 88 - protein, 474 aa, Artificial Sequence
  CLDN18A2-CAR2
QVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSSGGGGSGGGGSGGGGSDIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKRTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 89 - protein, 429 aa, Artificial Sequence
  CLDN18A2-CAR3
QVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSSGGGGSGGGGSGGGGSDIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKRTTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 90 - protein, 471 aa, Artificial Sequence
  CLDN18A2-CAR4
QVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSSGGGGSGGGGSGGGGSDIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 91 - protein, 5 aa, Artificial Sequence
  anti-BCMA antibody-HCDR1
GNAMS

SEQ ID NO: 92 - protein, 17 aa, Artificial Sequence
  anti-BCMA antibody-HCDR2
AISGNGGSTFYADSVKG

SEQ ID NO: 93 - protein, 10 aa, Artificial Sequence
  anti-BCMA antibody-HCDR3
VRPFWGTFDY

SEQ ID NO: 94 - protein, 12 aa, Artificial Sequence
  anti-BCMA antibody-LCDR1
RASQSVSSSYLA

SEQ ID NO: 95 - protein, 7 aa, Artificial Sequence
  anti-BCMA antibody-LCDR2
GASSRAT

SEQ ID NO: 96 - protein, 9 aa, Artificial Sequence
  anti-BCMA antibody-LCDR3
QQYFNPPEY

SEQ ID NO: 97 - protein, 119 aa, Artificial Sequence
  anti-BCMA antibody-VH
EVQLLESGGGLVQPGGSLRLSCAASGFTFGGNAMSWVRQAPGKGLEWVSAISGNGGSTFYADSVKGRFTISRDNSKNTLYLQMNSLRAEDTAVYYCAKVRPFWGTFDYWGQGTLVTVSS

SEQ ID NO: 98 - protein, 109 aa, Artificial Sequence
  anti-BCMA antibody-VL
EIVLTQSPGTLSLSPGERATLSCRASQSVSSSYLAWYQQKPGQAPRLLIYGASSRATGIPDRFSGSGSGTDFTLTISRLEPEDFAVYYCQQYFNPPEYTFGQGTKVEIK

SEQ ID NO: 99 - protein, 470 aa, Artificial Sequence
  BCMA-CAR2
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVRPFWGTFDYWGQGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYFNPPEYTFGQGTKVEIKRTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 100 - protein, 512 aa, Artificial Sequence
  BCMA-CAR3
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVRPFWGTFDYWGQGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYFNPPEYTFGQGTKVEIKRTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 101 - DNA, 732 bp, Artificial Sequence
  bases of 2B1-SCFV
gaggtgcaattgctggagtctgggggaggcttggtacagcctggggggtccctgagactctcctgtgcagcctccggattcacctttagcagttatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcagatgaacagcctgagagccgaggacacggccgtatattactgtgcgaaatcttctactcgttggacttcttctactttcgactactggggccaaggaaccctggtcaccgtctcgagtggtggaggcggttcaggcggaggtggttctggcggtggcggatcggacatccagatgacccagtctccttccaccctgtctgcatctgtaggagaccgtgtcaccatcacttgccgtgccagtcagagtattagtagctggttggcctggtatcagcagaaaccagggaaagcccctaagctcctgatctatgatgcctccagtttggaaagtggggtcccatcacgtttcagcggcagtggatccgggacagaattcactctcaccatcagcagcttgcagcctgatgattttgcaacttattactgccaacagtactacttctacccatacacgtttggccagggcaccaaagtcgagatcaagcgt

SEQ ID NO: 102 - DNA, 732 bp, Artificial Sequence
  bases of 2F1-SCFV
gaggtgcaattgctggagtctgggggaggcttggtacagcctggggggtccatgagactctcctgtgcagcctccggattcacctttagcagttatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcagatgaacagcctgagagccgaggacacggccgtatattactgtgcgaaatacgaaggtcgttacgttctgggtgcaatggactactggggccaaggaaccctggtcaccgtctcgagtggtggaggcggttcaggcggaggtggttctggcggtggcggatcggacatccagatgacccagtctccttccaccctgtctgcatctgtaggagaccgtgtcaccatcacttgccgtgccagtcagagtattagtagctggttggcctggtatcagcagaaaccagggaaagcccctaagctcctgatctatgatgcctccagtttggaaagtggggtcccatcacgtttcagcggcagtggatccgggacagaattcactctcaccatcagcagcttgcagcctgatgattttgcaacttattactgccaacagtacttctcttacccactgacgtttggccagggcaccaaagtcgagatcaagcgt

(19) United States
(12) Patent Application Publication
(10) Pub. No.: US 2024/0131065 A1
(43) Pub. Date: Apr. 25, 2024

(54) CD94 ENGINEERED CELL AND COMPOSITION THEREOF

(71) Applicant: CRAGE MEDICAL CO., LIMITED, HONG KONG (CN)

(72) Inventors: Zonghai LI, Shanghai (CN); Zhimin SHI, Shanghai (CN)

(73) Assignee: CRAGE MEDICAL CO., LIMITED, HONG KONG (CN)

(21) Appl. No.: 18/278,495

(22) PCT Filed: Feb. 25, 2022
(86) PCT No.: PCT/CN2022/078005

§ 371 Date: Aug. 22, 2023

(30) Foreign Application Priority Data
Feb. 25, 2021 (CN) 202110211879.4

(51) Int. Cl. A61K 35/17; A61K 39/00; A61P 35/00; C07K 16/28; C12N 9/22

(52) U.S. Cl. CPC A61K 35/17; A61K 39/4631; A61P 35/00; C07K 16/2851; C12N 9/22; A61K 2239/13; A61K 2239/21; A61K 2239/22; C07K 2317/55; C07K 2317/622; C12N 2310/20

(57) ABSTRACT

An engineered immune cell for immunotherapy, which has a relatively high persistence and/or survival rate of transplantation in a host organism, and a preparation method therefor.

### RELATED APPLICATIONS

This application claims the priority of the Chinese application with application number 202110211879.4 filed on Feb. 25, 2021.

### FIELD OF THE INVENTION

The present application relates to a cell with anti-transplant rejection function, and also relates to a method for anti-transplant immune rejection, in particular to a method for anti-NK cell immune rejection.

### BACKGROUND OF THE INVENTION

Due to the immunogenetic differences between the donor and the recipient, when an exogenous donor is transplanted, as an exogenous transplant, the donor may be recognized and attacked by immune cells in the recipient, thereby the exogenous grafts be inhibited or eliminated, resulting in a host-versus-graft response (HVGR). By knocking out the MHC molecules in the graft cells, it can effectively resist the rejection of the graft by the host T cells, but it may cause the rejection of other immune cells in the host. For example, in allogeneic cell transplantation, the deletion of MHC-I molecules of allogeneic cells will lead to the rejection of NK cells in the host and enhance the clearance of allogeneic cells (Nat Biotechnol. 2017; 35(8): 765-772. doi:10.1038/nbt.3860). Therefore, how to effectively prevent the immune rejection of host NK cells is crucial for the development of allogeneic cell transplantation therapy.

### SUMMARY OF THE INVENTION

The purpose of this application is to provide an antibody that specifically recognizes CD94. Also provided is an anti-transplant immune rejection cell and a method for anti-transplant rejection.

According to a first aspect of the present application, provided is a genetically engineered cell expressing a first protein capable of recognizing CD94.

In a specific embodiment, the first protein comprises an antibody capable of recognizing CD94, preferably, the amino acid sequence of CD94 is shown in SEQ ID NO: 1.

In a specific embodiment, the first protein comprises a chimeric antigen receptor (CAR), a chimeric T cell receptor, a T cell antigen coupler (TAC), or a combination thereof.

In a specific embodiment, the first protein is a CAR comprising:
(i) an antibody recognizing CD94, a transmembrane domain of CD8/CD28, and CD3ζ;
(ii) an antibody recognizing CD94, a transmembrane domain of CD8/CD28, an intracellular signaling domain of CD137, and CD3;
(iii) an antibody recognizing CD94, a transmembrane domain of CD8/CD28, an intracellular signaling domain of CD28, and CD3ζ; and/or
(iv) an antibody recognizing CD94, a transmembrane domain of CD8/CD28, an intracellular signaling domain of CD28, CD137 and CD3ζ.

In a specific embodiment, the gene encoding a CD94 protein is knocked out and/or low or no expression of an endogenous CD94 molecule.

In a specific embodiment, the CD94 gene of the cell is knocked out using CRISPR/Cas9 technology, the gRNA used is selected from the sequence(s) shown as SEQ ID NO:59, 60, 61, 62, 63, 64, 65, 66, 67, 68, 69, 70 and/or 71.

In a specific embodiment, the cell is selected from a T cell, an NK cell, a cytotoxic T cell, an NKT cell, a DNT cell, an NK92 cell, a macrophage, a CIK cell, and a stem cell-derived immune effector cell, or a combination thereof.

In a specific embodiment, the cell is an autologous or allogeneic T cell, a primary T cell, or a human-derived autologous T cell.

In a specific embodiment, the cells comprise,

knockout of a gene encoding a TCR protein and/or low or no expression of an endogenous TCR molecule, and/or

knockout of a gene encoding MHC protein and/or low or no expression of an endogenous MHC.

In a specific embodiment, an endogenous MHC molecule B2M and an endogenous TCR are knocked out using CRISPR/Cas9 technology.

In a specific embodiment, the gRNA used for knocking out B2M comprises the sequence(s) shown in SEQ ID NO:55, 56, 57 and/or 58, and the gRNA used for knocking out TCR comprises the sequence(s) shown in SEQ ID NO:47, 48, 49, 50, 51, 52, 53 and/or 54.

In a specific embodiment, the first protein also recognizes a tumor and/or a pathogen; preferably, the tumor expresses including BCMA, CD19, GPC3, CLDN18A2, EGFR, or a combination thereof.

In a specific embodiment, the cell further expresses a second protein that targets and recognizes tumor antigens and/or pathogen antigens, a chemokine, a chemokine receptor, a cytokine, siRNA for reducing PD-1 expression, a protein for blocking the binding of PD-L1 to PD-1, a safety switch, or a combination thereof.

In a specific embodiment, when the cell is co-cultured with a host NK cell, the cell is capable of killing a host NK cell, or the cell is capable of resisting killing of the cell by a host NK cell, or the cell is capable of resisting killing of the cell by an activated host NK cell.

In a specific embodiment, the cell is administered in combination with an agent that enhances its function, preferably, in combination with a chemotherapeutic agent; and/or

the cell is administered in combination with an agent that ameliorates one or more side effects associated therewith; and/or

the cell is administered in combination with a cell expressing a second protein that recognizes an antigen different from a first protein.

In a specific embodiment, the second protein comprises a CAR comprising:
(i) an antibody recognizing a tumor and/or a pathogen, CD3ζ and a transmembrane domain of CD28 or CD8;
(ii) an antibody recognizing a tumor and/or a pathogen, a transmembrane domain of CD28 or CD8, a costimulatory signaling domain of CD28, and CD3ζ;
(iii) an antibody recognizing a tumor and/or a pathogen, a transmembrane domain of CD28 or CD8, a costimulatory signaling domain of CD137, and CD3ζ; and/or
(iv) an antibody recognizing a tumor and/or a pathogen, a transmembrane domain of CD28 or CD8, a costimulatory signaling domain of CD28, a costimulatory signaling domain of CD137, and CD3ζ.

In a specific embodiment, the tumor and/or pathogen expresses CLDN18A2, BCMA, CD19, GPC3, EGFR, or a combination thereof.

In a specific embodiment, the second protein comprises the sequence shown in SEQ ID NO: 74, 79, 88, 89, 90, 99 or 100.

In a specific embodiment, the cell expressing the second protein comprises:
(i) knockout of the gene encoding CD94 protein and/or low or no expression of endogenous CD94 molecule; or
(ii) knockout of the gene(s) encoding TCR and/or MHC protein(s) and/or low or no expression of the endogenous TCR and/or MHC molecule(s); or
(iii) knockout of the genes encoding CD94, TCR and MHC proteins and/or low or no expression of the endogenous CD94, TCR and MHC molecules.

In a specific embodiment, the cell expressing the second protein:
(i) knockout of endogenous CD94 molecule using CRISPR/Cas9 technology;
(ii) knockout of TCR and/or MHC molecule B2M using CRISPR/Cas9 technology; or
(iii) knockout of CD94, TCR and MHC molecule B2M using CRISPR/Cas9 technology.

In a specific embodiment, the cell expressing the second protein is selected from a T cell, an NK cell, a cytotoxic T cell, an NKT cell, a DNT cell, an NK92 cell, a macrophage, a CIK cell, and a stem cell-derived immune effector cell, or a combination thereof.

In a specific embodiment, the cell is an autologous or allogeneic T cell, a primary T cell, or a human-derived autologous T cell.

According to a second aspect of the present application, provided is a method for increasing the persistence and/or transplantation survival rate of a first immune cell in the presence of a host second immune cell, comprising:
a) providing the first immune cell;
b) optionally, modifying the first immune cell by reducing or inhibiting the expression, activity and/or signal transduction of at least one endogenous gene encoding a polypeptide involved in response to autologous and non autologous antigen recognition;
c) modifying the first immune cell by a polynucleotide encoding a first protein targeting CD94.

In a specific embodiment, the polypeptide in step b) is selected from MHC, TCR, and/or CD94.

In a specific embodiment, step b) comprises:
(i) knockout of CD94 molecule using CRISPR/Cas9 technology;
(ii) knockout of TCR and/or MHC molecule B2M using CRISPR/Cas9 technology; or
(iii) knockout of CD94, TCR and MHC molecule B2M using CRISPR/Cas9 technology.

In a specific embodiment, step b) comprises:
(i) the gRNA used to knockout CD94 molecule is selected from the sequence(s) shown in SEQ ID NO(s): 59, 60, 61, 62, 63, 64, 65, 66, 67, 68, 69, 70 and/or 71;
(ii) the gRNA used to knockout TCR comprises the sequence(s) shown in SEQ ID NO(s): 47, 48, 49, 50, 51, 52, 53 and/or 54; and/or
(iii) the gRNA used to knockout MHC molecule B2M comprises the sequence(s) shown in SEQ ID NO(s): 55, 56, 57 and/or 58.

In a specific embodiment, the first protein comprises a chimeric antigen receptor (CAR), a chimeric T cell receptor, a T cell antigen coupler (TAC), or a combination thereof.

In a specific embodiment, the first protein comprises:
(i) an antibody recognizing CD94, optionally an antibody recognizing a tumor and/or a pathogen, a transmembrane domain of CD28 or CD8, a costimulatory signaling domain of CD28, and CD3ζ; and/or
(ii) an antibody recognizing CD94, optionally an antibody recognizing a tumor and/or a pathogen, a transmembrane domain of CD28 or CD8, a costimulatory signaling domain of CD137, and CD3ζ; and/or
(iii) an antibody recognizing CD94, optionally an antibody recognizing a tumor and/or a pathogen, a transmembrane domain of CD28 or CD8, a costimulatory signaling domain of CD28, a costimulatory signaling domain of CD137, and CD3ζ;
(iv) an antibody recognizing CD94, optionally an antibody recognizing a tumor and/or a pathogen, a transmembrane domain of CD28 or CD8, and CD3ζ.

In a specific embodiment, it also comprises step d): modifying the first immune cell by a non-endogenous polynucleotide encoding a second protein targeting a tumor antigen and/or a pathogen antigen and/or a viral antigen, a chemokine, a chemokine receptor, a cytokine, siRNA for reducing PD-1 expression, a protein for blocking the binding of PD-L1 to PD-1, or a safety switch.

In a specific embodiment, the cell is selected from a T cell, an NK cell, a cytotoxic T cell, an NKT cell, a macrophage, a CIK cell, and a stem cell-derived immune effector cell, or a combination thereof.

In a specific embodiment, the first immune cell is an autologous or an allogeneic T cell, a primary T cell or a human-derived autologous T cell.

According to a third aspect of the present application, provided is an engineered cell prepared by the above-mentioned method of the present application.

According to a fourth aspect of the present application, provided is a polynucleotide encoding a nucleic acid molecule for constructing the cell of the present application or encoding a nucleic acid molecule for administrating the method of the present application.

According to a fifth aspect of the present application, provided is a vector comprising the polynucleotide as described in the present application.

According to the sixth aspect of the present application, provided is a virus comprising the vector described in the present application.

According to a seventh aspect of the present application, provided is a composition comprising an effective amount of the cell as described in the present application, the polynucleotide as described in the present application, the vector as described in the present application, and the virus as described in the present application.

According to an eighth aspect of the present application, provided is a method for treating an inflammatory disorder, viral infection and/or a tumor, comprising administering the cell as described in the present application or the composition as described in the present application to a subject in need thereof.

According to the ninth aspect of the present application, provided is an antibody recognizing CD94, wherein the antibody is selected from the group consisting of:
(1) an antibody, comprising a heavy chain variable region which comprises HCDR1 shown in SEQ ID NO: 11, and/or comprises HCDR2 shown in SEQ ID NO: 12, and/or comprises HCDR3 shown in ID NO: 13 or 17;
(2) an antibody, comprising a light chain variable region which comprises LCDR1 shown in SEQ ID NO: 14, and/or comprises LCDR2 shown in SEQ ID NO: 15, and/or comprises LCDR3 shown in SEQ ID NO: 16 or 18;
(3) an antibody, comprising a heavy chain variable region of the antibody described in (1) and a light chain variable region of the antibody described in (2);
(4) an antibody, a variant of the antibody of any one of (1)-(3), having the same or similar activity as the antibody of any one of (1)-(3).

In a specific embodiment, the antibody is selected from:
(1) an antibody, comprising HCDR1 shown in SEQ ID NO: 11, HCDR2 shown in SEQ ID NO: 12 and HCDR3 shown in SEQ ID NO: 13; LCDR1 shown in SEQ ID NO: 14, LCDR2 shown in SEQ ID NO: 15 and LCDR3 shown in SEQ ID NO: 16; or
(2) an antibody, comprising HCDR1 shown in SEQ ID NO: 11, HCDR2 shown in SEQ ID NO: 12 and HCDR3 shown in SEQ ID NO: 17; LCDR1 shown in SEQ ID NO: 14, LCDR2 shown in SEQ ID NO: 15 and LCDR3 shown in SEQ ID NO: 18;
(3) an antibody, a variant of the antibody of any one of (1)-(2), having the same or similar activity as the antibody of any one of (1)-(2).

In a specific embodiment, the antibody is selected from:
(1) an antibody, comprising a heavy chain variable region which comprises the amino acid sequence shown in any one of SEQ ID NO: 19 or 24 or a variant thereof;
(2) an antibody, comprising a light chain variable region which comprises the amino acid sequence shown in any one of SEQ ID NO: 21 or 26 or a variant thereof;
(3) an antibody, comprising the heavy chain variable region of the antibody described in (1) and the light chain variable region of the antibody described in (2).

In a specific embodiment, the antibody is selected from:
(1) an antibody, the heavy chain variable region of the antibody has the amino acid sequence shown in SEQ ID NO: 19, and the light chain variable region of the antibody has the amino acid sequence shown in SEQ ID NO: 21;
(2) an antibody, the heavy chain variable region of the antibody has the amino acid sequence shown in SEQ ID NO: 24, and the light chain variable region of the antibody has the amino acid sequence shown in SEQ ID NO: 26;
(3) an antibody, a variant of the antibody of any one of (1)-(2), having the same or similar activity as the antibody of any one of (1)-(2).

In a specific embodiment, the antibody is a whole antibody, scFv, a single domain antibody, a Fab fragment, a Fab′ fragment, a Fv fragment, a F(ab′)2 fragment, a Fd fragment, a dAb fragment or a multifunctional antibody.

According to the tenth aspect of the present application, provided is a nucleic acid encoding the antibody of the present application.

According to the eleventh aspect of the present application, provided is an expression vector comprising a nucleic acid encoding the antibody of the present application.

According to the twelfth aspect of the present application, provided is a virus comprising the expression vector of the eleventh aspect of the present application.

According to the thirteenth aspect of the present application, provided is a host cell comprising the expression vector of the eleventh aspect of the present application or the nucleic acid of the tenth aspect is integrated into the genome.

In a specific embodiment, in the cell described in the present application, the first protein comprises the antibody described in the ninth aspect of the present application; or comprises the sequence shown in SEQ ID NO: 43, 44, 45 or 46.

In a specific embodiment, the method described in this application is characterized in that the first protein comprises:
(i) the antibody described in the ninth aspect of the present application; or
(ii) the sequence shown in SEQ ID NO: 43, 44, 45 or 46.

This application also relates to:
1. An antibody binding unit that specifically recognizes CD94, having a light chain variable region and a heavy chain variable region, and,
CDR1 of its light chain variable region has an amino acid sequence shown in SEQ ID NO: 14;
CDR2 of its light chain variable region has an amino acid sequence shown in SEQ ID NO: 15;
CDR3 of its light chain variable region has an amino acid sequence shown in SEQ ID NO:16 or SEQ ID NO:18;
CDR1 of its heavy chain variable region has the amino acid sequence shown in SEQ ID NO: 11;
CDR2 of its heavy chain variable region has an amino acid sequence shown in SEQ ID NO: 12;
CDR3 of its heavy chain variable region has an amino acid sequence shown in SEQ ID NO:13, or SEQ ID NO:17.
2. The antibody binding unit of item 1, wherein, it is selected from:
an antibody binding unit (a), its light chain variable region has CDR1 shown in SEQ ID NO:14, CDR2 shown in SEQ ID NO:15, CDR3 shown in SEQ ID NO:16; and/or its heavy chain variable region has CDR1 shown in SEQ ID NO:11, CDR2 shown in SEQ ID NO:12, and CDR3 shown in SEQ ID NO:13;
an antibody binding unit (b), its light chain variable region has CDR1 shown in SEQ ID NO:14, CDR2 shown in SEQ ID NO:15, CDR3 shown in SEQ ID NO:18; and/or its heavy chain variable region has CDR1 shown in SEQ ID NO:11, CDR2 shown in SEQ ID NO:12, and CDR3 shown in SEQ ID NO:17;
an antibody binding unit (c), recognizing the same epitope as the epitope recognized by the antibody binding unit of any one of (a)-(b).
3. The antibody binding unit of item 2, wherein the amino acid sequence of the heavy chain variable region of the antibody binding unit is shown in SEQ ID NO: 19 or 24; or the amino acid sequence of the light chain variable region of the antibody binding unit is shown in SEQ ID NO:21 or 26.
4. The antigen binding unit of any one of items 1-3, wherein the antigen binding unit:
(a) having a light chain variable region shown in SEQ ID NO:21 and a heavy chain variable region shown in SEQ ID NO:19; or
(b) having a light chain variable region shown in SEQ ID NO:26 and a heavy chain variable region shown in SEQ ID NO:24.
5. The antigen binding unit of any one of items 1 to 4, wherein the antigen binding unit is scFv, Fv, Fab or (Fab)2.
6. A nucleic acid, encoding the antigen binding unit of any one of items 1-5.
7. An expression vector, comprising the nucleic acid of item 6.
8. A virus, comprising the expression vector of item 7.
9. A host cell, comprising the expression vector of item 7 or the nucleic acid of item 6 is integrated into its genome.
10. A genetically engineered cell, wherein the cell expresses a first protein capable of recognizing CD94;
preferably, the amino acid sequence of CD94 is shown in SEQ ID NO: 1;
preferably, the first protein comprises an antigen binding unit capable of recognizing CD94;
preferably, the first protein comprises: an antibody binding unit (a), and its light chain variable region has CDR1 shown in SEQ ID NO: 14, CDR2 shown in SEQ ID NO: 15, and CDR2 shown in SEQ ID NO: 16; and/or its heavy chain variable region has CDR1 shown in SEQ ID NO:11, CDR2 shown in SEQ ID NO:12, and CDR3 shown in SEQ ID NO:13; an antibody binding unit (b), its light chain variable region has CDR1 shown in SEQ ID NO:14, CDR2 shown in SEQ ID NO:15, and CDR3 shown in SEQ ID NO:18; and/or its heavy chain variable region has CDR1 shown in SEQ ID NO:11, CDR2 shown in SEQ ID NO: 12, and CDR3 shown in SEQ ID NO: 17; or an antibody binding unit (c), recognizing the same epitope as the epitope recognized by the antibody binding unit of any one of (a)-(b);
preferably, the first protein comprises the heavy chain variable region shown in SEQ ID NO: 19 or 24 and/or the light chain variable region shown in SEQ ID NO: 21 or 26;
preferably, the first protein comprises an antigen binding unit recognizing CD94, an antibody recognizing a tumor antigen or a pathogen antigen, a transmembrane domain, and an intracellular domain;
preferably, the antigen binding unit recognizing CD94 and the antibody recognizing a tumor antigen or a pathogen antigen are linked through a linker peptide.
11. The cell of item 10, wherein the cell is an immune effector cell or an engineered cell with immune effector cell function.
12. The cell of item 10 or 11, wherein the cell is selected from the group consisting of a T cell, an NK cell, an NKT cell, a macrophage, a CIK cell, and a stem cell-derived immune effector cell;
preferably, the cell is a human-derived T cell;
preferably, the cell is a human primary T cell;
more preferably, the cell is an allogeneic T cell.
13. The cell of any one of items 10-12, wherein the first protein is further linked with a cell activation signal,
preferably, the first protein comprises the TCR intracellular domain from the stimulatory domain of the intracellular signaling domain of CD3ε, CD3γ, CD3δ, TCRα or TCRβ.
14. The cell of any one of items 10-13, wherein expression, activity and/or signal transduction of the MHC and/or endogenous TCR in the cell is reduced;
preferably, the MHC is an MHC class I molecule;
more preferably, the MHC class I molecule is HLA; more preferably, the HLA is HLA-I;
more preferably, the HLA-I is selected from one or two or more of HLA-A, HLA-B, HLA-C, and B2M;
most preferably, the HLA-I comprises HLA-A and/or B2M; preferably, the endogenous TCR comprises one or two of the α and β chains of TCR;
preferably, being reduced or inhibited is performed by using a TAL nuclease, a meganuclease, a zinc finger nuclease, Cas9 and Argonaute;
preferably, the engineered T cell comprises an inhibitory nucleic acid molecule or gRNA targeting a gene encoding MHC;
preferably, the inhibitory nucleic acid molecule comprises a sequence complementary to the gene encoding MHC and/or the endogenous TCR;
preferably, the inhibitory nucleic acid comprises an RNA interfering agent;
preferably, the inhibitory nucleic acid comprises siRNA, shRNA or miRNA;
preferably, the gRNA comprises the sequence(s) shown in SEQ ID NO: 47 and/or SEQ ID NO: 55;
preferably, the reduction of the expression, activity and/or signal transduction of MHC and/or endogenous TCR is permanent, transient or inducible;
preferably, compared to the expression, activity and/or signal transduction of MHC and/or endogenous TCR in non-engineered T cell, the expression, activity and/or signal transduction reduction of MHC and/or endogenous TCR in the engineered cell is more than or greater than about 50%, 60%, 70%, 80%, 90%, 95% or 100%;
preferably, the expression of MHC and/or endogenous TCR expressed in the engineered cell is undetectable using immunoblotting assay and/or flow cytometry.
15. The cell of any one of items 10-14, wherein the first protein is selected from a chimeric antigen receptor (CAR), a chimeric T cell receptor, a T cell antigen coupler (TAC), a T Cell Fusion Protein (TFP), or a combination thereof;
preferably, the first protein comprises an extracellular domain, a transmembrane domain and an intracellular signaling domain.
16. The cell of any one of items 10-15, wherein the first protein comprises:
(i) an antigen binding unit specifically recognizing CD94, a transmembrane domain of CD28 or CD8, a costimulatory signaling domain of CD28, and CD3ζ; and/or
(ii) an antigen binding unit specifically recognizing CD94, a transmembrane domain of CD28 or CD8, a costimulatory signaling domain of CD137, and CD3ζ; and/or
(iii) an antigen binding unit specifically recognizing CD94, a transmembrane domain of CD28 or CD8, a costimulatory signaling domain of CD28, a costimulatory signaling domain of CD137, and CD3ζ;
(iv) an antigen binding unit specifically recognizing CD94, a transmembrane domain of CD28 or CD8, and CD3ζ;
preferably, the first protein comprises: an antibody binding unit (a), and its light chain variable region has CDR1 shown in SEQ ID NO: 14, CDR2 shown in SEQ ID NO: 15, and CDR3 shown in SEQ ID NO: 16; and/or its heavy chain variable region has CDR1 shown in SEQ ID NO:11, CDR2 shown in SEQ ID NO:12, and CDR3 shown in SEQ ID NO:13; an antibody binding unit (b), its light chain variable region has CDR1 shown in SEQ ID NO:14, CDR2 shown in SEQ ID NO:15, and CDR3 shown in SEQ ID NO:18; and/or its heavy chain variable region has CDR1 shown in SEQ ID NO:11, CDR2 shown in SEQ ID NO: 12, and CDR3 shown in SEQ ID NO: 17; or an antibody binding unit (c), recognizing the same epitope as the epitope recognized by the antibody binding unit of any one of (a)-(b);
preferably, the antigen binding unit specifically recognizing CD94 comprises the heavy chain variable region shown in SEQ ID NO: 19 or 24 and/or the light chain variable region shown in SEQ ID NO: 21 or 26;
preferably, the amino acid sequence of the first protein has at least 80%, preferably 90%, and more preferably 95% identity with SEQ ID NO: 23 or 28.
17. The cell of any one of items 10-16, wherein the cell further expresses a second protein targeting a tumor antigen and/or a pathogen antigen and/or a virus antigen, a chemokine, a chemokine receptor, a cytokine, siRNA for reducing PD-1 expression, a protein for blocking the binding of PD-L1 to PD-1, a safety switch, or a combination thereof;
preferably, the second protein comprises: a chimeric antigen receptor (CAR), a modified T cell (antigen) receptor (TCR), a T cell fusion protein (TFP), a T cell antigen coupler (TAC), aTCR-T, or a combination thereof;
preferably, the second protein is capable of specifically recognizing Claudin18.2, GPC3, BCMA or CD19.
18. The cell of any one of items 10-17, wherein the cell further expresses a ligand or antibody fragment of an NK cell inhibitory receptor;
preferably, the cell also expresses an NKG2A binding molecule;
preferably, the NKG2A binding molecule is a cell membrane binding protein or a secreted protein;
preferably, the NKG2A binding molecule comprises an extracellular domain, a transmembrane domain; or comprises an extracellular domain, a transmembrane domain and an intracellular domain;
preferably, the NKG2A binding molecule is an NKG2A antibody or antibody fragment bound to the cell membrane.
19. The cell of any one of items 10-18, wherein expression, activity and/or signal transduction of the endogenous CD94 in the cell is reduced or inhibited;
preferably, the reduction or inhibition is performed by using a TAL nuclease, a meganuclease, a zinc finger nuclease, Cas9 and Argonaute;
preferably, the immune cell comprises an inhibitory nucleic acid molecule or gRNA targeting a gene encoding CD94;
preferably, the inhibitory nucleic acid molecule comprises a sequence complementary to the gene encoding CD94;
preferably, the inhibitory nucleic acid comprises an RNA interfering agent;
preferably, the inhibitory nucleic acid comprises siRNA, shRNA or miRNA;
preferably, the reduction of expression, activity, and/or signal transduction of CD94 is permanent, transient, or inducible;
preferably, compared to the expression, activity and/or signal transduction of CD94 in non-engineered cell, the expression, activity and/or signal transduction reduction of CD94 in the engineered cell is more than or greater than about 50%, 60%, 70%, 80%, 90%, 95% or 100%;
preferably, the expression of CD94 expressed in the cell is undetectable using immunoblotting assay and/or flow cytometry.
20. The cell of any one of items 10-19, wherein, when the cell is co-cultured with a host NK cell, the cell is capable of killing a host NK cell.
21. The cell of any one of items 10-20, wherein when the cell is co-cultured with a host NK cell, the cell is capable of resisting killing of the cell by a host NK cell;
preferably, the cell is capable of resisting killing of the cell by a cytokine-activated NK cell in host NK cells;
preferably, the cell is capable of resisting killing of the cell by a host NK cell expressing NKG2A;
preferably, the cell can significantly resist killing of the cell by a host NK cell with low expression of NKG2A.
22. The cell of any one of items 10-21, wherein the cell is administered in combination with an agent that enhances its function, preferably in combination with a chemotherapeutic agent;
and/or the cell is administered in combination with an agent that ameliorates one or more side effects associated therewith.
23. A method for increasing the persistence and/or transplantation survival rate of an allogeneic immune cell in the presence of a host immune cell, comprising:
a) providing an allogeneic cell,
b) modifying the cell by reducing or inhibiting the expression, activity and/or signal transduction of at least one endogenous gene encoding a polypeptide involved in the response to self and non-self antigen recognition,
c) modifying the cell by a polynucleotide encoding a first protein targeting CD94;
preferably, the amino acid sequence of CD94 is shown in SEQ ID NO: 1;
preferably, the first protein comprises an antigen binding unit capable of recognizing CD94;
preferably, the first protein comprises the heavy chain variable region shown in SEQ ID NO: 19 or 24 and/or the light chain variable region shown in SEQ ID NO: 21 or 26;
preferably, the first protein comprises an antigen binding unit recognizing CD94, an antibody recognizing a tumor antigen or a pathogen antigen, a transmembrane domain, and an intracellular domain;
preferably, the antigen binding unit recognizing CD94 and the antibody recognizing a tumor antigen or a pathogen antigen are linked through a linker peptide.
24. The method of item 23, wherein the polypeptide in step b) is selected from MHC and/or endogenous TCR, and the MHC is an MHC class I molecule; more preferably, the MHC class I molecule is HLA; more preferably, the HLA is HLA-I; more preferably, the HLA-I is selected from one or two or more of HLA-A, HLA-B, HLA-C, and B2M; preferably, the endogenous TCR comprises one or two of the a and (3 chains of TCR; more preferably, the HLA-I comprises HLA-A and/or B2M;
preferably, step b) modifies the cell by reducing or inhibiting expression, activity and/or signal transduction of B2M and TCR;
preferably, the reduction or inhibition is performed by using a TAL nuclease, a meganuclease, a zinc finger nuclease, Cas9 and Argonaute;
preferably, the cell comprises an inhibitory nucleic acid molecule or gRNA targeting a gene encoding WIC;
preferably, the inhibitory nucleic acid molecule comprises a sequence complementary to the gene encoding the MHC;
preferably, the inhibitory nucleic acid comprises an RNA interfering agent;
preferably, the inhibitory nucleic acid comprises siRNA, shRNA or miRNA;
preferably, the gRNA sequence comprises the sequence(s) shown in SEQ ID NO: 47 and/or 55;
preferably, the reduction of the expression, activity and/or signal transduction of MHC and/or endogenous TCR is permanent, transient or inducible;
preferably, compared to the expression, activity and/or signal transduction of MHC and/or endogenous TCR in non-engineered T cell, the expression, activity and/or signal transduction reduction of MHC and/or endogenous TCR in the engineered cell is more than or greater than about 50%, 60%, 70%, 80%, 90%, 95% or 100%;
preferably, the expression of MHC expressed in the cell is undetectable using immunoblotting assay and/or flow cytometry.
25. The method of item 23 or 24, wherein the first protein is selected from a chimeric antigen receptor (CAR), a chimeric T cell receptor, a T cell antigen coupler (TAC), a T Cell Fusion Protein (TFP), or a combination thereof;
preferably, the first protein comprises an extracellular domain, a transmembrane domain and an intracellular signaling domain;
preferably, the cells transmit signals through the intracellular signaling domain to mediate the inhibition or killing of immune effector cells of the host;
preferably, the first protein comprises:
(i) an antigen binding unit specifically recognizing CD94, a transmembrane domain of CD28 or CD8, a costimulatory signaling domain of CD28, and CD3ζ; and/or
(ii) an antigen binding unit specifically recognizing CD94, a transmembrane domain of CD28 or CD8, a costimulatory signaling domain of CD137, and CD3ζ; and/or
(iii) an antigen binding unit specifically recognizing CD94, a transmembrane domain of CD28 or CD8, a costimulatory signaling domain of CD28, a costimulatory signaling domain of CD137, and CD3ζ;
(iv) an antigen binding unit specifically recognizing CD94, a transmembrane domain of CD28 or CD8, and CD3ζ;
preferably, the first protein comprises: an antibody binding unit (a), and its light chain variable region has CDR1 shown in SEQ ID NO: 14, CDR2 shown in SEQ ID NO: 15, and CDR3 shown in SEQ ID NO: 16; and/or its heavy chain variable region has CDR1 shown in SEQ ID NO:11, CDR2 shown in SEQ ID NO:12, and CDR3 shown in SEQ ID NO:13; an antibody binding unit (b), its light chain variable region has CDR1 shown in SEQ ID NO:14, CDR2 shown in SEQ ID NO:15, and CDR3 shown in SEQ ID NO:18; and/or its heavy chain variable region has CDR1 shown in SEQ ID NO:11, CDR2 shown in SEQ ID NO: 12, and CDR3 shown in SEQ ID NO: 17; or an antibody binding unit (c), recognizing the same epitope as the epitope recognized by the antibody binding unit of any one of (a)-(b);
preferably, the antigen binding unit specifically recognizing CD94 comprises the heavy chain variable region shown in SEQ ID NO: 19 or 24 and/or the light chain variable region shown in SEQ ID NO: 21 or 26;
preferably, the amino acid sequence of the first protein has at least 80%, preferably 90%, and more preferably 95% identity with SEQ ID NO: 23 or 28.
26. The method of any one of items 23-25, further comprising step d): modifying the cell by non-endogenous polynucleotides encoding a second protein targeting a tumor antigen and/or a pathogen antigen and/or a virus antigen, a chemokine, a chemokine receptor, a cytokine, siRNA for reducing PD-1 expression, a protein for blocking the binding of PD-L1 to PD-1, or a safety switch etc.;
preferably, the second protein comprises: a chimeric antigen receptor (CAR), a modified T cell (antigen) receptor (TCR), a T cell fusion protein (TFP), a T cell antigen coupler (TAC), aTCR-T, or a combination thereof;
preferably, the second protein is capable of specifically recognizing Claudin18.2, GPC3, BCMA or CD19.
27. The method of any one of items 23-26, further comprising step e): modifying the cell by a non-endogenous polynucleotide encoding a ligand or antibody fragment of an NK cell inhibitory receptor;
preferably, further comprising step e): modifying the cell by a non-endogenous polynucleotide encoding an NKG2A binding molecule of the immune cell;
preferably, the NKG2A binding molecule is a cell membrane binding protein or a secreted protein;
Preferably, the NKG2A binding molecule comprises only an extracellular domain, a transmembrane domain; or comprises an extracellular domain, a transmembrane domain and an intracellular domain;
preferably, the NKG2A binding molecule is an NKG2An antibody or antibody fragment bound to the cell membrane.
28. The method of any one of items 23-27, further comprising step 0: modifying the cell by reducing or inhibiting the expression, activity and/or signal transduction of the gene encoding CD94;
preferably, the reduction or inhibition is performed by using a TAL nuclease, a meganuclease, a zinc finger nuclease, Cas9 and Argonaute;
preferably, the cell comprises an inhibitory nucleic acid molecule or gRNA targeting the gene encoding CD94;
preferably, the inhibitory nucleic acid molecule comprises a sequence complementary to the gene encoding CD94;
preferably, the inhibitory nucleic acid comprises an RNA interfering agent;
preferably, the inhibitory nucleic acid comprises siRNA, shRNA or miRNA;
preferably, the reduction of expression, activity, and/or signal transduction of CD94 is permanent, transient or inducible;
preferably, compared to the expression, activity and/or signal transduction of CD94 in non-engineered cell, the reduction of expression, activity and/or signal transduction of CD94 in the engineered cell is more than or greater than about 50%, 60%, 70%, 80%, 90%, 95% or 100%;
preferably, the expression of CD94 expressed in the immune cell is undetectable using immunoblotting assay and/or flow cytometry.
29. The method of any one of items 23-28, wherein the cells are selected from the group consisting of a T cell, an NK cell, an NKT cell, a macrophage, a CIK cell, and a stem cell-derived immune effector cell;
preferably, the cell is a human-derived T cell;
preferably, the cell is a human primary T cell;
more preferably, the cell is an allogeneic T cell.
30. The method of any one of items 23 to 29, wherein when the cell prepared by the method is co-cultured with a host NK cell, the cell is capable of killing a host NK cell.
31. The method according to any one of items 23-30, wherein when the cell prepared by the method is co-cultured with host NK cells, the cell is capable of resisting killing of the cell by host NK cells;
preferably, the cell is capable of resisting killing of the cell by cytokine-activated NK cells in host NK cells;
preferably, the cell is capable of resisting killing of the cell by a host NK cell expressing NKG2A;
preferably, the cell is capable of significantly resisting killing of the cell by a host NK cell with low expression of NKG2A.
32. The method of any one of items 23-31, wherein the cell is administered in combination with an agent that enhances its function, preferably, in combination with a chemotherapeutic agent;
or the cell prepared by the method is administered in combination with an agent that ameliorates one or more side effects associated therewith.
33. An engineered cell, preparing by the method of any one of items 23-32.
34. A bispecific antibody construct, comprising a first binding domain of human or cynomolgus CD94 bound to the surface of a target cell and a second binding domain of human CD3 bound to the surface of a T cell.
35. The antibody construct of item 34, wherein the second binding domain binds to human CD3ε and CD3ε of common marmoset, cotton-top tamarin or Saimiri sciureus;
preferably, the antibody construct is selected from the following formats: (scFv)2, a scFv-single domain mAb, a diabody and an oligomer of these formats.
36. A polynucleotide, encoding the antibody construct of item 34 or 35, or encoding the construct of the cell of any one of items 10-22, or encoding a construct needed when applying the method of any one of items 23-32.
37. A vector, comprising a polynucleotide as defined in item 36.
38. A virus infected with the vector described in item 37.
39. A composition, comprising an effective amount of the engineered cell of any one of items 10-22 or 33;
preferably, further comprising a pharmaceutically acceptable carrier;
preferably, the carrier is saline solution, dextrose solution or 5% human serum albumin. preferably, further comprising a cryoprotectant.
40. A kit, comprising the engineered cell of any one of items 10-22 or 33, or the composition of item 39, and an additional agent for treating a disease.
41. A method for treating a disease, comprising administering the engineered cell of any one of items 10-22 or 33 or the composition of item 39 or the kit of item 40 to a subject in need thereof
preferably, the engineered cell is produced by the method of any one of items 23-32 prior to administration of the engineered cell;
preferably, further comprising administering an additional agent;
preferably, the disease is selected from an inflammatory disorder, an infection and a tumor;
preferably, the subject is a human;
preferably, wherein the engineered cells are autologous or allogeneic T cells to the subject.

It should be understood that within the scope of the present application, the above-mentioned technical features of the present application and the technical features specifically described in the following (e.g., the Examples) can be combined with each other to constitute new or preferred technical solutions. Due to space limitations, it is not repeated here.

### BRIEF DESCRIPTION OF THE DRAWINGS

FIG. 1A shows the electrophoresis of the heterodimeric recombinant protein of CD94 and NKG2A; FIG. 1B shows the electrophoresis of the NKG2A monomeric Fc fusion protein and the CD94 monomeric Fc fusion protein.

FIG. 2 shows ELISA results of CD94-specific antibody.

FIG. 3 shows the expression of CD94 on the surface of NK cells.

FIG. 4 shows the percentage of CD94+NK cells in activated NK cells;

FIG. 5 shows the expression ratio of CD94 in T cells, B cells and monocytes in the peripheral blood of 7 donors;

FIG. 6 shows the map of CAR vectors of 2F1-28Z, 2F1-BBZ, 2B1-28Z, 2B1-BBZ;

FIG. 7 shows the resistance function of U-2F1-28Z CAR-T cells, U-2F1-BBZ CAR-T cells, and U-2B1-28Z CAR-T cells to resting NK cells;

FIG. 8 shows the resistance function of U-2F1-28Z CAR-T cells, U-2F1-BBZ CAR-T cells, and U-2B1-28Z CAR-T cells to activated NK cells;

FIG. 9 shows the proportion of T cells and NK cells in co culture of U-2F1-28Z CAR-T cells, U-2F1-BBZ CAR-T cells, U-2B1-28Z CAR-T cells, U-2B1-BBZ CAR-T cells with PBMC cells for 0 h, 24 h and 48 h;

FIG. 10 shows the knockout efficiency of 13 CD94 gRNAs on the CD94 gene;

FIG. 11 shows a map of the CLDN18A2-CAR vector;

FIG. 12A shows the CAR positive rate of CLDN18A2 CAR-T cells and CLDN18A2 CAR-T-CD94 KO cells; FIG. 12B shows that knockout of CD94 gene does not affect the in vitro activity of CLDN18A2 UCAR-T cells;

FIG. 13 shows a map of the BCMA-CAR vector;

FIG. 14 shows the CAR positive rate of BCMA-CAR-T cells and BCMA-CAR-T-CD94 KO cells;

FIG. 15 shows that knockout of CD94 gene does not affect the therapeutic effect of BCMA-CAR-T on PRMI8226 transplanted tumors in mice;

FIG. 16 shows the detection of CD94 expression in BCMA-CAR-T cells and BCMA-CAR-T-CD94 KO cells by APC-CD3 antibody+PE-CD94 antibody+BV421-CD4 antibody;

FIG. 17 shows the CAR positive rate of BCMA-U CAR-T-CD94 KO cells and U-2F1-28Z CAR-T-CD94 KO cells;

FIG. 18 shows that in the presence of NK cells, the effect of BCMA-UCAR-T-CD94 KO cells alone or combined use of BCMA-UCAR-T-CD94 KO cells and U-2F1-28Z CAR-T-CD94 KO cells on the therapeutic effect of PRMI8226 transplanted tumors in mice;

FIG. 19 shows that in the presence of NK cells, effect of BCMA-UCAR-T-CD94 KO cell alone or combined use of BCMA-UCAR-T-CD94 KO cells and CD94 KO cells on the survival of T cells in peripheral blood after treatment of PRMI8226 transplanted tumors in mice.

### DETAIL DESCRIPTION OF THE APPLICATION

After wide and deep research, the inventor unexpectedly found that expression of a chimeric antigen receptor targeting CD94 on the surface of immune effector cells or engineered cells with immune effector cell function can resist the attack of host NK cells on allogeneic immune effector cells or engineered cells with immune effector cell function, prolong the survival time of engineered allogeneic T cells expressing chimeric antigen receptor targeting CD94 in the host, and improve anti-tumor effect. This application is completed on this basis.

### Terms

Unless specifically defined, all technical and scientific terms used herein have the same meaning as commonly understood by those skilled in the arts of gene therapy, biochemistry, genetics and molecular biology. All methods and materials similar or equivalent to those described herein can be used in the practice or testing of the present application, where suitable methods and materials are described herein. All publications, applications, and other references mentioned herein are incorporated by reference in their entirety. In case of conflict, the present specification, including definitions, will control. Furthermore, unless otherwise specified, the materials, methods, and examples are illustrative only and not intended to be limiting.

Unless otherwise indicated, the practice of this application will employ conventional techniques of cell biology, cell culture, molecular biology, transgenic biology, microbiology, recombinant DNA, and immunology, which are within the skill of the art. These techniques are fully explained in the literature. See, e.g., Current Protocols in Molecular Biology (FrederickM.AUSUBEL, 2000, Wiley and son Inc, Library of Congress, USA); Molecular Cloning: A Laboratory Manual, Third Edition, (Sambrooketal, 2001, Cold Spring Harbor, New York: Cold Spring Harbor Laboratory Press); Oligonucleotide Synthesis (M. J. Gaited., 1984); Mullis et al. U.S. Pat. No. 4,683,195; Nucleic Acid Hybridization (B. D. Harries & S. J. Higginseds. 1984); Transcription And Translation (B. D. Hames & S. J. Higginseds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), especially Vols. 154 and 155 (Wu et al. eds.) and Vol. 185, “Gene Expression Technology” (D. Goeddel, ed.); Gene Transfer Vectors For Mammalian Cells (J. H. Miller and M. P. Caloseds., 1987, Cold Spring Harbor Laboratory); Immunochemical Methods In Cell And Molecular Biology (Mayer and Walker, eds., Academic Press, London, 1987); Hand book Of Experimental Immunology, Vols. I-IV (D. M. Weir and C. C. Blackwell, eds., 1986); and Manipulating the Mouse Embryo (Cold Spring Harbor Laboratory Press, Cold Spring Harbor, N.Y., 1986).

Throughout the disclosure, various aspects of the claimed subject matter are presented in range format. It should be understood that the description in range format is merely for convenience and brevity and should not be construed as an inflexible limitation on the scope of the claimed subject matter. Accordingly, the description of a range should be considered to have specifically disclosed all possible subranges as well as individual numerical values within that range. For example, where a range of values is provided, it should be understood that every intervening value between the upper and lower limit of the range, as well as any other stated or intervening value in that range, is included within the claimed subject matter, the upper and lower limits of the range also belong to the scope of the claimed subject matter. The upper and lower limits of these smaller ranges may independently be included in the smaller ranges, and are also within the scope of the claimed subject matter, unless the upper and lower limits of the stated ranges are expressly excluded. Where the stated range comprises one or two thresholds, the claimed subject matter also comprises ranges excluding the one or two thresholds. This applies regardless of the width of the range.

The term “about” as used herein refers to the usual error range for each value readily known to those skilled in the art. Reference herein to “about” a value or parameter comprises (and describes) embodiments directed to that value or parameter itself. For example, description of “about X” comprises description of “X”. For example, “about” or “comprising” may mean within 1 or more than 1 according to the actual standard deviation in the field. Alternatively “about” or “comprising” may mean a range of up to 10% (i.e., ±10%). For example, about 5 uM can comprise any number between 4.5 uM and 5.5 uM.

Unless otherwise indicated, any concentration range, percentage range, ratio range, or integer range described herein should be understood to comprise any integer within the stated range, as well as, where appropriate, fractions thereof (e.g., one tenth and one percent of an integer).

For a better understanding of this application, the relevant terms are defined as follows:

CD94 protein, also known as NK cell antigen CD94, is mainly expressed on the surface of NK cells and partially activated T cells. The peptide of GenBank accession number GeneID:3824 (mRNA: NM 002262.5), the amino acid sequence of human CD94 is shown in SEQ ID NO:13. CD94 can form complexes with NKG2C and NKG2A, respectively. The complex of CD94 and NKG2C is an activating receptor of NK cells, and the complex of CD94 and NKG2A is an inhibitory receptor of NK cells. Both complexes can combine with HLA-E molecule polypeptide complexes of MHC-I molecules, thereby transmitting signals for activating or inhibiting NK cells. The term “CLDN18A2” refers to Claudin 18 (CLD18) molecules (Genbank accession numbers: splice variant 1 (CLD18A1): NP 057453, NM016369, and splice variant 2 (CLD18A2): NM 001002026, NP 001002026) that are intrinsic transmembrane protein with a molecular weight of about 27,9/27,72 kD. Claudin is an intrinsic membrane protein located in the tight junctions of the epithelium and endothelium. Tight junctions organize a web of interconnected chains of particles within the membrane between adjacent cells.

The term “CD19” is also referred to as B cell surface antigen B4, B cell antigen CD19, CD19 antigen or Leu-12, including but not limited to variants, isotypes and species homologues of human CD19. The complete amino acid sequence of an exemplary human CD19 has SwissPort accession number P15391. Human CD19 is referred to by Entrez Gene as Gene ID: 930 and by HGNC as HGNC: 1633.

The term “GPC3” is Glypican-3 (also known as DGSX, GTR2-2, MXR7, OCI-5, SDYS, SGB, SGBS or SGBS1), a cell surface protein that is a heparan sulfate proteoglycan family. The GPC3 gene encodes a precursor core protein of around 70-kDa that can be cleaved by furin to produce a soluble amino-terminal (N-terminal) peptide of around 40-kDa that can enter the blood and a membrane binding carboxyl-terminal (C-terminal) peptide of around 30-kDa comprising two heparan sulfate (HS) sugar chains.

The term “EGFR” refers to epidermal growth factor receptor (abbreviated as EGFR, ErbB-1 or HER1), and can also refer to homologues, orthologues, and interspecies homologues, codon-optimized forms, truncated forms, fragmented forms, mutated forms or any other known derivative forms of known EGFR sequences, such as a post-translationally modified variant. The EGFR signaling pathway plays an important role in physiological processes such as cell growth, proliferation and differentiation. In some embodiments, the epidermal growth factor EGFR is a peptide with GenBank accession No. GeneID: 1956 (mRNA: NM_005228).

The term “EGFRvIII” (EGFR mutant III, de2-7-EGFR, ΔEGFR or Δ2-7) may also refer to homologues, orthologues, interspecies homologues, codon-optimized forms, truncated forms, fragmented forms, mutated forms, or any other known derived forms of known EGFRvIII sequences, such as a post-translationally modified variant. EGFRvIII is a truncated peptide missing positions 6-273 of the EGFR peptide, and its expression has been detected in many tumors, including glioma, breast cancer, lung tumor, ovarian cancer, and prostate cancer. EGFRvIII expression is often detected in tumor cells with EGFR overexpressing or EGFR amplification.

The term “antibody” is used herein in the broadest sense and comprises a variety of antibody structures, including, but not limited to, monoclonal antibodies, polyclonal antibodies, multispecific antibodies (e.g., bispecific antibodies) and antibody fragments which can specifically bind an antigen or epitope, as long as it exhibits the desired antigen-binding activity.

An “antibody fragment” refers to a molecule other than an intact antibody that comprises the portion of the intact antibody that binds the antigen to which the intact antibody binds. Examples of antibody fragments comprise, but are not limited to (i) Fab fragments consisting of VL, VH, CL and CH1 domains, including Fab′ and Fab′-SH, (ii) Fd fragments consisting of VH and CH1 domains, (iii)) Fv fragments consisting of the VL and VH domains of a single antibody; (iv) dAb fragments consisting of a single variable region (Ward et al., 1989, Nature 341:544-546); (v) F(ab′)2 Fragments, bivalent fragments comprising 2 linked Fab fragments; (vi) antigen binding site of single-chain Fv molecule; (vii) bispecific single-chain Fv dimers (PCT/US92/09965); (viii) “dibody” or “tribody”, a multivalent or multispecific fragment constructed by gene fusion; and (ix) an scFv genetically fused to the same or a different antibody.

The “class” of an antibody refers to the type of the constant domain or constant region possessed by its heavy chain. There are mainly five classes of antibodies: IgA, IgD, IgE, IgG, and IgM, and some of these can be further divided into subclasses (allotypes), e.g., IgG1, IgG2, IgG3, IgG4, IgA1, and IgA2. The heavy chain constant domains that correspond to the different classes of immunoglobulins are called alpha, delta, epsilon, gamma, and mu, respectively.

The term “variable region or variable domain” refers to a domain of an antibody heavy or light chain that is involved in antibody antigen binding. The heavy and light chain variable domains (VH and VL, respectively) of native antibodies generally have similar structures, wherein each domain comprising four conserved FRs and three CDRs. (See, e.g., Kindt et al., Kuby Immunology, 6th ed., W.H. Freeman & Co., p. 91 (2007)). A single VH or VL domain may be sufficient to confer antigen binding specificity. In addition, antibodies that bind a particular antigen can be isolated by screening a library of complementary VL or VH domain using the VL or VH domain, respectively, from antibodies that bind to the antigen. See, e.g., Portolano et al., J. Immunol. 150:880-887 (1993); Clarkson et al., Nature 352:624-628 (1991).

The term “hypervariable region” or “complementarity determining region” or “CDR” refers to the various regions in the variable domain of an antibody where the sequence is hypervariable and/or forms a structurally defined loop (“hypervariable loop”) and/or comprises residues in contact with an antigen (“antigen contact”). Typically, an antibody comprises six CDRs: three in the VH (HCDR1, HCDR2, HCDR3) and three in the VL (LCDR1, LCDR2, LCDR3).

The terms “Fc region” or “Fc” are used to define the C-terminal region of an immunoglobulin heavy chain comprising at least a portion of the constant region. The term comprises Fc regions of native sequences and variants.

“Framework (FR)” refers to variable domain residues other than residues of hypervariable region (CDR). The FRs of variable domains generally consist of four FR domains: FR1, FR2, FR3 and FR4. Thus, in VH (or VL), the CDR and FR sequences generally appear in the following order: FR1-HCDR1(LCDR1)-FR2-HCDR2(LCDR2)-FR3-HCDR3(LCDR3)-FR4.

Unless otherwise indicated, herein, CDR residues and other residues in the variable domains (e.g., FR residues) are numbered according to Kabat etc.

The term “native antibody” refers to a naturally occurring immunoglobulin molecule with a variety of structures. For example, native IgG antibodies are heterotetrameric glycoproteins of approximately 150,000 Daltons, consisting of two identical light chains and two identical heavy chains bonded by disulfide bonds. From the N-terminus to the C-terminus, each heavy chain has variable regions (VH), also known as the variable heavy chain domain or the heavy chain variable domain, followed by three constant domains (CH1, CH2 and CH3). Similarly, from the N-terminus to the C-terminus, each light chain has variable regions (VL), also known as the variable light chain domain or light chain variable domain, followed by the light chain constant (CL) domain. The light chains of antibodies can be assigned to one of two types, called kappa (κ) and λ (λ), based on the amino acid sequence of their constant domains.

The terms “whole antibody”, “full length antibody”, “intact antibody” are used interchangeably and refer to the intact full-length antibody having a structure substantially similar to that of a native antibody or having a heavy chain comprising an Fc region as defined herein or including full-length antibody having antigen binding region.

The term “single domain antibody (sdAb)” refers to a type of antibody that lacks the light chain of the antibody and only has the heavy chain variable region. Because of its small molecular weight, it is also called Nanobody.

The term “monodomain antibody” refers to an antibody comprising all or part of the heavy chain variable domain or all or part of the light chain variable domain. In certain embodiments, the monodomain antibody is a human monodomain antibody (Domantis, Inc., Waltham, MA; see, e.g., US Application No. 6248516).

The antibodies of the present application can be isolated by screening combinatorial libraries of antibodies having one or more desired activities. For example, various methods known in the art can be used to generate phage display libraries and screen the libraries for antibodies with desired binding properties. Such methods are reviewed, for example, in Hoogenboom et al., Methods in Molecular Biology 178:1-37 (O'Brien et al., Human Press, Totowa, N J, 2001) and further described in, for example, McCafferty et al., Nature 348:552-554; Clackson et al, Nature 352: 624-628 (1991); Marks et al, J. Mol. Biol. 222: 581-597 (1992); Marks, Meth. Mol. Biol., 248: 161-175 (Lo, ed., Human Press, Totowa, N J, 2003); Sidhu et al., J. Mol. Biol. 338(2):299-310(2004); Lee et al., J. Mol. Biol. 340(5):1073-1093(2004); Fellouse, Proc. Natl. Acad. Sci. USA 101(34): 12467-12472 (2004); Lee et al., J. Immunol. Methods 284(1-2): 119-132 (2004).

In some phage display methods, VH and VL gene libraries are separately cloned by polymerase chain reaction (PCR) and randomly recombined in a phage library, which can then be screened for antigen-binding phage, as described in Winter et al., Ann. Rev. Immunol. 12:433-455 (1994). Phages typically display antibody fragments as single-chain Fv (scFv) fragments or Fab fragments. Libraries from immunized sources provide high affinity antibodies to the immunogen without need to construct hybridomas. Alternatively, native libraries can be cloned (e.g., from humans) to provide antibodies of a single source to multiple non autoantigens as well as autoantigens without any immunization, as described in Griffiths et al., EMBO J, 12:725-734 (1993). Finally, native libraries can also be prepared synthetically by cloning unrearranged V-gene segments from stem cells and using PCR primers comprising random sequences to encode hypervariable CDR3 regions and achieve rearrangement in vitro, as described in Hoogenboom, J. Mol. Biol. 227:381-388 (1992).

The term “variant” refers to a polypeptide having substantially the same amino acid sequence or one or more active polypeptide encoded by substantially the same nucleotide sequence as the sequences of the antibodies provided herein. The variants have the same or similar activities as the antibodies provided in the examples of the present application.

The terms “variant antibody” or “antibody variant” comprise antibody sequences that differ from the parent antibody sequence due to at least one amino acid modification compared to the parent. Variant antibody sequences herein preferably have at least about 80%, 81%, 82%, 83%, 84%, 85%, 86%, 87%, 88%, 89%, 90%, 91%, 92%, 93%, 94%, 95%, 96%, 97%, 98%, 99% amino acid sequence identity with the parent antibody sequence. An antibody variant may refer to the antibody itself or to a composition comprising the antibody variant. Amino acid sequence variants of an antibody can be prepared by introducing appropriate modifications to the nucleotide sequence encoding the antibody or by peptide synthesis. The term “amino acid modification” comprises amino acid substitutions, additions and/or deletions, “amino acid substitution”, “amino acid replacement” means replacing an amino acid at a specified position in the parent polypeptide sequence with another amino acid, and “amino acid insertion” means the addition of an amino acid at a specific position in the parent polypeptide sequence, and “amino acid deletion” means removal of an amino acid at a specific position in the parent polypeptide sequence. Any combination of deletions, insertions and substitutions can be made to obtain the final construct, provided that the final construct has the desired characteristics, e.g.: binding to antigen.

The term “scFv” refers to a fusion protein comprising at least one antibody fragment comprising a light chain variable region and at least one antibody fragment comprising a heavy chain variable region, wherein the light and heavy chain variable regions are contiguous (for example, via a synthetic linker, such as a short flexible polypeptide linker), and can be expressed as a single-chain polypeptide, and wherein the scFv retains the specificity of the intact antibody from which it is derived. Unless specified, as used herein, a scFv may have the VL and VH variable regions described in any order (e.g., with respect to the N-terminus and C-terminus of the polypeptide), the scFv may comprise a VL-linker-VH or VH-linker-VL.

As used herein, “antigen” refers to a substance that is recognized and specifically bound by an antibody. Antigens can comprise peptides, proteins, glycoproteins, polysaccharides, and lipids, portions thereof, and combinations thereof. Non-limiting exemplary antigens comprise tumor antigens or pathogen antigens. “Antigen” can also refer to a molecule that elicits an immune response. This immune response may involve antibody production or activation of specific immunologically-competent cells, or both. Those skilled in the art will appreciate that any macromolecule, including virtually any protein or peptide, can serve as an antigen.

The term “epitope” refers to an antigen or part of an antigen that can be recognized by an antibody, B cell, T cell or engineered cell. For example, an epitope may be a tumor epitope or a pathogen epitope recognized by an antibody; the antibody recognizes multiple epitopes within an antigen. Epitopes can also be mutated.

The term “antigenic determining site”, also known as “antigenic epitope” or “epitope” or “antigenic determinant”, comprises any determinant or region capable of being bound by an antibody. An epitope is a region of an antigen that is bound by an antibody targeting the antigen and comprises specific amino acids that are in direct contact with the antibody. Exemplarily, the antigenic epitope can be composed of a contiguous sequence of CD94 protein sequences, or it can be composed of three-dimensional structures of non-contiguous CD94 protein sequences. Exemplarily, the antigen used herein is human CD94, the amino acid sequence of which is shown in SEQ ID NO:1.

The terms “polypeptide,” “peptide,” and “protein” are used interchangeably herein to refer to polymers of amino acids of any length. The polymer may be linear, cyclic or branched, it may comprise modified amino acids, especially conservatively modified amino acids, and it may be interrupted by non-amino acids. The term also comprises modified amino acid polymers such as those that have been processed by sulfation, glycosylation, lipidation, acetylation, phosphorylation, iodination, methylation, oxidation, proteolytic processing, prenylation, racemization, selenoacylation, transfer-RNA mediated amino addition such as arginylation, ubiquitination, or any other manipulation such as conjugation to labeling components. As used herein, the term “amino acid” refers to natural and/or unnatural or synthetic amino acids, including glycine and D or L optical isomers, as well as amino acid analogs and peptidomimetics. A polypeptide or amino acid sequence “derived from” a specified protein refers to the source of the polypeptide. The term also comprises polypeptides expressed from the specified nucleic acid sequence.

The term “conservative modification” or “conservative sequence modification” as used herein means amino acid modifications that do not significantly affect or alter the desired activity or property of the peptide comprising the amino acid sequence. Such conservative modifications comprise amino acid substitutions, insertions and deletions.

The terms “anti-CD94 antibody”, “antibody binding CD94”, “CD94 antibody”, “antibody recognizing CD94” refer to an antibody capable of binding CD94 with sufficient affinity for use as a diagnostic agent and/or therapeutic agent for targeting CD94. In one embodiment, the degree of the binding of an anti-CD94 antibody to an unrelated, non-CD94 protein is less than about 10% of the degree of binding of the antibody to CD94, as determined by an enzyme-linked immunosorbent assay (ELISA). In certain embodiments, the anti-CD94 antibody binds to an epitope of CD94 that is conserved among CD94 derived from different species.

The present application uses the human CD94 monomeric Fc fusion protein antigen to select Fab from a fully human native Fab phage library. These molecules exhibit fine specificity. For example, the antibody only recognizes CD94 but not NKG2A. Unless otherwise specified in this application, CD94 herein refers to human CD94.

The antibodies or antibody fragments of the present application are based on the use of phage display to identify and select antigen-binding fragments (Fabs) whose amino acid sequences confer specificity for the antibody or antibody fragment against CD94 and form the basis of all antigen-binding proteins of the present disclosure. Thus, the Fab can be used to design a series of different “antibodies or antibody fragments”.

The application provides an antibody recognizing CD94, the antibody comprises a heavy chain variable region comprising the heavy chain CDR1 of the amino acid sequence shown in SEQ ID NO: 11, and/or comprising the heavy chain CDR2 of the amino acid sequence shown in SEQ ID NO: 12, and/or comprising the heavy chain CDR3 of the amino acid sequence shown in SEQ ID NO: 13 or 17. In another preferred embodiment, the application provides an antibody recognizing CD94, comprising: the light chain CDR1 comprising the amino acid sequence shown in SEQ ID NO: 14, and/or the light chain CDR2 comprising the amino acid sequence shown in SEQ ID NO: 15, and/or a light chain CDR3 comprising the amino acid sequence shown in SEQ ID NO: 16 or 18. In another preferred embodiment, the present application provides an antibody recognizing CD94, comprising: the heavy chain CDR1 comprising the amino acid sequence shown in SEQ ID NO: 11, and/or the heavy chain CDR2 comprising the amino acid sequence shown in SEQ ID NO: 12, and/or the heavy chain CDR3 comprising the amino acid sequence shown in SEQ ID NO: 13 or 17, and/or the light chain CDR1 comprising the amino acid sequence shown in SEQ ID NO: 14, and/or the light chain CDR2 comprising the amino acid sequence shown in SEQ ID NO: 15, and/or the light chain CDR3 comprising the amino acid sequence shown in SEQ ID NO: 16 or 18. Preferably, the antibody recognizing CD94 comprises: the heavy chain CDR1 comprising the amino acid sequence shown in SEQ ID NO: 11, the heavy chain CDR2 comprising the amino acid sequence shown in SEQ ID NO: 12, and the heavy chain CDR3 comprising the amino acid sequence shown in SEQ ID NO: 13 or 17, and/or the light chain CDR1 comprising the amino acid sequence shown in SEQ ID NO: 14, the light chain CDR2 comprising the amino acid sequence shown in SEQ ID NO: 15, and the light chain CDR3 comprising the amino acid sequence shown in SEQ ID NO: NO: 16 or 18. More preferably, the antibody recognizing CD94 comprises: the heavy chain CDR1 comprising the amino acid sequence shown in SEQ ID NO:11, the heavy chain CDR2 comprising the amino acid sequence shown in SEQ ID NO:12, and the heavy chain CDR3 comprising the amino acid sequence shown in SEQ ID NO:13 or 17, and the light chain CDR1 comprising the amino acid sequence shown in SEQ ID NO: 14, the light chain CDR2 comprising the amino acid sequence shown in SEQ ID NO: 15, and the light chain CDR3 comprising the amino acid sequence shown in SEQ ID NO: 16 or 18. More preferably, the antibody recognizing CD94 comprises: HCDR1 shown in SEQ ID NO: 11, HCDR2 shown in SEQ ID NO: 12 and HCDR3 shown in SEQ ID NO: 13; LCDR1 shown in SEQ ID NO: 14, LCDR2 shown in SEQ ID NO: 15 and LCDR3 shown in SEQ ID NO: 16; or HCDR1 shown in SEQ ID NO: 11, HCDR2 shown in SEQ ID NO: 12 and HCDR3 shown in SEQ ID NO: 17; LCDR1 shown in SEQ ID NO:14, LCDR2 shown in SEQ ID NO:15 and LCDR3 shown in SEQ ID NO:18.

In another aspect, the application provides an antibody recognizing CD94, the antibody comprises a heavy chain variable region comprising the amino acid sequence shown in SEQ ID NO: 19 or 24, or a variant thereof.

In another aspect, the application provides an antibody recognizing CD94, the antibody comprises a light chain variable region comprising the amino acid sequence shown in SEQ ID NO: 21 or 26, or a variant thereof.

In another aspect, the present application provides an antibody recognizing CD94, comprising an antibody comprising the above-mentioned heavy chain variable region and light chain variable region.

Given that each of these heavy and light chain variable region sequences can bind CD94, the heavy and light chain variable region sequences can be “mixed and matched” to generate the anti-CD94 binding molecules of the present application.

In another aspect, the application provides variants of antibodies that bind CD94 or fragments thereof. The application thus provides antibodies or fragments thereof having heavy and/or light chain variable regions that are at least 80% identical in sequence to the variable region sequences of the heavy or light chains. Preferably, the amino acid sequence identity of the heavy and/or light chain variable regions is at least 85%, more preferably at least 90%, most preferably at least 95%, especially 96%, more particularly 97%, even more particularly 98%, most particularly 99%, including e.g. 80%, 81%, 82%, 83%, 84%, 85%, 86%, 87%, 88%, 89%, 90%, 91%, 92%, 93%, 94%, 95%, 96%, 97%, 98%, 99% and 100%. Variants can be obtained by using the antibody described in the present application as the parent antibody by yeast library screening, phage library screening, point mutation and other methods.

In another aspect, the application provides antibodies that specifically bind to CD94, the antibodies are whole antibodies, scFv, single domain antibodies, Fab fragments, Fab′ fragments, Fv fragments, F(ab′)₂ Fragments, Fd fragments, dAb fragments or multifunctional antibodies.

The present application also provides a nucleic acid molecule encoding the aforementioned antibody, preferably, the nucleic acid molecule of the present application is selected from SEQ ID NO: 20 or 25 encoding the heavy chain variable region, and/or selected from SEQ ID NO: 22 or 27 encoding the light chain variable region. More preferably, it is such a nucleic acid molecule comprising the heavy chain variable region sequence of SEQ ID NO:20, and comprising the light chain variable region sequence of SEQ ID NO:22; or comprising the heavy chain variable region sequence of SEQ ID NO:25, and comprising the light chain variable region sequence of SEQ ID NO:27.

In one embodiment, one or more vectors (e.g., expression vectors) comprising the above-described nucleic acids are provided.

The term “transplant immune rejection” refers to the immunological reaction that after the host carries out allograft transplantation of tissues, organs, or cells, the foreign graft is recognized by the host's immune system as an “alien component” and initiates attack, destruction and clearance against the graft. The present application provides an anti-transplant immune rejection cell and a method for anti-transplant rejection.

The term “graft” refers to a biological material or formulation derived from an individual other than the host for implantation into the host. The graft can be from any animal source, such as a mammalian source, preferably from a human. In some embodiments, the graft can be derived from the host, such as cells from the host that have been cultured in vitro, or engineered to be re-implanted into the host. In some embodiments, the graft can be from other allogeneic individual, such as cells from another human being cultured in vitro, or engineered into a host. In some embodiments, the graft may be from a xenogeneic individual, such as an organ from another species (e.g., murine, porcine, monkey) implanted into a human.

The term “cell” refers to cells of animal origin, human or non-human.

The term “host” refers to the recipient of the graft, which, in some embodiments, may be an individual, such as a human, to which exogenous cells are engrafted.

The term “individual” refers to any animal, such as a mammal or a marsupial. Individuals of the present application comprise, but are not limited to, humans, non-human primates (e.g., rhesus monkeys or other types of rhesus monkeys), mice, pigs, horses, donkeys, cattle, sheep, rats, and poultry of any kind.

The term “immune effector cell” refers to a cell involved in an immune response that produces an immune effect, such as T cells, B cells, natural killer (NK) cells, natural killer T (NKT) cells, dendritic cells, CIK cells, macrophages, mast cells, etc. In some embodiments, the immune effector cells are T cells, NK cells, NKT cells. In some embodiments, the T cells can be autologous T cells, xenogeneic T cells, allogeneic T cells. In some embodiments, the NK cells may be autologous NK cells or allogeneic NK cells. The term “CIK cells”, that is, cytokine-induced killer (CIK) cells are a new type of immune active cells, CIK has strong proliferation ability, strong cytotoxicity, and has certain immune characteristics. Because this cell expresses both CD3 and CD56 membrane protein molecules, it is also called NK cell (natural killer cell)-like T lymphocyte, which has both strong antitumor activity of T lymphocyte and non-MHC restriction tumor-killing advantages of NK cell.

The term “engineered cells with immune effector cell function” refers to a cell or cell line without immune effector that has acquired immune effector cell function after being engineered or stimulated by a stimulus. For example, 293T cells are engineered to acquire the function of immune effector cells; for example, stem cells are induced in vitro to differentiate into immune effector cells.

The “T cells” described herein can be PBMC, bone marrow, lymph node tissue, umbilical cord blood, thymus tissue and natural T cells obtained from infection sites, ascites, pleural effusion, spleen tissue, tumor tissue, and can also be a population of cells with specific phenotypic characteristics obtained by sorting, etc., or a mixed population of cells with different phenotypic characteristics, such as “T cells” can be cells comprising at least one subset of T cells: stem cell-like memory T cells (Tscm cells), central memory T cells (Tcm), effector T cells (Tef, Teff), regulatory T cells (tregs) and/or effector memory T cells (Tem). In some cases, a “T cell” may be T cell of a particular subtype, such as γδ T cells. In certain cases, T cells can be obtained from blood collected from an individual using any technique known to those skilled in the art, such as Ficoll™ separation and/or apheresis. T cells can be of any type and of any developmental stage, including but not limited to CD4+/CD8+ T cells, CD4+ helper T cells such as Th1 and Th2 cells, CD8+ T cells (e.g. cytotoxic T cells), tumor infiltrating cells, memory T cells, naïve T cells, etc. The T cells may be CD8+ T cells or CD4+ T cells. In one embodiment, cells from the circulating blood of an individual are obtained by apheresis. Apheresis products typically comprise lymphocytes, including T cells, monocytes, granulocytes, B cells, other nucleated leukocytes, red blood cells, and platelets. In one embodiment, cells collected by apheresis can be washed to remove plasma molecules and placed in a suitable buffer or medium for subsequent processing steps. In one embodiment, the T cells may be derived from a healthy donor, or from an individual diagnosed with cancer.

The terms “activation” and “activate” are used interchangeably and can refer to the process by which cells change from a resting state to an active state. This process may comprise responses to phenotypic or genetic changes in antigenic, migratory and/or functionally active states. For example, the term “activation” can refer to the process of stepwise activation of T cells. The activation process is co-regulated by the first stimulatory signal and the co-stimulatory signal. Activation of T cells is a dynamic process, and its duration and degree of activation are affected by external stimulation. “T cell activation” refers to the state of T cells that are stimulated to induce detectable cell proliferation, cytokine production, and/or detectable effector function. Using CD3/CD28 magnetic beads, antigen stimulation in vitro or in vivo will affect the degree and duration of T cell activation. In one embodiment, the engineered T cells are activated by co-incubation with tumor cells comprising a specific target antigen or after viral infection.

The term “peripheral blood mononuclear cell” (PBMC) refers to cells with a single nucleus in peripheral blood, including lymphocytes, monocytes, and the like.

The term “pluripotent stem cell” has the potential to differentiate into any one of the three germ layers: endoderm (e.g., gastric junction, gastrointestinal tract, lung, etc.), mesoderm (e.g., muscle, bone, blood, urogenital tissue, etc.) or ectoderm (e.g., epidermal tissue and nervous system tissue). As used herein, the term “pluripotent stem cell” also encompasses “induced pluripotent stem cell” or “iPSC,” which is a type of pluripotent stem cell derived from a non-pluripotent cell. In one embodiment, the pluripotent stem cells are derived from cells having the characteristics of pluripotent stem cells by reprogramming somatic cells. Such “iPS” or “iPSC” cells can be generated by inducing the expression of certain regulatory genes or by exogenously applying certain proteins.

The term “engineering” refers to a comprehensive science and technology that uses the principles and methods of cell biology and molecular biology to change the genetic material in cells or obtain cell products at the overall level of cells or at the level of organelles according to people's wishes through some engineering means. In one embodiment, the engineering refers to one or more alterations of nucleic acids, such as nucleic acids within the genome of an organism. In one embodiment, the engineering refers to changes, additions and/or deletions of genes. In one embodiment, the engineered cell may also refer to a cell with added, deleted and/or altered genes.

The terms “genetic modification”, “genetic modifying”, “genetically engineered” or “modified” refer to methods of modifying cells, including but not limited to cause gene defects: by means of gene editing, in the coding or non-coding regions of genes or their expression regulatory regions; or by endonuclease and/or antisense RNA technology; or by increasing changing of the protein expression level of the gene through introduction of exogenous proteins and/or complexes, small molecule inhibitors. In some embodiments, the modified cells are stem cells (e.g., hematopoietic stem cells (HSC) or progenitor cells, embryonic stem cells (ES), induced pluripotent stem (iPS) cells), lymphocytes (e.g., T cells), which can be obtained from the subject or donor. Cells can be modified to express foreign constructs, such as chimeric antigen receptors (CARs) or T cell receptors (TCRs), which can be integrated into the cell genome.

The term “gene silencing” refers to the phenomenon that a gene is not expressed or is underexpressed due to various reasons. Gene silencing can be gene silencing at the transcriptional level due to DNA methylation, heterochromatinization, and position effects; it can also be post-transcriptional gene silencing, that is, gene inactivation caused by specific inhibition of target RNA at the post-transcriptional level, including antisense RNA, co-suppression, gene repression, RNA interference, and microRNA-mediated translation inhibition, etc.; and can also be undetectable or low protein expression caused by increasing protein degradation, including PROTAC, LYTAC, AbTAC, ATTEC, AUTAC and intracellular retention of membrane proteins, etc.

The “TCR silencing” refers to no or low expression of endogenous TCR.

The “MHC silencing” refers to no or low expression of endogenous MHC.

“Low expression” as used herein means that the protein and/or RNA level of the target gene expressed in the engineered cell is lower than the expression level before the cell was engineerly treated. In specific embodiments, low expression of B2M or TCR or CD94 refers to a decrease in the expression of B2M or TCR or CD94 in cells by at least 1%, at least 5%, at least 10%, at least 20%, at least 30%, at least 40%, at least 50%, at least 60%, at least 70%, at least 80%, at least 90%, at least 95%, at least 99% or 100%. Protein expression or content in cells can be determined by any suitable method known in the art, such as ELISA, immunohistochemistry, Western Blotting or flow cytometry using antibodies specific for B2M or TCR or CD94. The term “MHC” stands for the major histocompatibility complex and is the collective name for all the gene groups that encode the antigens of the biocompatibility complex. MHC antigens are expressed in the tissues of all higher vertebrates, and are called HLA antigens in human cells and play an important role in transplantation reactions. Rejection is mediated by T cells that respond to histocompatibility antigens on the surface of the implanted tissue. MHC antigens are divided into NHC class I antigens and MHC class II antigens.

The term “human leukocyte antigen” (HLA) is the gene encoding the major histocompatibility complex in humans, located on chromosome 6 (6p21.31), and is closely related to the function of the human immune system. HLA comprises class I, class II and class III gene portions. The antigens expressed by HLA class I and class II genes are located on the cell membrane and are MHC-I (encoded by HLA-A, HLA-B, HLA-C sites) and MHC-II (encoded by HLA-D region), HLA Class I is distributed on the surface of almost all cells in the body and is a heterodimer composed of heavy chain (α chain) and β2 microglobulin (B2M). HLA Class II is mainly a glycoprotein located on the surface of macrophages and B lymphocytes.

The term “B2M” is beta-2 microglobulin, also known as B2M, is the light chain of an MHC class I molecule. In humans, B2M is encoded by the b2m gene located on chromosome 15, as opposed to other MHC genes as gene clusters localization on chromosome 6. Studies have shown that when the B2M gene is mutated, hematopoietic grafts from mice lacking normal cell surface MHC I expression are rejected by NK cells in normal mice, suggesting that defective expression of the MHC I molecule makes cells susceptible to rejection by the host immune system (Bix et al. 1991).

The term “T cell receptor (TCR)” mediates T cell recognition of specific major histocompatibility complex (MHC)-restricted peptide antigens, including classical TCR receptors and optimized TCR receptors. The classic TCR receptor is composed of two peptide chains, α and β. Each peptide chain can be divided into variable region (V region), constant region (C region), transmembrane domain and cytoplasmic region etc. The specificity exists in the V region, and the V region (Vα, Vβ) has three hypervariable regions, CDR1, CDR2, and CDR3. On the one hand, T cells expressing classical TCR can induce TCR specificity towards target antigens by using methods such as antigen stimulation on T cells. TCRs are divided into two categories: TCR1 and TCR2; TCR1 is composed of two chains, γ and δ, and TCR2 is composed of two chains, α and β. The term “TRAC” refers to the constant region of the TCRα chain.

The term “gene editing” refers to genetic engineering techniques that utilize site-specific nucleases to insert, knock out, modify or replace DNA at specific locations in the genome of an organism to alter DNA sequences. This technique is sometimes called “gene clipping” or “genome engineering.” Gene editing can be used to achieve precise and efficient gene knockout or gene knockin.

Nuclease-guided genome targeted modification technology usually consists of a DNA recognition domain and a non-specific endonuclease domain. The DNA recognition domain recognizes the target site and locates the nuclease to the genomic region that needs to be edited. Then, the double strands of DNA are cut by the non-specific endonuclease, causing the DNA breakage self-repair mechanism, thereby triggering the mutation of the gene sequence and promoting the occurrence of homologous recombination. The endonuclease may be a Meganuclease, a zinc finger nuclease, a CRISPR/Cas9 nuclease, a MBBBD-nuclease or a TALEN-nuclease. In a preferred embodiment, the endonuclease is CRISPR/Cas9 nuclease, TALEN-nuclease. Gene knockout technologies using nucleases comprise CRISPR/Cas9 technology, ZFN technology, TALE technology and TALE-CRISPR/Cas9 technology, Base Editor technology, guide editing technology and/or homing endonuclease technology.

The term “artificial zinc finger nucleases (ZFN)” technology is the first generation of nuclease site-directed modification technology, which uses zinc finger motifs that can specifically recognize triplet DNA fragments instead of bases as the basic unit of recognition of a specific DNA sequence. The most classic zinc finger nuclease is a fusion of a non-specific endonuclease FokI to a domain comprising a zinc finger, the purpose of which is naturally to cleave a specific sequence.

The term “transcription activator-like effector (TALE)” has DNA binding specificity, has a module that can specifically recognize bases, and is simple and convenient to operate. The TALE-DNA binding domain is composed of tandem repeat units, most of which comprise 34 amino acids. The 12th and 13th amino acids of the unit are designed as variable domains (repeat variable residues, RVD). The RVD of TALE recognizes the 4 bases of DNA sequence with high specificity, and the 13th amino acid directly binds specifically to the base(s) of DNA. According to the DNA sequence, a specific TALEDN recognition and binding domain can be constructed at any site, which can be widely used in gene sequence mutation modification and gene targeting. The DNA target sequence is set, the TALE-DNA binding domain is assembled, the non-specific DNA cleavage domain of Fok I endonuclease is fused, and the TALE nucleases (transcription activator-like effector nucleases, TALENs) are assembled. TALENs targetedly bind to DNA to generate DNA double-strand breaks (DSBs).

CRISPR/Cas9 is the third generation of gene editing technology. An example of this application uses CRISPR/Cas9 technology to prepare UCAR-T cells. The term “CRISPR (Clustered regularly interspaced short palindromicrepeats)” refers to clustered regularly interspaced short palindromic repeats. The term “Cas9 (CRISPR associated nuclease)” is a CRISPR-associated nuclease, an RNA-guided technology that uses Cas9 nuclease to edit targeted genes. Cas9 enzymes can be wild-type Cas9 or engineered Cas9. The “CRISPER/Cas9 system” collectively refers to transcripts and other elements involved in the expression of the Cas9 enzyme gene or guiding its activity, including sequences encoding the Cas9 gene, tracr (transactivating CRISPR) sequences (e.g., tracrRNA or active part tracrRNA), tracr paired sequences (covering “direct repeats” and partial direct repeats of tracrRNA processing in the context of endogenous CRISPR systems), guide sequences (also known as “spacers” in the context of endogenous CRISPR systems, i.e., gRNAs), or other sequences and transcripts from CRISPR loci. CRISPR systems are characterized by elements that facilitate the formation of a CRISPR complex (also referred to as a protospacer in the context of an endogenous CRISPR system) at the site of a target sequence. In the context of CRISPR complex formation, “target sequence” refers to a sequence to which a guide sequence is designed to be complementary, wherein hybridization between the target sequence and the guide sequence facilitates the formation of the CRISPR complex. Perfect complementarity is not required, provided that there is sufficient complementarity to cause hybridization and facilitate the formation of a CRISPR complex. After the CRISPR complex is formed, under the action of the cas9 enzyme, it can cut specific sites in the genome to introduce gene mutations; it can also regulate gene expression, such as activation or inhibition. A target sequence can comprise any polynucleotide, such as a DNA or RNA polynucleotide. In some embodiments, the target sequence is located in the nucleus or cytoplasm of the cell.

In general, a guide sequence (gRNA) is any polynucleotide sequence that is sufficiently complementary to a target polynucleotide sequence to hybridize to the target sequence and guide sequence-specific binding of the CRISPR complex to the target sequence. When the application involves the sequence of gRNA, it can be a targeted DNA sequence, or it can also be a complete Cas9 guide sequence formed by the ribonucleotides corresponding to the DNA, crRNA and TracrRNA. gRNAs are used to guide, bind or recognize Cas enzymes. In some embodiments, the degree of complementarity between the guide sequence and its corresponding target sequence is about or more than about 50%, 60%, 75%, 80%, 85%, 90%, 95%, 97.5%, 99% or more when optimally aligned using a suitable alignment algorithm. Optimal alignment can be determined using any suitable algorithm for aligning sequences, non-limiting examples of which comprise the Smith-Waterman algorithm, the Needleman-Wunsch algorithm, Algorithms based on the Burrows-Wheeler Transform (e.g., Burrows Wheeler Aligner), ClustalW, Clustal X, BLAT, Novoalign (Novocraft Technologies), ELAND Corporation (Illumina, San Diego, CA), SOAP (available at soap.genomics.org.cn), and Maq (available at maq.sourceforge.net). The term “sgRNA” refers to short gRNAs.

In some embodiments, the CRISPR enzyme is a part of a fusion protein that comprises one or more heterologous protein domains (e.g., about or more than about 1, 2, 3, 4, 5, 6, 7, 8, 9, 10 or more domains other than the CRISPR enzyme). The CRISPR enzyme fusion protein can comprise any other protein, and optionally a linker sequence between any two domains. Examples of protein domains that can be fused to CRISPR enzymes comprise, but are not limited to, epitope tags, reporter gene sequences, and one or more protein domains with the following activities: methylase activity, demethylase activity, transcription activation activity, transcriptional repression activity, transcriptional release factor activity, histone modification activity, RNA cleavage activity and nucleic acid binding activity. Non-limiting examples of epitope tags comprise histidine (His) tags, V5 tags, FLAG tags, influenza hemagglutinin (HA) tags, Myc tags, VSV-G tags, and thioredoxin (Trx) tags.

During gene editing, the administered gRNA, tracr paired sequence, and tracr sequence can be administered alone, or can be administered as a complete RNA sequence.

The combination of Cas9 protein and gRNA can cut DNA at a specific site. The CRISPR/Cas system derived from Streptococcus pyogenes recognizes sequence of 23 bp and can target 20 bp. The last 3 NGG sequence of its recognition site is called PAM (protospacer adjacent motif) sequence.

CRISPR/Cas transgenes can be delivered through vectors (e.g., AAV, adenovirus, lentivirus), and/or particles and/or nanoparticles, and/or electroporation.

This application simultaneously knocks out the genes TRAC and B2M to inactivate the functions of TCR and MHC molecules to obtain universal T cells or universal CAR-T cells. In one embodiment, the exons of the corresponding genes encoding the constant regions of one or both of the alpha and beta chains of B2M, TCR are knocked out using CRISPR/Cas technology, respectively. In one embodiment, the gRNA used for knocking out TCR is selected from the sequence shown in SEQ ID NO: 47, 48, 49, 50, 51, 52, 53 and/or 54. In one embodiment, the B2M gene in the engineered T cell is knocked out using CRISPR/Cas9 technology, and the gRNA used is selected from the sequences shown in SEQ ID NOs: 55, 56, 57 and/or 58.

In one embodiment, the cellular CD94 gene is knocked out. Exemplarily, the CD94 gene is knocked out using CRISPR/Cas9 technology, the gRNA used is selected from the sequence(s) shown in SEQ ID NO: 59, 60, 61, 62, 63, 64, 65, 66, 67, 68, 69, 70 and/or 71.

“Inhibiting” or “suppressing” the expression of B2M or TCR or CD94 means reducing the expression of B2M or TCR or CD94 in a cell by at least 1%, at least 5%, at least 10%, at least 20%, at least 30%, at least 40%, At least 50%, at least 60%, at least 70%, at least 80%, at least 90%, at least 95%, at least 99% or 100%. Protein expression or content in cells can be determined by any suitable method known in the art, such as ELISA, immunohistochemistry, Western Blotting or flow cytometry using antibodies specific for B2M or TCR or CD94.

The term “RNA interfering agent” is defined as any agent that interferes with or inhibits the expression of a target gene by RNA interference (RNAi). Such RNA interfering agents comprise, but are not limited to, nucleic acid molecules, short interfering RNAs (siRNAs), shRNAs, or miRNAs that are RNA molecules homologous to the target gene or fragments thereof, and small molecules that interfere with or inhibit the expression of the target gene by RNA interference (RNAi).

In an embodiment of the present application, specific CAR-T cells are constructed first, and then CRISPER/Cas9 technology is used to knock out the endogenous TRAC, B2M and/or CD94 of the CAR-T cells to construct the corresponding UCAR-T. In one embodiment, first CRISPER/Cas9 technology is used to knock out endogenous TRAC, B2M and/or CD94 to construct universal T cells, and then specific CAR is expressed to construct UCAR-T cells. In one embodiment, CRISPER/Cas9 technology is used to knock out endogenous TRAC, B2M and/or CD94 and express specific CAR simultaneously to construct UCAR-T cells.

The term “transfection” refers to the introduction of exogenous nucleic acid into a eukaryotic cell. Transfection can be achieved by various means known in the art, including calcium phosphate-DNA co-precipitation, DEAE-dextran-mediated transfection, polybrene-mediated transfection, electroporation, microinjection, liposome fusion, lipofection, protoplast fusion, retroviral infection and biolistics.

The terms “encoding nucleic acid molecule”, “encoding DNA sequence” and “encoding DNA” refer to the sequence of deoxyribonucleotides along a deoxyribonucleic acid chain. The sequence of these deoxyribonucleotides determines the sequence of amino acids along the polypeptide (protein) chain. Thus, a nucleic acid sequence encodes an amino acid sequence.

When used in reference to a nucleotide sequence, the term “sequence” as used herein may comprise DNA or RNA, and may be single-stranded or double-stranded.

The term sequence “identity” as used herein determines percent identity by comparing two best matched sequences over a comparison window (e.g., at least 20 positions), wherein the portion of the polynucleotide or polypeptide sequence in the comparison window may comprise additions or deletions (i.e., gaps), e.g., for the two best matched sequences, there is a gap of 20% or less (e.g., 5 to 15%, or 10 to 12%) compared to the reference sequence (which does not comprise additions or deletions). The percentage is usually calculated by determining the number of positions in the two sequences at which the same nucleic acid base or amino acid residue occurs, to yield the number of correctly matched positions, dividing the number of correctly matched positions by the total number of positions in the reference sequence (i.e., window size) and multiply the result by 100 to yield the percent of sequence identity.

The term “expression vector” as used herein refers to a vector comprising a recombinant polynucleotide, comprising expression control sequences operably linked to the nucleotide sequence to be expressed. The expression vector comprises sufficient cis-acting elements for expression; other elements for expression can be provided by the host cell or by an in vitro expression system. Expression vectors comprise all those known in the art, such as plasmids, viruses (e.g., lentiviruses, retroviruses, adenoviruses, and adeno-associated viruses).

The term “vector” as used herein is a composition that comprises an isolated nucleic acid and can be used to deliver the isolated nucleic acid to the interior of a cell. Numerous vectors are known in the art, including, but not limited to, linear polynucleotides, polynucleotides associated with ionic or amphiphilic compounds, plasmids, and viruses. Thus, the term “vector” comprises autonomously replicating plasmids or viruses. Non-plasmid and non-viral compounds that facilitate nucleic acid transfer into cells may also be comprised, such as polylysine compounds, liposomes, and the like.

The term “exogenous” refers to a nucleic acid molecule or polypeptide, cell, tissue, etc. that is not endogenously expressed in the organism itself, or the expression level is insufficient to achieve the function that it has when overexpressed.

The term “endogenous” refers to a nucleic acid molecule or polypeptide or the like that is derived from the organism itself.

The application provides T cells expressing a first protein comprising an antibody that recognizes CD94. In specific embodiments, the application also provides T cells expressing a first protein comprising a tandem antibody recognizing CD94 and a tumor antigen. In specific embodiments, the application also provides T cells expressing a first protein comprising a tandem antibody recognizing CD94 and a pathogen antigen.

The term “chimeric receptor” refers to a fusion molecule formed by linking DNA fragments or cDNA corresponding to protein from different sources by gene recombination technology, including extracellular domain, transmembrane domain and intracellular domain. Chimeric receptors comprise, but are not limited to: chimeric antigen receptor (CAR), chimeric T cell receptor, T cell antigen coupler (TAC).

The term “chimeric T cell receptor” consists of a TCR subunit combined with an antigen binding domain (such as an antibody domain), wherein the TCR subunit comprises at least part of the TCR extracellular domain, transmembrane domain, the stimulatory domain of the TCR intracellular signaling domain; the TCR subunit is effectively linked to the antibody domain. In specific embodiments, the extracellular, transmembrane, intracellular signaling domains of the TCR subunit are derived from CD3ε, CD3γ, CD3z, the alpha chain of TCR, or the beta chain of TCR, and the chimeric T cell receptor can form a complex with TCR/CD3 expressed on T cells.

The term “T cell antigen coupler (TAC)” comprises three functional domains: (1) antigen binding domain, including single chain antibody, designed ankyrin repeat protein (DARPin) or other targeting groups; (2) the extracellular domain, a single-chain antibody that binds to CD3, thereby bringing the TAC receptor close to the TCR receptor; (3) the transmembrane domain and the intracellular domain of CD4 co-receptor, where the intracellular domain is linked to the protein kinase LCK, catalyzes the phosphorylation of immunoreceptor tyrosine activation motifs (ITAMs) of the TCR complex as an initial step of T cell activation.

In some embodiments, the first protein of the present application is a chimeric antigen receptor (CAR).

The present application provides a T cell that can resist the killing of autologous or allogeneic NK cells. Specifically, the application provides T cells expressing CAR that recognizes CD94. The present application provides T cells expressing a CAR recognizing CD94 and with endogenous CD94 knockout. The application provides universal T cells expressing a CAR recognizing CD94 and with endogenous TCR and B2M knockouts. The present application provides universal T cells expressing a CAR recognizing CD94 and with endogenous CD94, TCR and B2M knockouts.

The present application also provides a T cell that can not only resist the killing of autologous or allogeneic NK cells, but can also significantly kill tumor cells. Specifically, the present application provides T cells expressing a CAR recognizing dual targets of CD94 and a tumor antigen. The present application provides T cells expressing a CAR recognizing dual targets of CD94 and pathogen antigens, and with endogenous CD94 knockout. The present application provides universal T cells expressing a CAR recognizing dual targets of CD94 and pathogen antigens, and with endogenous TCR and B2M knockouts. The present application provides universal T cells expressing a CAR recognizing dual targets of CD94 and pathogen antigens and with endogenous CD94, TCR and B2M knockouts.

The present application also provides a T cell that can not only resist the killing of autologous or allogeneic NK cells, but can also significantly kill tumor cells. In particular, the application provides T cells expressing a CAR recognizing CD94 and a CAR recognizing tumor antigens. The present application provides T cells expressing a CAR recognizing CD94 and a CAR recognizing tumor antigens and having endogenous CD94 knockout. The present application provides universal T cells expressing a CAR recognizing CD94 and a CAR recognizing tumor antigens and having endogenous TCR and B2M knockouts. The present application provides universal T cells expressing a CAR recognizing CD94 and a CAR recognizing tumor antigens and having endogenous CD94, TCR and B2M knockouts.

The present application also provides the combined use of the T cells that can resist autologous or allogeneic NK cell killing provided by the present application and T cells that express a second protein (such as CAR) that recognizes tumor antigens. The T cells that can resist the killing of autologous or allogeneic NK cells provided in this application can promote the survival of T cells expressing a second protein (such as CAR) that recognizes tumor antigens in the presence of autologous or allogeneic immune cells. In specific embodiments, the endogenous CD94 of the T cells expressing a second protein (such as CAR) that recognizes a tumor antigen is knocked out. In a specific embodiment, the T cells expressing a second protein (such as CAR) that recognizes tumor antigens are universal T cells in which endogenous TCR and B2M are knocked out. In a specific embodiment, the T cells expressing a second protein (such as CAR) that recognizes tumor antigens are universal T cells in which endogenous CD94, TCR and B2M are knocked out.

The term “chimeric antigen receptor” (CAR) comprises an extracellular antigen binding domain, a transmembrane domain, and an intracellular signaling domain. The intracellular signaling domain comprises a functional signaling domain of a stimulatory molecule and/or a costimulatory molecule; in one aspect, the stimulatory molecule is a zeta chain bound to a T cell receptor complex; in one aspect, a cytoplasmic signaling domain further comprises functional signaling domains of one or more costimulatory molecules, e.g., 4-1BB (i.e., CD137), CD27, and/or CD28. In certain embodiments, groups of polypeptides are linked to each other. Exemplarily, a CAR targeting CD94 comprises the sequence shown in SEQ ID NO: 43, 44, 45, or 46, and a CAR targeting CLDN18A2 comprises the sequence shown in SEQ ID NO: 74, 88, 89 or 90. The CAR targeting BCMA comprises the antigen binding domain shown in SEQ ID NO: 79, 99 or 100.

The term “primary signaling domain” modulates the initial activation of the TCR complex in a stimulatory manner. In one aspect, the primary signaling domain is initiated by, for example, binding of a TCR/CD3 complex to a peptide-loaded MHC molecule, thereby mediating T cell responses (including, but not limited to, proliferation, activation, differentiation, etc.). Primary signaling domains that act in a stimulatory manner may comprise immunoreceptor tyrosine activation motifs or signaling motifs of ITAM. Examples of ITAM-containing primary signaling domains that are particularly useful in this application comprise, but are not limited to, the sequences derived from TCRξ, FcRγ, FcRδ, CD3γ, CD3δ, CD3ε, CD5, CD22, CD79a, CD79b, CD278 (also known as “ICOS”) and CD66d, in a special case of the CAR of the present application, the intracellular signaling domain in any one or more of the CARs of the present application comprises an intracellular signaling sequence, e.g., the primary signaling domain of CD3ξ.

The term “signaling domain” refers to a functional portion of a protein that acts by transmitting messages within a cell to regulate cell activity via a defined signaling pathway by producing a second messenger or by acting as an effector in response to such a messenger. The intracellular signaling domain may comprise the entire intracellular portion of the molecule, or the entire native intracellular signaling domain, or functional fragments or derivatives thereof.

The term “costimulatory molecule” refers to a molecule that combines with a cell stimulatory signaling molecule, such as TCR/CD3, results in T cell proliferation and/or up- or down-regulation of key molecules. It is a cognate binding partner on a T cell that specifically binds a costimulatory ligand, thereby mediating a costimulatory response of the T cell, including but not limited to cell proliferation. Costimulatory molecules are non-antigen receptor cell surface molecules or their ligands that are required for an effective immune response. Costimulatory molecules include, but are not limited to, MHC class I molecules, receptors of BTLA and Toll ligand, and OX40, CD2, CD27, CD28, CDS, ICAM-1, LFA-1 (CD11a/CD18) and 4-1BB (CD137).

The intracellular signaling domain (or referred to as the domain) can be selected from any one of the costimulatory domains of Table 1. In some embodiments, the domains can be modified such that the identity to the reference domain can range from about 50% to about 100%. Any one of the domains of Table 1 can be modified such that the modified form can comprise about 50, 60, 70, 80, 90, 95, 96, 97, 98, 99, or up to about 100% identity.

TABLE 1
Costimulatory domains

| Gene marker | Abbreviation | Name |
|---|---|---|
| CD27 | CD27; T14; S152; Tp55; TNFRSF7; S152. | CD27 molecule |
| | LPFS2 | |
| CD28 | Tp44; CD28; CD28 antigent | CD28 molecule |
| TNFRSF9 | ILA; 4-1BB; CD137; CDw137 | Tumor Necrosis Factor Receptor |
| | | Superfamily Member 9 |
| TNFRSF4 | OX40; ACT35; CD134; IMD16; TXGP1L | Tumor Necrosis Factor Receptor |
| | | Superfamily Member 4 |
| TNFRSF8 | CD30; Ki-1; D1S166E | Tumor Necrosis Factor Receptor |
| | | Superfamily Member 8 |
| CD40LG | IGM; IMD3; TRAP; gp39; CD154; | CD40 ligand |
| | CD40L; HIGM1; T-BAM; TNFSF5; | |
| | hCD40L | |
| ICOS | AILIM; CD278; CVID1 | Inducible T cell co-stimulator |
| ITGB2 | LAD; CD18; MF17; MFI7; LCAMB; | Integrin β 2 (Complement |
| | LFA-1; MAC-1 | component 3 receptor 3 and 4 |
| | | subunits) |
| CD2 | T11; SRBC; LFA-2 | CD2 molecule |
| CD7 | GP40; TP41; Tp40; LEU-9 | CD7 molecule |
| KLRC2 | NKG2C; CD159c; NKG2-C | Killer cell Lectin like receptor |
| | | subfamily C, member 2 |
| TNFRSF18 | AITR; GITR; CD357; GITR-D | Tumor Necrosis Factor Receptor |
| | | Superfamily Member 18 |
| TNFRSF14 | TR2; ATAR; HVEA; HVEM; CD270; | Tumor Necrosis Factor Receptor |
| | LIGHTR | Superfamily Member 14 |
| HAVCR1 | TIM; KIM1; TIM1; CD365; HAVCR; | Hepatitis A virus cell receptor 1 |
| | KIM-1; TIM-1; TIMD1; TIMD-1; | |
| | HAVCR-1 | |
| LGALS9 | HUAT; LGALS9A, Galectin-9 | Human Galactose Lectin 9 |
| CD83 | BL11; HB15 | CD83 molecule |

In certain cases, the intracellular signaling domain can be designed to comprise several possible costimulatory signaling domains, it can comprise a single costimulatory domain, such as a zeta chain (the first generation CAR), or it can further comprise a costimulatory domain, such as zeta chain with CD28 or 4-1BB (the second generation CAR), or it may further comprise two costimulatory domains, such as zeta chain with CD28/OX40 or CD28/4-1BB (the third generation CAR). The signaling pathways used by these costimulatory molecules are all synergistic with the main T cell receptor activation signal. The signals provided by these costimulatory signaling domains can synergize with main effector activating signals derived from one or more ITAM motifs (e.g., the CD3zeta signaling domain) and can accomplish the requirements for T cell activation. Exemplarily, the signaling domain of CAR targeting CD94, CLDN18A2, and/or BCMA comprises CD3ζ. In a specific embodiment, the CD3ζ is a human CD3ζ molecule comprising the sequence shown in SEQ ID NO:41. Exemplarily, the signaling domain of CAR targeting CD94, CLDN18A2, and/or BCMA comprises the CD28 intracellular domain. In a specific embodiment, the CD28 intracellular domain comprises the sequence shown in SEQ ID NO:37.

The term “CD3ξ (also known as CD3 Zeta)” is defined as the protein provided by GenBan Accession No. BAG36664.1, or the equivalent residues from non-human species such as mouse, rodent, monkey, ape, etc. A “CD3 ξ domain” is defined as the amino acid residues from the cytoplasmic domain of the ξ chain, which is sufficient to functionally transmit the initial signals required for T cell activation. In one aspect, the cytoplasmic domain of ξ comprises residues 52 to 164 of GenBan Accession No. BAG36664.1, functional orthologs thereof—the equivalent residues from non-human species such as mouse, rodent, monkey, ape, etc. CD3ξ is also known as the T cell receptor T3ζ chain or CD247. This domain is part of the T cell receptor-CD3 complex and plays an important role in combining antigen recognition of several intracellular signaling pathways with primary effector activation of T cells. As used herein, CD3ζ refers primarily to human CD3ζ and its isoforms, as known from Swissprot entry P20963, including proteins having substantially the same sequence. As part of a chimeric antigen receptor, the full T-cell receptor T3ξ chain is not required, and any derivative thereof comprising the signaling domain of the T-cell receptor T3ξ chain is suitable, including any functional equivalents thereof “CD3ζ” is used interchangeably with “CD3z” and “CD3Z” in this application.

In the present application, in one aspect, the CAR comprises an extracellular antigen recognition domain, a transmembrane domain, and an intracellular signaling domain, wherein the intracellular signaling domain comprises a functional signaling domain derived from a stimulatory molecule. In one aspect, the CAR comprises an extracellular antigen recognition domain, a transmembrane domain, and an intracellular signaling domain, wherein the intracellular signaling domain comprises a functional signaling domain derived from a costimulatory molecule and the functional signaling domain derived from a stimulatory molecule. In one aspect, the CAR comprises an extracellular antigen recognition domain, a transmembrane domain, and an intracellular signaling domain, wherein the intracellular signaling domain comprises at least two functional signaling domains derived from one or more costimulatory molecules and functional signaling domain(s) derived from stimulatory molecule. In one aspect, the CAR comprises an optional leader sequence at the amino acid of the CAR protein. In one aspect, the CAR further comprises a leader sequence at the N-terminal of the extracellular antigen recognition domain, wherein the leader sequence is optionally cleaved from the antigen recognition domain (e.g., scFv) during cellular processing and localization of the CAR to the cell membrane. In one embodiment, the leader sequence comprises the sequence shown in SEQ ID NO:29.

A variety of antigen-binding regions can be designed for chimeric antigen receptors, including single-chain variable fragments (scFvs) derived from antibodies, fragments antigen-binding regions (Fabs) selected from libraries, single-domain fragments, or natural ligands that bind to their homologous receptors. In some embodiments, the extracellular antigen binding region may comprise scFv, Fab or natural ligands, and any derivatives thereof. An extracellular antigen binding region can refer to a molecule other than an intact antibody, which can comprise a portion of the intact antibody and can bind to the antigen to which the intact antibody binds. Examples of antibody fragments may comprise, but are not limited to, Fv, Fab, Fab′, Fab′-SH, F(ab′)2; diabodies, linear antibodies; single-chain antibody molecules (e.g., scFv); and multispecific antibodies formed by antibody fragments. Extracellular antigen binding regions, such as scFv, Fab or natural ligands, can be part of the CAR that determines antigen specificity. The extracellular antigen binding region can bind any complementary target. Extracellular antigen binding regions can be derived from antibodies of known variable region sequences. Extracellular antigen binding regions can be obtained from antibody sequences obtained from available mouse hybridomas. Alternatively, the extracellular antigen binding region can be obtained from whole exo-cleavage sequencing of tumor cells or primary cells such as tumor infiltrating lymphocytes (TILs).

In some embodiments, the binding specificity of the extracellular antigen binding region of the CAR can be determined by complementarity determining regions or CDRs, such as light chain CDRs or heavy chain CDRs. In many cases, binding specificity can be determined by the light and heavy chain CDRs. The present application provides T cells expressing CAR that recognizes CD94. In a specific embodiment, the application also provides T cells expressing CAR that recognizes dual targets of CD94 and tumor antigen. In a specific embodiment, the application also provides T cells expressing CAR that recognizes dual targets of CD94 and pathogen antigen.

In some embodiments, the extracellular antigen-binding region of the CAR comprises an antigen-binding region and a linker fragment (also referred to as a hinge, spacer, or linker). The linker fragment can be considered as part of a CAR used to provide flexibility to the extracellular antigen binding region. In some cases, the difference of linker fragments can lead to the CAR on the cell surface fail be activated by the target antigen, or to a very low degree of activation, or T cells will experience significant exhaustion and loss of function after activation, possibly due to the spatial structure formed by the antibody or antibody fragment of the extracellular antigen binding region is different in degree of complementarity with the spatial structure of the target antigen. For example, whether the length of an immunoglobulin-derived linker fragment needs to be optimized depends on the location of the target epitope targeted by the extracellular antigen binding region.

The composition and length of the linker fragment of the CAR polypeptide are adjustable. The spatial conformation of immune synaptic binding between T cells and target cells defines the distance at which membrane-distal epitopes on target molecules unable to be functionally bridged with CARs, and even CARs with short linking fragments cannot make the synaptic distance reach approximate value that the signal can be conducted. Likewise, The signal output of membrane-proximal CAR target epitopes were only observed in the context of long linker fragment CARs. Linker fragments can be adjusted depending on the extracellular antigen binding region used. Linker fragments can be of any length. Exemplarily, in one embodiment of the present application, the CAR comprises a hinge region, which is a CD8a hinge, preferably, the CD8a hinge region comprises the amino acids shown in SEQ ID NO:31.

The transmembrane (TM) domain (or region) of a CAR can anchor the CAR to the plasma membrane of cells. The natural transmembrane portion of CD28 can be used in CAR. In other cases, the natural transmembrane portion of CD8a can also be used in CAR. “CD8” may be a protein that is at least 85, 90, 95, 96, 97, 98, 99 or 100% identity with NCBI reference number: NP_001759 or a fragment thereof with stimulatory activity. A “CD8 nucleic acid molecule” can be a polynucleotide encoding a CD8 polypeptide, and in certain cases, the transmembrane domain can be the natural transmembrane portion of CD28, and “CD28” can refer to a protein that is at least 85, 90, 95, 96, 97, 98, 99 or 100% identity with NCBI reference number: NP_006130 or a fragment thereof with stimulatory activity. A “CD28 nucleic acid molecule” can be a polynucleotide encoding a CD28 polypeptide. In some embodiments, the transmembrane portion can comprise a CD8α region. Exemplarily, in one embodiment of the present application, the TM is a CD8 or CD28 transmembrane domain. In preferred embodiments, the TM is human CD8 transmembrane domain or human CD28 transmembrane domain. Preferably, the CD8 transmembrane domain comprises the amino acids of SEQ ID NO:33. The CD28 transmembrane domain comprises the amino acids of SEQ ID NO:35.

With regard to pharmaceutical compositions, the pharmaceutically acceptable carrier can be one of those conventionally used and is limited only by chemical physical considerations, such as solubility and non-reactivity with the active agent, and route of administration. The pharmaceutically acceptable carriers described herein, such as adjuvants, excipients and diluents, are well known to those skilled in the art and are readily available to the public. Preferably, a pharmaceutically acceptable carrier is one that is innocuous under the conditions of use and has no toxic side effects. There are a variety of suitable dosage forms for the pharmaceutical compositions of the present application. Methods of preparing administrable (e.g., parenterally) compositions are known or apparent to those skilled in the art.

The engineered cells of the present application can be administered to a subject in any suitable manner. Preferably, the CAR materials of the present application are administered by injection (e.g., subcutaneous, intravenous, intratumoral, intraarterial, intramuscular, intradermal, interperitoneal, or intrathecal). Preferably, the CAR material of the present application is administered intravenously. Suitable pharmaceutically acceptable carriers for injectable CAR materials of the present application can comprise any isotonic carrier, for example, physiological saline (about 0.90% w/v NaCl in water, about 300 mOsm/L NaCl in water, or about 9.0 g NaCl per liter of water), normal temperature or electrolyte solution. In one embodiment, the pharmaceutically acceptable carrier is supplemented with human serum protein.

An “effective amount” or “therapeutically effective amount” refers to a dose sufficient to prevent or treat a disease (cancer) in a subject. Effective doses for therapeutic or prophylactic use depend on the stage and severity of the disease being treated, the age, weight and general health state of the subject, and the judgment of the prescribing physician. The size of the dose will also depend on the active substance selected, the method of administration, the timing and frequency of administration, the presence, nature and extent of adverse side effects that may accompany administration of the particular active substance, and the desired physiological effect. According to the judgment of the prescribing physician or those skilled in the art, one or more rounds, or multiple administrations of the CAR materials of the present application may be required. By way of example and not limiting the present application, when the CAR material of the present application is a host cell, an exemplary dose of the host cell may be at least one million cells (1×10⁶ cells/dose).

Embodiments of the present application also comprise depletion of mammalian lymphocytes prior to administration of the CAR material of the present application, including but not limited to non-myeloablative lymphoid depletion chemotherapy, myeloablative lymphoid depletion chemotherapy, total body irradiation, and the like.

The term “treatment” refers to interventions that attempt to modify the disease process, either prophylactically or clinically. Therapeutic effects comprise, but are not limited to, preventing the occurrence or recurrence of the disease, alleviating symptoms, reducing any direct or indirect pathological consequences of disease, preventing metastasis, slowing the rate of disease progression, improving or relieving the condition, relieving or improving the prognosis, etc. In a specific embodiment, the engineered T cells provided in the present application can inhibit tumor cell proliferation, and/or inhibit tumor cell proliferation and increasement of tumor volume in vivo.

The term “prevention” refers to interventions that are attempted prior to the development of a disease such as rejection of a cell transplant.

The application provides the CAR, nucleic acid, recombinant expression vector, host cell, cell collection or pharmaceutical composition of the present application for use in the treatment or prevention of mammalian tumors.

The engineered T cells provided in this application can be used to treat, prevent or ameliorate autoimmune diseases or inflammatory diseases, especially inflammatory diseases associated with autoimmune diseases, such as arthritis (e.g., rheumatoid arthritis, arthritis chronica progrediente and deformed arthritis) and rheumatic diseases, including inflammatory conditions and rheumatic diseases involving bone loss and inflammatory pain, spondyloarthropathies (including ankylosing spondylitis), Reiter Syndrome, reactive arthritis, psoriatic arthritis, juvenile idiopathic and enteropathic arthritis, enthesitis, hypersensitivity reactions (including airway hypersensitivity and skin hypersensitivity) and allergies. The engineered T cells provided herein are used for the treatment and prevention of diseases comprising autoimmune hematological disorders (including, for example, hemolytic anemia, aplastic anemia, pure red cell anemia, and idiopathic thrombocytopenia), systemic lupus erythematosus (SLE), lupus nephritis, inflammatory muscle disease (dermatomyositis), periodontitis, polychondritis, scleroderma, Wegener's granulomatosis, dermatomyositis, chronic active hepatitis, myasthenia gravis, psoriasis, Stephen Johnson syndrome, spontaneous sprue, autoimmune inflammatory bowel disease (including, for example, ulcerative colitis, Crohn's disease, and irritable bowel syndrome), endocrine eye disease, Graves disease, Sarcoidosis, multiple sclerosis, systemic sclerosis, fibrotic diseases, primary biliary cirrhosis, juvenile diabetes (type I diabetes), uveitis, keratoconjunctivitis sicca and vernal keratoconjunctivitis, interstitial pulmonary fibrosis, periprosthetic osteolysis, glomerulonephritis (with and without nephrotic syndrome, including, for example, idiopathic nephrotic syndrome or minimal change nephropathy), multiple myeloma, other types of tumors, inflammatory diseases of the skin and cornea, myositis, loosening of bone implants, metabolic disorders (such as obesity, atherosclerosis and other cardiovascular diseases including dilated cardiomyopathy, myocarditis, type II diabetes and dyslipidemia) and autoimmuno thyrouiditis disease (including Hashimoto's thyroiditis), primary vasculitis of small and medium vessels, large vessel vasculitis including giant cell arteritis, hidradenitis suppurativa, neuromyelitis optica, Sjogren's syndrome, Behcet's Disease, atopic and contact dermatitis, bronchiolitis, inflammatory muscle disease, autoimmune peripheral neuropathy, immune kidney, liver and thyroid disease, inflammation and atherosclerosis, autoinflammatory fever syndrome, immunohematology disorders and bullous diseases of the skin and mucous membranes.

The engineered T cells provided in this application can be used to treat, prevent or ameliorate asthma, bronchitis, bronchiolitis, idiopathic interstitial pneumonia, pneumoconiosis, emphysema, and other obstructive or inflammatory diseases of the airways.

The engineered T cells of the present application can be used as the sole active ingredient or in combination with other drugs such as immunosuppressive or immunomodulatory agents or other anti-inflammatory or other cytotoxic or anticancer agents (e.g., as adjuvants thereof or in combination with them), for example, to treat or prevent diseases associated with immune disorders. For example, the antibodies of the present application can be used in combination with the following drugs: DMARDs, such as gold salts, sulfasalazine, antimalarial drugs, methotrexate, D-penicillamine, azathioprine, mycophenolic acid, tacrolimus, sirolimus, minocycline, leflunomide, glucocorticoids; calcineurin inhibitors such as cyclosporine A or FK 506; modulators of lymphocyte recycling such as FTY720 and FTY720 analogs; mTOR inhibitors such as rapamycin, 40-O-(2-hydroxyethyl)-rapamycin, CCI779, ABT578, AP23573 or TAFA-93; Ascomycetes with immunosuppressive properties such as ABT-281, ASM981, etc.; corticosteroids; cyclophosphamide; azathioprine; leflunomide; mizoribine; mycophenolate mofetil; 15-deoxyspergualin or its immunosuppressive homologs, analogs or derivatives; immunosuppressive monoclonal antibodies, e.g., against leukocyte receptors, such as MHC, CD2, CD3, CD4, CD7, CD8, CD25, CD28, CD40, CD45, CD58, CD80, CD86, or monoclonal antibodies of their ligands; other immunomodulatory compounds.

The tumor described herein can be any tumor, including acute lymphoblastic carcinoma, acute myeloid leukemia, alveolar rhabdomyosarcoma, bladder cancer, bone cancer, brain cancer (e.g., medulloblastoma), breast cancer, anal cancer, anal canal or anorectal cancer, eye cancer, intrahepatic bile duct cancer, joint cancer, neck cancer, gallbladder or pleura cancer, nose cancer, nasal cavity or middle ear cancer, oral cancer, vulvar cancer, chronic lymphocytic leukemia (CLL), chronic myeloid cell cancer, colon cancer, esophageal cancer, cervical cancer, fibrosarcoma, gastrointestinal carcinoid, head and neck cancer (such as head and neck squamous cell carcinoma), Hodgkin's lymphoma, hypopharyngeal cancer, kidney cancer, laryngeal cancer, leukemia, liver cancer, lung cancer (e.g., non-small cell lung cancer), lymphoma, malignant mesothelioma, mast cell tumor, melanoma, multiple myeloma, nasopharyngeal carcinoma, non-Hodgkin's lymphoma, B-chronic lymphocyte leukemia, B-precursor acute lymphoblastic leukemia (B-ALL), B-cell precursor acute lymphocytic leukemia (BCP-ALL), B-cell lymphoma, acute lymphocytic leukemia (ALL), Burkitt lymphoma, ovarian cancer, pancreatic cancer, pharyngeal cancer, prostate cancer, rectal cancer, kidney cancer, skin cancer, small intestine cancer, soft tissue cancer, stomach cancer, testicular cancer, thyroid cancer, ureteral cancer. Preferably, the tumor is characterized by BCMA expression, and more preferably is a multiple myeloma characterized by BCMA expression.

“Tumor antigen” refers to an antigen that is new or overexpressed during the development, progression of a hyperproliferative disease. In certain aspects, the hyperproliferative disorder of the present application refers to cancer.

The tumor antigens described in this application can be solid tumor antigens or hematological tumor antigens.

The tumor antigens of the present application comprise, but are not limited to: thyroid-stimulating hormone receptor (TSHR); CD171; CS-1; C-type lectin-like molecule-1; ganglioside GD3; Tn antigen; CD19; CD20; CD 30; CD 70; CD 123; CD 138; CD33; CD44; CD44v7/8; CD38; CD44v6; B7H3 (CD276), B7H6; KIT (CD117); interleukin-13 receptor subunit α (IL-13R α); interleukin 11 receptor α (IL-11Rα); prostate stem cell antigen (PSCA); prostate specific membrane antigen (PSMA); carcinoembryonic antigen (CEA); NY-ESO-1; HIV-1 Gag; MART-1; gp100; tyrosinase; mesothelin; EpCAM; protease serine 21 (PRSS21); vascular endothelial growth factor receptor, vascular endothelial growth factor receptor 2 (VEGFR2); lewis (y) antigen; CD24; platelet-derived growth factor receptor β (PDGFR-β); stage-specific embryonic antigen-4 (SSEA-4); cell surface associated mucin 1 (MUC1), MUC6; epidermal growth factor receptor family and its mutants (EGFR, EGFR2, ERBB3, ERBB4, EGFRvIII); neural cell adhesion molecule (NCAM); carbonic anhydrase IX (CAIX); LMP2; ephrin A receptor 2 (EphA2); fucosyl GM1; sialic Lewis adhesion molecule (sLe); ganglioside GM3; TGSS; high molecular weight melanoma-associated antigen (HMWMAA); o-acetyl GD2 ganglioside (OAcGD2); folate receptor; tumor vascular endothelial marker 1 (TEM1/CD248); tumor vascular endothelial marker 7-related (TEM7R); Claudin 6, Claudin18.2, Claudin18.1; ASGPR1; CDH16; 5T4; 8H9; avr36 integrin; B cell maturation antigen (BCMA); CA9; kappa light chain; CSPG4; EGP2, EGP40; FAP; FAR; FBP; embryonic AchR; HLA-A1, HLA-A2; MAGEA1, MAGE3; KDR; MCSP; NKG2D ligand; PSC1; ROR1; Sp17; SURVIVIN; TAG72; TEM1; fibronectin; tenascin; carcinoembryonic variant of tumor necrosis region; G protein-coupled receptor class C group 5-member D (GPRCSD); X chromosome open reading frame 61 (CXORF61); CD97; CD179a; anaplastic lymphoma kinase (ALK); polysialic acid; placenta-specific 1 (PLAC1); hexasaccharide portion of of globoH glycoceramide (GloboH); mammary gland differentiation antigen (NY-BR-1); uroplakin 2 (UPK2); hepatitis A virus cell receptor 1 (HAVCR1); adrenergic receptor 03 (ADRB3); pannexin 3 (PANX3); G protein-coupled receptor 20 (GPR20); lymphocyte antigen 6 complex locus K9 (LY6K); olfactory receptor 51E2 (OR51E2); TCRγ alternating reading frame protein (TARP); Wilms tumor protein (WT1); ETS translocation variant 6 (ETV6-AML); sperm protein 17 (SPA17); X antigen family member 1A (XAGE1); angiopoietin-binding cell surface receptor 2 (Tie2); melanoma cancer testis antigen-1 (MAD-CT-1); melanoma cancer testis antigen-2 (MAD-CT-2); Fos-associated antigen 1; p53 mutant; human telomerase reverse transcriptase (hTERT); sarcoma translocation breakpoint; cell melanoma inhibitor of apoptosis (ML-IAP); ERG (transmembrane protease serine 2 (TMPRSS2) ETS fusion gene); N-acetylglucosaminyltransferase V (NA17); paired box protein Pax-3 (PAX3); androgen receptor; cyclin B1; V-myc avian myelocytomatosis viral neuroblastoma derived homolog (MYCN); Ras homolog family member C (RhoC); cytochrome P450 1B1 (CYP1B1); CCCTC-binding factor (zinc finger protein)-like (BORIS); squamous cell carcinoma antigen 3 (SART3) recognized by T cells; paired box protein Pax-5 (PAX5); proacrosin-binding protein sp32 (OYTES1); lymphocyte-specific protein tyrosine kinase (LCK); A-kinase-anchored protein 4 (AKAP-4); synovial sarcoma X breakpoint 2 (SSX2); CD79a; CD79b; CD72; leukocyte-associated immunoglobulin-like receptor 1 (LAIR1); Fc fragment of IgA receptor (FCAR); leukocyte immunoglobulin-like receptor subfamily member 2 (LILRA2); CD300 molecule-like family member f (CD300LF); C-type lectin domain family 12 member A (CLEC12A); bone marrow stromal cell antigen 2 (BST2); EGF-like module containing mucin-like hormone receptor-like 2 (EMR2); lymphocyte antigen 75 (LY75); glypican-3 (GPC3); Fc receptor-like 5 (FCRL5); immunoglobulin lambda-like polypeptide 1 (IGLL1). Preferably, the tumor antigen is Claudin18.2, GPC3, BCMA or CD19.

The pathogen antigens are selected from: antigens of viruses, bacteria, fungi, protozoa, or parasites; the viral antigens are selected from: cytomegalovirus antigens, Epstein-Barr virus antigens, human immunodeficiency virus antigens, or influenza virus antigens.

Provided herein is an autologous or allogeneic immune cell (e.g., T cell) that is genetically engineered to express a CD94-CAR for resisting killing of NK cells, thereby providing a method for increasing the persistence and/or transplant survival rate of autologous or allogeneic first immune cells in the presence of secondary immune cells of the host. For clarity, the “host” is the recipient of the “first immune cell”, such as a subject, a patient, etc.; after the “first immune cell” has been engineered and transplanted into the host, any one of the other immune cells other than the “first immune cell” is referred to as the “second immune cell”. The “first immune cell” and the “second immune cell” may be cells from the same individual, or may be allogeneic cells.

In some embodiments, the first immune cell is genetically engineered to express CD94-CAR. In some embodiments, the first immune cell is genetically engineered to express CD94-CAR, and gene editing techniques are used to knock out endogenous CD94. In some embodiments, the first immune cell is genetically engineered to express CD94-CAR, and gene editing techniques are used to knock out endogenous B2M and TCR. In some embodiments, the first immune cell is genetically engineered to express CD94-CAR, and gene editing techniques are used to knock out endogenous CD94, B2M, and TCR.

In some embodiments, the first immune cell is genetically engineered to express CD94-CAR, the cell is also genetically engineered to express at least one non-CD94-targeting chimeric receptor (CAR, modified TCR, TFP, TAC, aTCR, or a combination thereof). In some embodiments, the first immune cell is genetically engineered to express CD94-CAR, the cell is also genetically engineered to express at least one non-CD94-targeting chimeric receptor (CAR, modified TCR, TFP, TAC, aTCR, or a combination thereof), and gene editing technology is used to knock out endogenous CD94. In some embodiments, the first immune cell is genetically engineered to express a CD94-CAR, the cell is also genetically engineered to express at least one non-CD94-targeting chimeric receptor (CAR, modified TCR, TFP, TAC, aTCR, or a combination thereof), and gene editing technology is used to knock out endogenous B2M and TCR. In some embodiments, the first immune cell is genetically engineered to express a CD94-CAR, the cell is also genetically engineered to express at least one non-CD94-targeting chimeric receptor (CAR, modified TCR, TFP, TAC, aTCR, or a combination thereof), and gene editing technology is used to knock out endogenous CD94, B2M and TCR.

In some embodiments, the first immune cell is genetically engineered to express a CAR that recognizes CD94 and a tumor antigen dual-targets. In some embodiments, the first immune cell is genetically engineered to express a CAR that recognizes CD94 and a tumor antigen dual-targets, and gene editing technology is used to knock out endogenous CD94. In some embodiments, the first immune cell is genetically engineered to express a CAR that recognizes CD94 and a tumor antigen dual-targets, and gene editing technology is used to knock out endogenous B2M and TCR. In some embodiments, the first immune cell is genetically engineered to express a CAR that recognizes CD94 and a tumor antigen dual-targets, and gene editing technology is used to knock out endogenous CD94, B2M, and TCR.

This application includes, for example, those CAR-T cells and their preparation methods disclosed in the Chinese application publication number: CN107058354A, CN107460201A, CN105194661A, CN105315375A, CN105713881A, CN106146666A, CN106519037A, CN106554414A, CN105331585A, CN106397593A, CN106467573A, CN104140974A, CN 108884459 A, CN107893052A, CN108866003A, CN108853144A, CN109385403A, CN109385400A, CN109468279A, CN109503715A, CN 109908176 A, CN109880803A, CN 110055275 A, CN110123837A, CN 110438082 A, CN 110468105 A and the international application publication number: WO2017186121A1, WO2018006882A1, WO2015172339A8, WO2018/018958A1, WO2014180306 A1, WO2015197016A1, WO2016008405A1, WO2016086813A1, WO2016150400A1, WO2017032293A1, WO2017080377A1, WO2017186121A1, WO2018045811A1, WO2018108106A1, WO 2018/219299, WO2018/210279, WO2019/024933, WO2019/114751, WO2019/114762, WO2019/141270, WO2019/149279, WO2019/170147A1, WO 2019/210863, WO2019/21902.

### EXAMPLES

The present application will be further described below with reference to specific embodiments. It should be understood that these examples are only used to illustrate the present application and not to limit the scope of the present application. The experimental methods that are not indicated with specific conditions in the following examples are usually in accordance with conventional conditions such as those described in J. Sambrook et al., Molecular Cloning Experiment Guide, 3rd Edition, Science Press, 2002, or according to the proposed conditions described by the manufacturer.

### Example 1. Preparation of Antigens

Human CD94 gene was extracted from NK cells, the NKG2A gene (Cat: HG13905-G) was purchased from Yiqiao Shenzhou, and the eukaryotic expression plasmids V152S-huFc(knob)-NKG2A-ECD and V152S-huFc(hole)-CD94-ECD were respectively constructed using the knob into hole technology, wherein CD94 and NKG2A are fused and expressed at the C-terminus of the Fc segment of human IgG1. The sequence information is as follows: NKG2A extracellular segment (SEQ ID NO:5), CD94 extracellular segment (SEQ ID NO:3), huFc(knob) (SEQ ID NO:7) and huFc(hole) (SEQ ID NO:9).

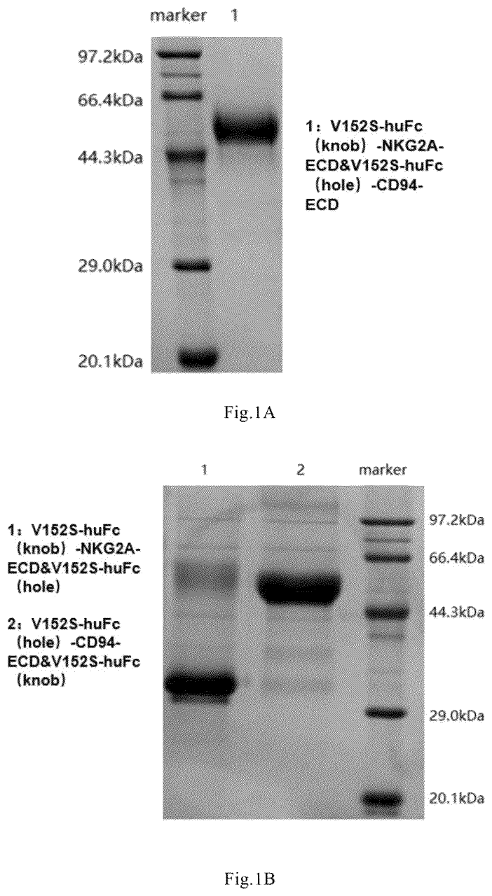

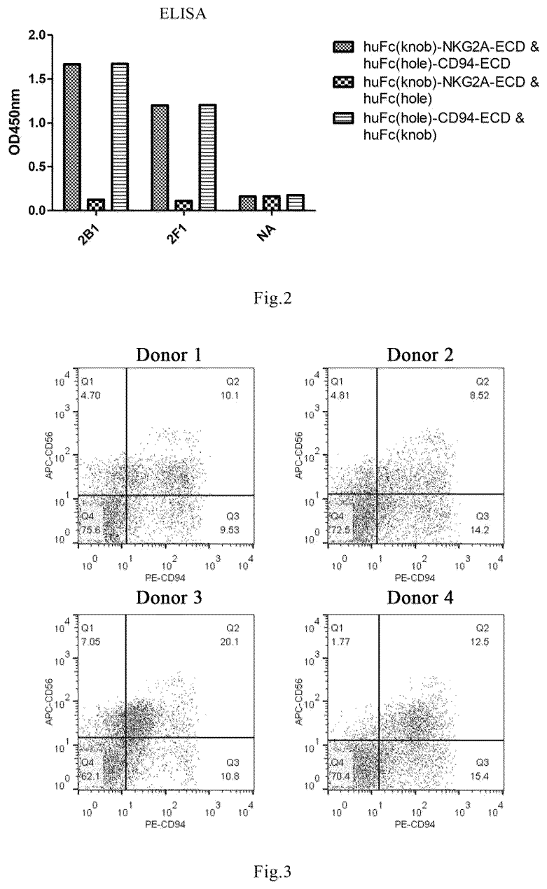

By transfecting HEK293 cells, the heterodimeric recombinant proteins of CD94 and NKG2A, the NKG2A monomer Fc fusion protein and the CD94 monomeric Fc fusion protein were expressed, respectively. The transfected cells were cultured for 7 days. The culture medium was centrifuged, took the supernatant, and the antigens huFc(knob)-NKG2A-ECD and huFc(hole)-CD94-ECD (FIG. 1A-1), huFc(knob)-NKG2A-ECD and huFc(hole) (FIG. 1B-1), huFc(hole)-CD94-ECD and huFc(knob) (FIG. 1B-2) were obtained by affinity purification with Mabselect Sure Column.

The purified product was detected by electrophoresis, and the purity was more than 90%. The electrophoresis result is shown in FIG. 1 below.

### Example 2. Discovery and Identification of CD94 Antibodies

The phage display library used in this application was a phage library constructed by our company, and the library capacity was 1E+11. Fab fragments that specifically bind to CD94 were obtained by screening methods known to those skilled in the art.

Briefly, immunotubes were coated with 10 μg/ml of the antigene CD94 monomeric Fc fusion protein prepared above, blocked with phosphate buffered saline (MPBS) comprising 3% bovine serum albumin (BSA) for 2 hours at room temperature. In order to screen for antibodies that specifically bind to CD94, the phage library was added to the immunotubes coated with the antigen CD94 monomeric Fc fusion protein and bound for 1.5 hours. The non-specific phages were then washed away, and the bound phages were eluted with 100 mM triethylamine and infected E. coli TG1 in logarithmic growth phase. Expanded culture of the eluted phages, and the expanded phage library was purified using PEG/NaCl precipitation for the next round of screening.

A total of three rounds of screening were performed, and 9 positive clones that specifically bind to CD94 were obtained after three rounds of screening. These clones were subsequently subjected to ELISA and sequencing analysis, two unique sequences were selected, 2B1 and 2F1, respectively. The ELISA results are shown in FIG. 2 (NA is the blank control group).

For 2B1, the amino acid sequence of HCDR1 is shown in SEQ ID NO:11, the amino acid sequence of HCDR2 is shown in SEQ ID NO:12, the amino acid sequence of HCDR3 is shown in SEQ ID NO:13, and the amino acid sequence of LCDR1 is shown in SEQ ID NO:14, the amino acid sequence of LCDR2 is shown in SEQ ID NO: 15, and the amino acid sequence of LCDR3 is shown in SEQ ID NO: 16. For 2B1, the amino acid sequence of the heavy chain variable region is shown in SEQ ID NO:19, and the amino acid sequence of the light chain variable region is shown in SEQ ID NO:21.

For 2F1, the amino acid sequence of HCDR1 is shown in SEQ ID NO:11, the amino acid sequence of HCDR2 is shown in SEQ ID NO:12, the amino acid sequence of HCDR3 is shown in SEQ ID NO:17, and the amino acid sequence of LCDR1 is shown in SEQ ID NO:14, the amino acid sequence of LCDR2 is shown in SEQ ID NO: 15, and the amino acid sequence of LCDR3 is shown in SEQ ID NO: 18. For 2F1, the amino acid sequence of the heavy chain variable region is shown in SEQ ID NO:24, and the amino acid sequence of the light chain variable region is shown in SEQ ID NO:26.

### Example 3. Expression of CD94 on the Surface of NK Cells, T Cells, B Cells and Monocytes

Using Ficoll-Paque (GE bioscience) for density gradient centrifugation, PBMCs were isolated from peripheral blood of 4 donors, and NK cells were isolated using NK cell isolation kit (purchased from Miltenyi). The expression of CD94 on the surface of NK cells (CD56+ cells) was detected by APC-CD56 antibody (Invitrogen) and PE-CD94 antibody (Invitrogen).

The results of flow cytometry are shown in FIG. 3. CD94 was expressed on all CD56+ cells, and there were CD94^{low} and CD94^{high} two subgroups.

Ficoll-Paque (GE bioscience) was used for density gradient centrifugation, PBMCs were isolated from peripheral blood of 5 donors, and NK cells in PBMC cells were isolated using NK cell isolation kit (purchased from Miltenyi). IL-2 (500 U/ml) and IL-15 (150 U/ml) were added to the NK cell culture medium and incubated for 10 days. CD94+NK cells were detected by flow cytometry with PE-CD94 (Invitrogen) antibody.

The results of flow cytometry are shown in FIG. 4, and CD94 was expressed on more than 90% of activated NK cells.

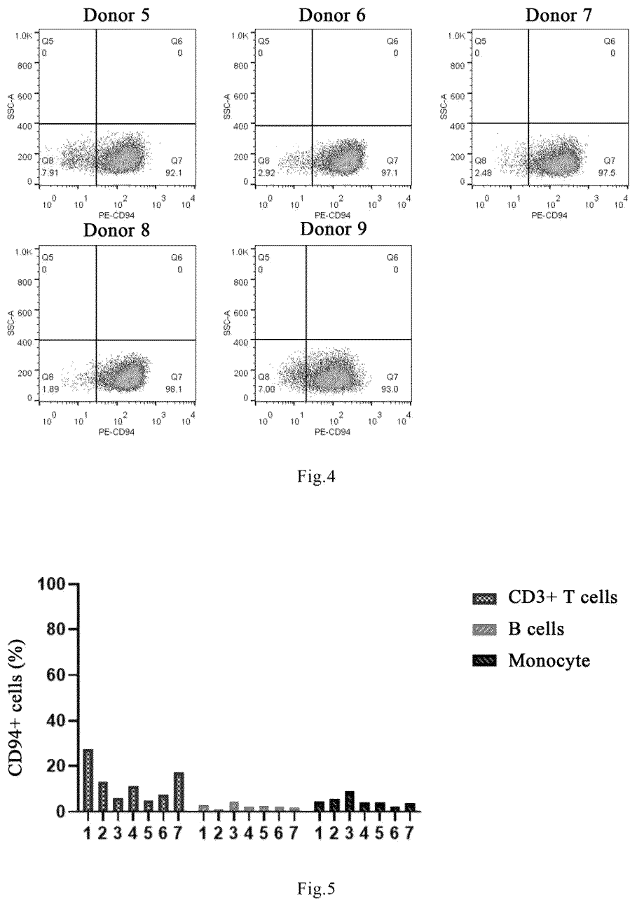

At the same time, we also detected the expression of CD94 on T cells, B cells and monocytes from 7 different donors (FIG. 5). The results showed that CD94 was expressed on an average of 12% of T cells, 2% of B cells, and 4.8% of monocytes, with low expression ratios.

In conclusion, CD94 was mainly expressed on resting and more than 90% of activated NK cells, and on a small proportion of T cells.

### Example 4. Preparation of CAR-T Cells

1. Preparation of CD94 CAR-T Cells

In this example, CD94 was selected as the target of CAR-T cells, and the preparation method was operated according to the conventional CAR-T cell preparation method in the field.

The scFv used in this example was the antibody 2F1 or 2B1 targeting human CD94, and the fragments shown in Table 2 were inserted respectively for construction of CAR-T cells.

TABLE 2
Chimeric Antigen Receptors

| | Signal peptide - extracellular binding region - hinge region - |
|---|---|
| Chimeric antigen receptors | transmembrane domain - intracellular signaling domain |
| 2F1-28Z | CD8α signal peptide -ScFv(2F1)- CD8α hinge region - CD28 |
| | transmembrane domain and CD28 intracellular signaling region - |
| | CD3ζ intracellular signaling region |
| 2F1-BBZ | CD8α signal peptide -ScFv(2F1)- CD8α hinge region - CD8 |
| | transmembrane domain - CD137 intracellular signaling region - |
| | CD3ζ intracellular signaling region |
| 2B1-28Z | CD8α signal peptide -ScFv(2B1)- CD8α hinge region - CD28 |
| | transmembrane domain and CD28 intracellular signaling region - |
| | CD3ζ intracellular signaling region |
| 2B1-BBZ | CD8α signal peptide -ScFv(2B1)- CD8α hinge region - CD8 |
| | transmembrane domain - CD137 intracellular signaling region - |
| | CD3ζ intracellular signaling region |

The sequence of scFv(2B1) is shown in SEQ ID NO: 23, the sequence of scFv(2F1) is shown in SEQ ID NO: 28, the sequence of CD8α signal peptide is shown in SEQ ID NO: 29, and the sequence of CD8α hinge region is shown in SEQ ID NO: 31, the sequence of the CD8 transmembrane domain is shown in SEQ ID NO: 33, the sequence of the CD28 transmembrane domain is shown in SEQ ID NO: 35, and the sequence of the CD28 intracellular domain is shown in SEQ ID NO: 37, the sequence of the CD137 intracellular domain is shown in SEQ ID NO:39, and the sequence of CD3 is shown in SEQ ID NO:41.

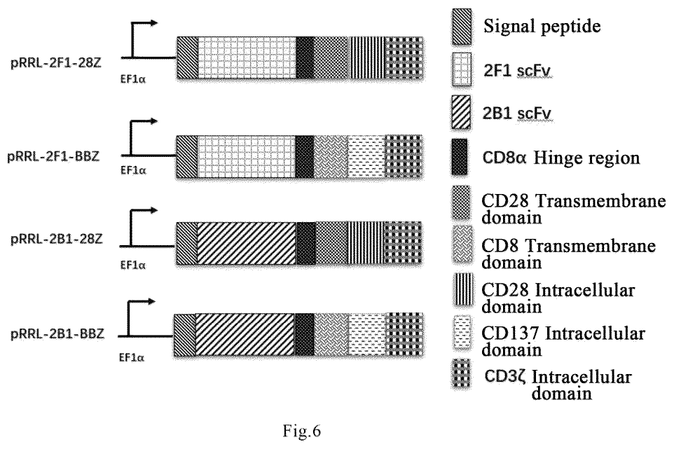

The nucleic acid sequence of 2F1-28Z, the nucleic acid sequence of 2F1-BBZ, the nucleic acid sequence of 2B1-28Z, and the nucleic acid sequence of 2B1-BBZ were inserted into the lentiviral vector to construct lentiviral plasmids PRRL-2F1-28Z, PRRL-2F1-BBZ, PRRL-2B1-28Z, PRRL-2B1-BBZ (see FIG. 6), and then transfected into 293T cells, respectively, and packaged to obtain lentiviruses 2F1-28Z, 2F1-BBZ, 2B1-28Z, and 2B1-BBZ.

Density gradient centrifugation was performed with Ficoll-Paque (GE bioscience) to separate PBMCs from human peripheral blood, and anti-CD3/CD28 magnetic beads were added to activate them in vitro to obtain T cells. The above lentiviruses PRRL-2F1-28Z, PRRL-2F1-BBZ, PRRL-2B1-28Z, PRRL-2B1-BBZ were used to infect T cells to obtain 2F1-28Z CAR-T, 2F1-BBZ CAR-T, 2B1-28Z CAR-T, 2B1-BBZ CAR-T cells targeting CD94.

2. Preparation of CD94-Targeting TRAC and B2M Double-Negative CAR-T Cells

(1) Preparation of Gene Knockout Cells

After 2F1-28Z CAR-T, 2F1-BBZ CAR-T, 2B1-28Z CAR-T, and 2B1-BBZ CAR-T cells were expanded in vitro for 48 hours, the cell density was adjusted to 2*10\hat{ }7/mL. TRAC and B2M dual gene knockout was performed on CAR-T cells. Cas 9 enzyme (purchased from NEB) and sgRNA were incubated at room temperature at a ratio of 1:4 for 10 minutes to obtain RNP complex. The final concentration of Cas 9 enzyme was 2 μM. Wherein, the nucleic acid sequence of TRAC-gRNA (gRNA used for targeted knockout of TRAC) is shown in SEQ ID NO: 47, and the nucleic acid sequence of B2M-gRNA (gRNA used for targeted knockout of B2M) is shown as SEQ ID NO: 55, and gRNA sequences targeting TRAC and B2M were synthesized in vitro according to the reagent instructions (GeneArt™ Precision gRNA Synthesis Kit, Thermo Tisher). 2*10\hat{ }6 CAR-T cells were mixed with RNP complexes, and the RNP complexes comprising TRAC-gRNA and B2M-gRNA were introduced into CAR-T cells using a maxcyte electroporator. On the 4th day after electroporation, flow cytometry was used to detect the knockout of TTRAC and B2M genes.

According to the above method, TRAC single-gene knockout, TRAC and B2M double-gene knockout were performed on UTD cells (T cells not transfected with virus), respectively.

(2) Screening of TRAC-Negative Cells or TRAC/B2M Double-Negative Cells

TRAC single-knockout UTD cells, B2M and TRAC double-knockout CAR-T cells, and B2M and TRAC double-knockout UTD cells were expanded in vitro, and the cell density was adjusted to 1*10\hat{ }7/mL on the 4th day after electroporation, the cells were labeled with anti-PE-HLA-ABC antibody (Invitrogen) and PE-B2M antibody (Invitrogen), and the labeled cells were sorted by anti-PE magnetic beads on a sorting column, and TRAC-negative cells and TRAC and B2M double-negative cells were collected (the sorting kit was purchased from Miltenyi), that is, more than 99% of TRAC-deleted TRAC−/− UTD cells, TRAC and B2M double-deleted U-2F1-28Z CAR-T cells, U-2F1-BBZ CAR-T cells, U-2B1-28Z CAR-T cells, U-2B1-BBZ CAR-T cells, TRAC and B2M double-deleted UTD cells (U-UTD) were obtained.

CAR-T cells were labeled with Biotin-labeled goat anti-human Fab antibody (Jackson), and then labeled with PE-Streptavidin secondary antibody, and the CAR expression of U-2F1-28Z CAR-T cells, U-2F1-BBZ CAR-T cells, U-2B1-28Z CAR-T cells, and U-2B1-BBZ CAR-T cells were detected by flow cytometry, after enrichment, the proportion of CAR-positive T cells reached more than 95%.

### Example 5. Detection of the Resistance Function of CAR-T Cells Targeting CD94 Against Resting NK Cells

Density gradient centrifugation was performed with Ficoll-Paque (GE bioscience) to separate PBMCs from the peripheral blood of donors, and NK cells in PBMC cells were separated with NK cell isolation kit (purchased from Miltenyi), and the cell density was adjusted to 1*10\hat{ }6/ml. U-UTD cells and TRAC−/-UTD cells were selected as negative and positive controls, respectively, and the cell concentration was adjusted to 1*10\hat{ }6/ml. According to the ratio of NK cells to T cells 1:1, NK cells were mixed with U-UTD, TRAC−/− UTD, U-2F1-28Z CAR-T, U-2F1-BBZ CAR-T, U-2B1-28Z CAR-T cells respectively, and placed in a 24-well plate, and incubated in an incubator for 0 hr, 24 hr and 48 hr, respectively. In the NK cell+TRAC−/− UTD group, NK cells were labeled with APC-CD56 (Invitrogen) antibody, and in the other mixed culture groups, NK cells were labeled with APC-HLA-ABC antibody (Invitrogen). Changes in the proportion and number of T cells and NK cells co-incubated at different time points were detected separately.

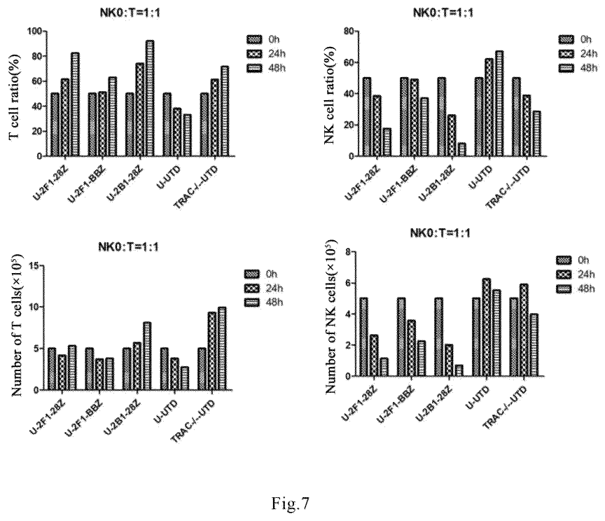

The experimental results were shown in FIG. 7, the proportion and number of U-UTD cells gradually decreased; while the proportion of U-2F1-28Z CAR-T, U-2F1-BBZ CAR-T, U-2B1-28Z CAR-T cells gradually increased, the number of cells was more than the number of T cells in U-UTD+NK cell co-culture. This suggests that U-2F1-28Z CAR-T cells, U-2F1-BBZ CAR-T cells, and U-2B1-28Z CAR-T cells can all effectively resist the killing of resting NK cells, thereby maintaining a high survival rate.

The above results indicate that expressing a CAR targeting CD94 can significantly improve the resistance of T cells to resting NK cells, thereby maintain a high survival rate of T cells.

### Example 6. Detection of the Resistance Function of CAR-T Cells Targeting CD94 Against Activated NK Cells

PBMCs were isolated from the peripheral blood of donors using Ficoll-Paque (GE bioscience) separation medium, and NK cells were isolated using NK cell isolation kit (Miltenyi). NK cells were cultured with NK cell culture medium supplemented with IL-2 (500 U/ml) and IL-15 (150 U/ml) to obtain activated NK cells.

U-UTD cells and TRAC−/-UTD cells were taken as negative and positive controls, respectively. All cell densities were adjusted to a cell density of 1*10\hat{ }6/ml. The activated NK cells and T cells were inoculated at a ratio of 1:1, and the activated NK cells were mixed with U-UTD, TRAC−/− UTD, U-2F1-28Z CAR-T, U-2F1-BBZ CAR-T, U-2B1-28Z CAR-T cells respectively, placed in a 24-well plate and incubated in an incubator for 0 hr, 24 hr and 48 hr, respectively. The NK cells+TRAC−/−UTD group was labeled with APC-CD56 (Invitrogen) antibody, and the NK cells in other mixed culture groups were labeled with APC-HLA-ABC antibody (Invitrogen). Changes in the proportion and number of T cells and NK cells at different time points were detected separately.

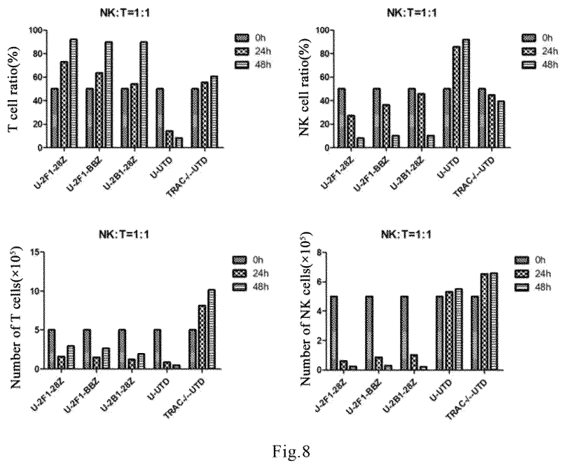

The experimental results are shown in FIG. 8. With the prolongation of incubation time, the proportion of U-UTD cells decreased significantly, while U-2F1-28Z CAR-T, U-2F1-BBZ CAR-T, U-2B1-28Z CAR-T T cells all showed the same changing trend as TRAC−/−UTD, and significant increase was detected at 24 hr and 48 hr. Among them, TRAC−/−UTD cells accounted for about 55.4%, U-2F1-BBZ CAR-T cells accounted for about 63.6%, U-2F1-28Z CAR-T cells accounted for about 73.9%, U-2B1-28Z CAR-T cells accounted for about 54.3% after 24 hr incubation; after 48 hr incubation, TRAC−/−UTD cells accounted for about 60.7%, U-2F1-BBZ CAR-T cells accounted for about 89.9%, U-2F1-28Z CAR-T cells accounted for about 92.1%, and U-2B1-28Z CAR-T cells accounted for about 89.7%. This suggests that U-2F1-28Z CAR-T cells, U-2F1-BBZ CAR-T cells, and U-2B1-28Z CAR-T cells can all effectively resist activated NK cells, thereby maintain a high survival rate.

The above results show that expressing a CAR targeting CD94 can significantly improve the resistance of T cells to activated NK cells, thereby maintain a high survival rate of T cells.

### Example 7. Detection of Resistance Function of CAR-T Cells Targeting CD94 Against PBMC Cells

To determine that UCAR-T cells targeting CD94 can counteract NK cells in donor PBMC cells, we further co-cultured U-2F1-28Z CAR-T cells, U-2F1-BBZ CAR-T cells, U-2B1-28Z CAR-T cells, U-2B1-BBZ CAR-T cells with donor PBMC cells.

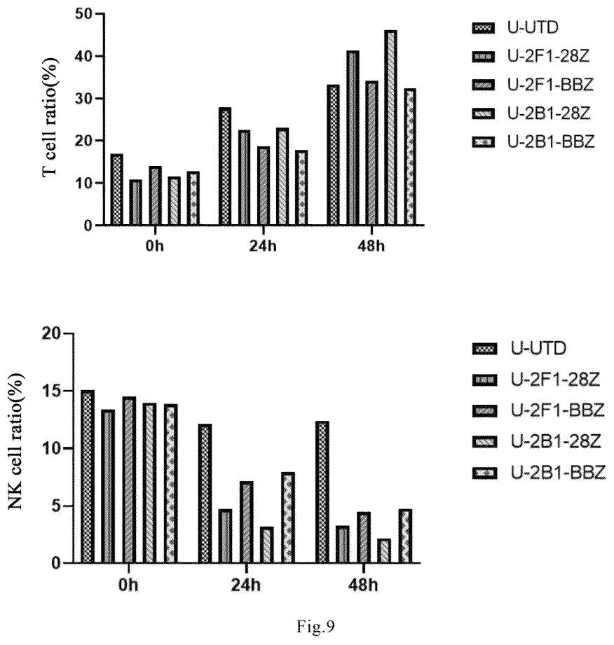

5×10⁵ of U-2F1-28Z CAR-T, U-2F1-BBZ CAR-T, U-2B1-28Z CAR-T, U-2B1-BBZ CAR-T and U-UTD cells were taken respectively and co-cultured with 5×10⁶ donor PBMC cells for 0 h, 24 h and 48 h. The proportion of NK cells in co-culture was detected using PE-anti-HLA-ABC antibody and APC-anti CD56 antibody (FIG. 9).

With the prolongation of co-culture time, the proportion of T cells in the UCAR-T cells co-culture group gradually increased, and the proportion of T cells in the U-2F1-28Z and U-2B1-28Z groups increased more significantly; it was also observed that the proportion of NK cells in the UCAR-T co-culture group decreased gradually, while the proportion of NK cells in the U-UTD co-culture group was not decreased significantly.

Therefore, CAR-T cells targeting CD94 can resist donor PBMC cells, and CAR-T cells with 28Z were more resistant.

### Example 8. Screening of CD94 gRNA

Since part of the T cells express CD94, in order to avoid self-killing by CAR-T cells targeting CD94, the CD94 gene of CAR-T cells was further knocked out on the basis of TRAC/B2M double knockout.

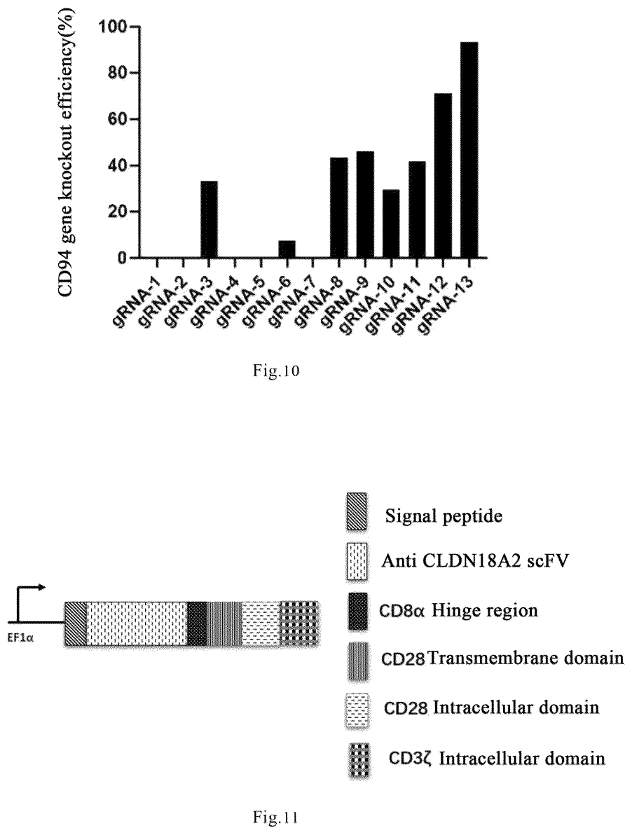

For the CD94 gene, we screened 13 pairs of gRNAs 1-13 (SEQ ID NOs: 59-71) targeting the CD94 gene. And gRNA sequences targeting CD94 were synthesized in vitro following the reagent instructions (GeneArt Precision gRNA Synthesis Kit, Thermo Tisher). NK cells were activated by electroporation using a Maxcyte electroporator. The electroporation system was 1 μM Cas9 (Kactus Biosystems)+4 μM CD94 gRNA, and 2×10⁶ NK cells (50 μl). On the 4th day after electroporation, PE-CD94 antibody was used to detect CD94 gRNA knockout efficiency (FIG. 10).

The results showed that CD94-gRNA-13 had the highest knockout efficiency on CD94 gene, reaching 93%. gRNA3, 6, 8, 9, 10, 11, 12 could also achieve knockout of cd94.

T cells or CAR-T cells on the 5th day of activation were taken, and TRAC, B2M and CD94 were simultaneously triply knocked out by Maxcyte electroporation. The electroporation system was 2 μM Cas9 (Kactus Biosystems)+2 μM TRAC gRNA+2 μM B2M gRNA+4 μM CD94 gRNA, wherein the nucleic acid sequence of TRAC gRNA was shown in SEQ ID NO: 47, the nucleic acid sequence of B2M gRNA was shown in SEQ ID NO: 55, and the nucleic acid sequence of CD94 gRNA was shown in SEQ ID NO: 71. TRAC, B2M and CD94 triple knockout U-UTD-CD94KO cells, U-2F1-28Z CAR-T-CD94KO cells, U-2F1-BBZ CAR-T-CD94 KO cells, U-2B1-28Z CAR-T-CD94 KO cells, and U-2B1-BBZ CAR-T-CD94 KO cells were obtained.

### Example 9: Knockout of CD94 Gene does not Affect the Killing Activity of CAR-T Cells In Vitro

The effect of knockout of endogenous CD94 on target cell killing activity of CAR-T cells was further examined.

Taking the Claudin18.2 CAR-T targeting tumor antigens as an example, we compared the in vitro killing activity of CLDN18A2-UCAR-T and CLDN18A2-UCAR-T-CD94 KO cells on target cells.

1. Construction of CLDN18A2-CAR-T, CLDN18A2CAR-T-CD94 KO Cells

Construction of lentiviral plasmid PRRLsin-CLDN18A2-CAR expressing CLDN18A2 chimeric antigen receptor (FIG. 11), comprising single chain antibody of CLDN18A2 (SEQ ID NO: 72), CD8α signal peptide (SEQ ID NO: 29), CD8α hinge region (SEQ ID NO:31), CD28 transmembrane domain (SEQ ID NO:35), CD28 intracellular domain (SEQ ID NO:37), CD3 (SEQ ID NO:41).

According to the method described in Example 4, CLDN18A2 CAR-T cells and CLDN18A2 UCAR-T cells with knockout of TRAC and B2M were prepared. Cells that were not transfected with virus were named UTD cells. CLDN18A2 CAR-T-CD94 KO cells and CLDN18A2 UCAR-T-CD94 KO cells were obtained by knocking out the endogenous CD94 of CLDN18A2 CAR-T cells and CLDN18A2 UCAR-T cells according to the method described in Example 8, respectively. The nucleic acid sequence of TRAC gRNA is shown in SEQ ID NO: 47, the nucleic acid sequence of B2M gRNA is shown in SEQ ID NO: 55, and the nucleic acid sequence of CD94 gRNA is shown in SEQ ID NO: 71.

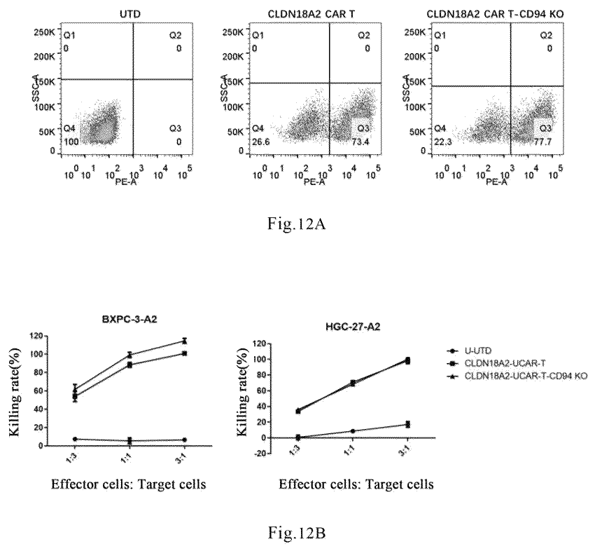

Anti-CLDN18A2scFv antibody was used as the primary antibody, and anti-biotin PE antibody was used as the secondary antibody for flow cytometry. The results are shown in FIG. 12A. The CAR-positive rate of CLDN18A2 CAR-T cells was approximately 73.4%. The CAR-positive rate of CLDN18A2 CAR-T-CD94 KO cells was approximately 77.7%.

2. Construction of BXPC-3-A2 and HGC-27-A2 Cell Lines

Using conventional molecular biology techniques, human CLDN18A2 (SEQ ID NO: 75) was transfected into pancreatic cancer cells BXPC-3 (ATCC, USA) and gastric cancer cells HGC-27 (Cell Bank of Chinese Academy of Sciences) that do not express endogenous CLDN18A2, respectively, by lentivirus. Positive single clones were selected by limited dilution method, and BXPC-3-A2 and HGC-27-A2 stably transfected cell lines expressing CLDN18A2 were constructed.

3. Detection of In Vitro Killing Activity of CLDN18A2-UCAR-T and CLDN18A2-UCAR-T-CD94 KO Cells to Target Cells

The killing of CLDN18A2-UCAR-T and CLDN18A2-UCAR-T-CD94 KO cells to HGC-27-A2 and BXPC-3-A2 cells was detected by xCELLigence RTCA Instrument (Agilent). For the specific method, please refer to the instruction manual of xCELLigence RTCA Instrument.

Target cells: 100 μL of 1×10 5/mL of BXPC-3-A2 cells, HGC-27-A2 cells were inoculated to the corresponding 96-well electrode plate E-Plate 96, placed on the xCELLigence RTCA Instrument.

24 h after inoculation of target cells, effector cells were inoculated at a ratio 1:1 of effector to target: U-UTD cells, CLDN18A2-UCAR-T cells, and CLDN18A2-UCAR-T-CD94 KO cells.

Two duplicate wells were set in each group, and the effects of CLDN18A2-UCAR-T and CLDN18A2-UCAR-T-CD94 KO cells on tumor cell proliferation were continuously observed.

The settings of each experimental group and each control group were as follows:

Experimental group: each target cell+different CAR-T cells;

Control group: each target cell.

The calculation formula is: % cytotoxicity=(1−NCI experimental group/NCI control group)*100. (NCI: Normalized Cell Index).

The results are shown in FIG. 12B, knocking out the endogenous CD94 gene did not affect the killing of target cells by CAR-T cells.

### Example 10. Knockout of CD94 Gene does not Affect the Therapeutic Effect of CAR-T Cells on Tumors

In order to determine the effect of knockout of CD94 gene on the anti-tumor effect of CAR-T cells in vivo, we compared the therapeutic effects of BCMA-CAR-T cells and BCMA-CAR-T CD94 KO cells on PRMI8226 xenograft tumors, using CAR-T cells targeting tumor antigen BCMA as an example.

(1) Construction of BCMA-CAR-T and BCMA-CAR-T-CD94 KO Cells

Using conventional molecular biology methods in the field, using PRRLsin as a vector, a lentiviral plasmid PRRLsin-BCMA-CAR targeting BCMA chimeric antigen receptor was constructed (FIG. 13), comprising a single-chain antibody of BCMA (SEQ ID NO: 77), CD8α signal peptide (SEQ ID NO:29), CD8α hinge region (SEQ ID NO:31), CD8 transmembrane domain (SEQ ID NO:33), CD137 intracellular domain (SEQ ID NO:39), and CD3 (SEQ ID NO: 41).

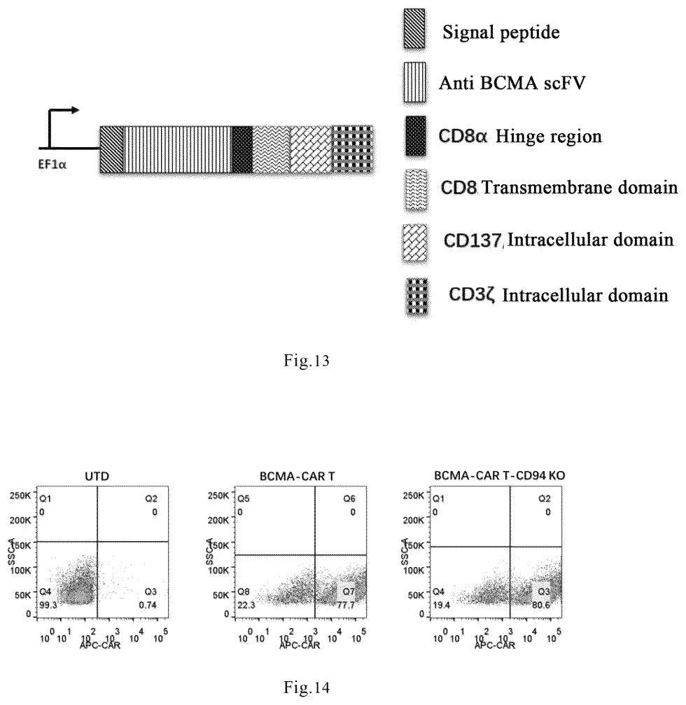

BCMA-CAR-T and BCMA-CAR-T-CD94 KO cells were prepared according to Example 9. The electroporation system was 1 μM Cas9 (Kactus Biosystems)+4 μM CD94 sgRNA, wherein the nucleic acid sequence of CD94 gRNA is shown in SEQ ID NO: 71. On the 9th day of culture, the anti-BCMA scFv antibody was used as the primary antibody, and the anti-biotin APC antibody was used as the secondary antibody for flow cytometry. The results are shown in FIG. 14. The positive rate of BCMA CAR-T cells was about 77.7%. The positive rate of BCMA-CAR-T− CD94 KO cells was about 80.6%.

(2) Therapeutic Effect of BCMA-CAR-T and BCMA-CAR-T-CD94 KO Cells on PRMI8226 Xenograft Tumor

PRMI8226 xenograft tumor were subcutaneously inoculated in immunodeficient mice NPG mice (marked as DO), 5×10⁶ cells were inoculated per mouse, the tumor volume was measured and the mice were grouped on the 11th day, and the xenograft tumor volume was about 180 mm³, divided into 3 groups (UTD, BCMA-CAR-T, BCMA-CAR-T-CD94 KO), 5 mice in each group; 6×10⁵ UTD, BCMA-CAR-T, BCMA-CAR-T-CD94 KO cells were administered through the tail vein. After the injection, the body weight was measured twice a week (including the day of group administration and euthanasia), and the long and short diameters of the tumor were measured and recorded with a vernier caliper, tumor volume was calculated, and the tumor growth curve was drawn according to the tumor volume. The differences in tumor growth curves between groups were compared (tumor volume: V=½×long diameter×short diameter 2) (FIG. 15).

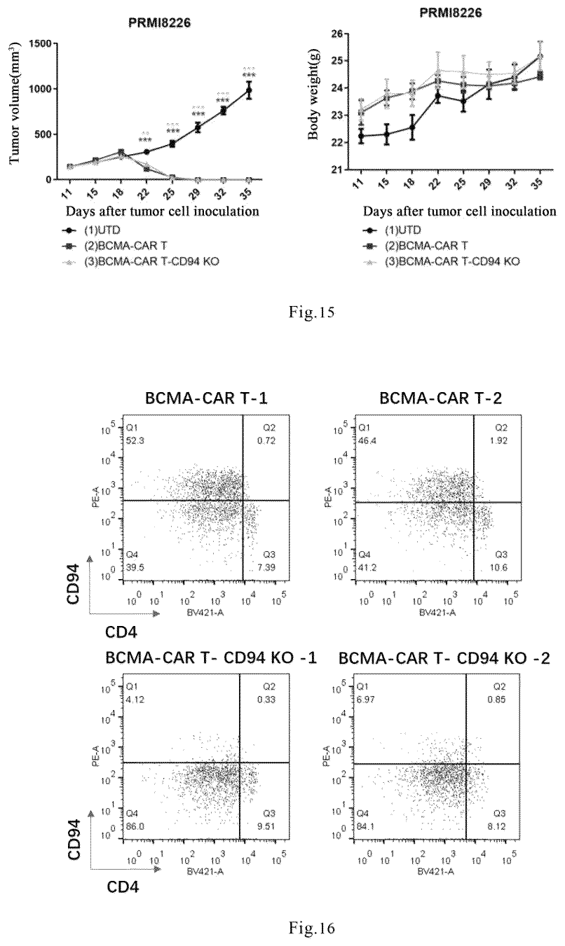

FIG. 15 shows that the two kinds of CAR-T cells have the same therapeutic effect. On the 14th day (D25) of CAR-T treatment, 50 μl of peripheral blood was collected from two mice in each of BCMA-CAR-T and BCMA-CAR-T-CD94 KO cell treatment groups. the expression of CD94 protein in T cells in vivo was detected by APC-CD3 antibody+PE-CD94 antibody+BV421-CD4 antibody (FIG. 16). The results showed that 57% and 53% of the CD8+ T cells (CD3+CD4−T) of the two mice in the BCMA-CAR-T treatment group expressed CD94, respectively, and the proportion of CD94+CD8 T cells in the BCMA-CAR-T-CD94 KO treatment group were 4.6% and 7.6%, respectively, indicating that knocking out the CD94 gene did not affect the therapeutic effect of CAR-T cells on xenograft tumors.

### Example 11. Detection of Anti-Tumor Effect of CAR-T Cells Targeting Tumor Antigens in Combination with CAR-T Cells Targeting CD94 in Presence of NK Cells

In order to investigate whether immune cells targeting CD94 affect the anti-tumor effect of universal CAR-T cells in presence of NK cells, taking the CAR-T targeting the tumor antigen BCMA as an example, the therapeutic effects of BCMA-UCAR-T-CD94 KO, BCMA-UCAR-T-CD94 KO+NK and BCMA-UCAR-T-CD94 KO+U-2F1-28Z-CD94 KO+NK on PRMI8226 xenograft tumor were compared.

BCMA-UCAR-T-CD94 KO cells and U-2F1-28Z CAR-T-CD94 KO cells were prepared according to the method described in Example 8. The electroporation system was 2 μM Cas9 enzyme (Kactus Biosystems)+2 μM B2M gRNA+2 μM TRAC gRNA+4 μM CD94 gRNA. Wherein the nucleic acid sequence of TRAC gRNA is shown in SEQ ID NO: 47, the nucleic acid sequence of B2M gRNA is shown in SEQ ID NO: 55, and the nucleic acid sequence of CD94 gRNA is shown in SEQ ID NO: 71. See Example 4 for other operations. The positive rate of CAR-T cells is shown in FIG. 17, wherein the positive rate of BCMA-UCAR-T-CD94 KO cells was 88.2%, and the positive rate of U-2F1-28Z CAR-T-CD94 KO cells was 64.1%. Activated NK cells were prepared according to Example 6.

On day 0, NPG mice were subcutaneously inoculated with PRMI8226 xenograft tumors, and each mouse was inoculated with 5×10⁶ cells. On day 20, the tumor volume was measured and grouped. The xenograft tumor volume was about 290 mm³, the mice were divided into 5 groups, 4 in each group, 5×10⁵ U-UTD-CD94 KO (group 1), 5×10⁵ BCMA-UCAR-T-CD94 KO (group 2), 5×10⁵ BCMA-UCAR-T-CD94 KO (group 3), 5×10⁵ BCMA-UCAR-T-CD94 KO+1×10⁶ U-2F1-28Z CAR-T-CD94 KO (group 4) were injected into the tail vein of the mice. Among them, the 1st, 3rd, and 4th treatment groups were given 1×10⁶ activated NK cells via the tail vein on the 19th, 21st, 25th, and 27th day. After injection, the body weight was measured twice a week (including the day of group administration and euthanasia), and the long and short diameters of the tumor were measured and recorded with a vernier caliper, the tumor volume was calculated, and the tumor growth curve was drawn according to the tumor volume. The differences in tumor growth curves between groups were compared (tumor volume: V=½×long diameter×short diameter 2).

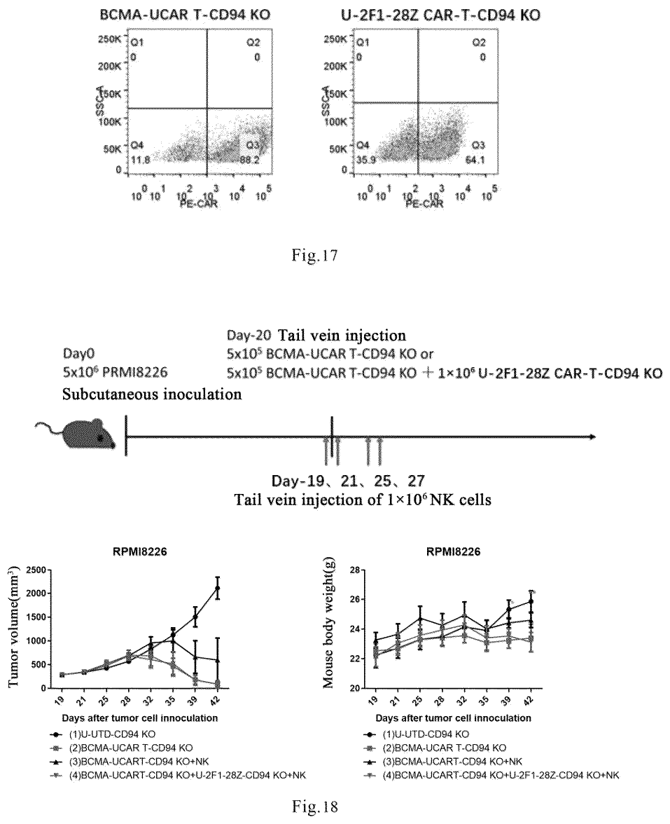

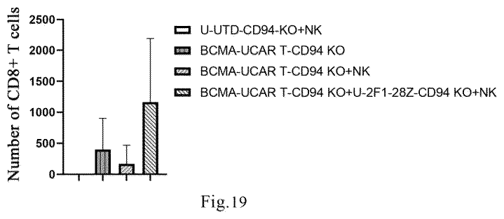

The results are shown in FIG. 18, the therapeutic effect of BCMA-UCAR-T-CD94 KO+U-2F1-28Z CAR-T-CD94 KO+NK on PRMI8226 xenograft tumor was better than that of BCMA-UCAR-T-CD94 KO+NK, and was basically consistent with the therapeutic effect of BCMA-UCAR-T-CD94 KO alone.

After 14 days of CAR-T treatment, the peripheral blood of mice in each group was collected. The survival of CD4+ T cells and CD8+ T cells in mouse peripheral blood was detected by FITC-CD4/PE-CD8 antibody (FIG. 19). When detecting the survival of T cells in peripheral blood, only CD8+ T cells were detected. The survival of CD8+ T cells in peripheral blood of BCMA-UCAR-T-CD94 KO+U-2F1-28Z CAR-T-CD94 KO+NK treatment group was significantly higher than that of other groups. Therefore, the combination of CAR-T cells targeting CD94 can significantly resist the killing effect of NK cells on tumor antigen-targeting CAR-T or Universal CAR-T cells, thereby improving antitumor activity of the tumor antigen-targeting CAR-T or universal CAR-T cells.

All documents mentioned in this application are incorporated herein by reference as if each document were individually incorporated by reference. In addition, it should be understood that after reading the above teaching content of the present application, those skilled in the art can make various changes or modifications to the present application, and these equivalent forms also fall within the scope defined by the appended claims of the present application.

1-48. (canceled)

49. A genetically engineered cell, expressing a first protein recognizing CD94, preferably, wherein the first protein comprises an antibody capable of recognizing CD94, transmembrane domain, and intracellular signal domain, preferably, the amino acid sequence of CD94 is shown in SEQ ID NO:1.

50. The cell of claim 49, wherein the first protein comprises a chimeric antigen receptor (CAR), a chimeric T cell receptor, a T cell antigen coupler (TAC), or a combination thereof,
preferably, the first protein is a CAR, comprising:
(i) an antibody recognizing CD94, a transmembrane domain of CD8/CD28, and CD3ζ;
(ii) an antibody recognizing CD94, a transmembrane domain of CD8/CD28, an intracellular signaling domain of CD137, and CD3ζ,
(iii) an antibody recognizing CD94, a transmembrane domain of CD8/CD28, an intracellular signaling domain of CD28, and CD3ζ; and/or
(iv) an antibody recognizing CD94, a transmembrane domain of CD8/CD28, an intracellular signaling domain of CD28, CD137, and CD3ζ.

51. The cell of claim 49, comprising: knockout of the gene encoding CD94 protein and/or low or no expression of an endogenous CD94 molecule.

52. The cell of claim 49, wherein the cell is selected from a T cell, an NK cell, a cytotoxic T cell, an NKT cell, a DNT cell, an NK92 cell, a macrophage, a CIK cell, and a stem cell-derived immune effector cell, or a combination thereof, from autologous or allogeneic.

53. The cell of claim 49, wherein the endogenous MHC molecule B2M and the endogenous TCR are knocked out using CRISPR/Cas9 technology.

54. The cell of claim 49, wherein the first protein also recognizes a tumor and/or a pathogen; preferably, the tumor expresses BCMA, CD19, GPC3, CLDN18A2, EGFR, CD20, CD22, CD38, GPRC5D, B7H3, Claudin 6, FAP, Mesothelin, NKG2D ligand or a combination thereof.

55. The cell of claim 49, further expressing a second protein that targets and recognizes a tumor antigen and/or a pathogen antigen, a chemokine, a chemokine receptor, a cytokine, siRNA for reducing PD-1 expression, a protein for blocking the binding of PD-L1 to PD-1, a safety switch, or a combination thereof.

56. The cell of claim 49, wherein, the cell is administered in combination with another cell expressing a second protein that recognizes an antigen different from the first protein, wherein the second protein comprises extracellular domain recognizing a tumor and/or a pathogen, transmembrane domain, and intracellular signal domain.

57. The cell of claim 56, wherein the tumor and/or pathogen expresses CLDN18A2, BCMA, CD19, GPC3, EGFR, CD20, CD22, CD38, GPRC5D, B7H3, Claudin 6, FAP, Mesothelin, NKG2D ligand or a combination thereof.

58. The cell of claim 57, wherein the second protein comprises the sequence shown in SEQ ID NO: 74, 79, 88, 89, 90, 99 or 100.

59. The cell of claim 56, wherein the cell expressing the second protein comprises:
(i) endogenous CD94 molecule is knocked out using CRISPR/Cas9 technology;
(ii) TCR and/or MHC molecule B2M is knocked out using CRISPR/Cas9 technology; or
(iii) CD94, TCR and MHC molecule B2M is knocked out using CRISPR/Cas9 technology.

60. The cell of claim 56, wherein the another cell expressing the second protein is selected from a T cell, an NK cell, a cytotoxic T cell, an NKT cell, a DNT cell, an NK92 cell, a macrophage, a CIK cell, and a stem cell-derived immune effector cell, or a combination thereof, from autologous or allogeneic.

61. A method for increasing the persistence and/or transplantation survival rate of a first immune cell in the presence of a host second immune cell, comprising:
a) providing the first immune cell;
b) optionally, modifying the first immune cell by reducing or inhibiting the expression, activity and/or signal transduction of at least one endogenous gene encoding a polypeptide involved in response to autologous and non-autologous antigen recognition;
c) modifying the first immune cell by a polynucleotide encoding a first protein targeting CD94;
wherein the first protein comprises the antibody recognizing CD94, transmembrane domain, and intracellular signal domain.

62. The method of claim 61, wherein, the step b) comprises:
(i) knockout of CD94 molecule using CRISPR/Cas9 technology;
(ii) knockout of TCR and/or MEW molecule B2M using CRISPR/Cas9 technology; or
(iii) knockout of CD94, TCR and MEW molecule B2M using CRISPR/Cas9 technology.

63. The method of claim 61 wherein, the step b) comprises:
the gRNA used is selected from the sequence(s) shown in SEQ ID NO(s): 71, 47, and/or 55.

64. The method of claim 63, wherein, the first protein comprises a chimeric antigen receptor (CAR), a chimeric T cell receptor, a T cell antigen coupler (TAC), or a combination thereof.

65. The method of claim 61, wherein, the first immune cell is selected from an autologous or an allogeneic T cell, an NK cell, a cytotoxic T cell, an NKT cell, a DNT cell, an NK92 cell, a macrophage, a CIK cell, and a stem cell-derived immune effector cell, or a combination thereof, from autologous or allogeneic; wherein the host second immune cell is NK cells.

66. An antibody recognizing CD94, wherein, the antibody is selected from the group consisting of:
(1) an antibody, comprising HCDR1 shown in SEQ ID NO: 11, HCDR2 shown in SEQ ID NO: 12 and HCDR3 shown in SEQ ID NO: 13; LCDR1 shown in SEQ ID NO: 14, LCDR2 shown in SEQ ID NO: 15 and LCDR3 shown in SEQ ID NO: 16; or
(2) an antibody, comprising HCDR1 shown in SEQ ID NO: 11, HCDR2 shown in SEQ ID NO: 12 and HCDR3 shown in SEQ ID NO: 17; LCDR1 shown in SEQ ID NO: 14, LCDR2 shown in SEQ ID NO: 15 and LCDR3 shown in SEQ ID NO: 18; or
(3) an antibody, the heavy chain variable region of the antibody has the amino acid sequence shown in SEQ ID NO: 19, and the light chain variable region of the antibody has the amino acid sequence shown in SEQ ID NO: 21; or
(4) an antibody, the heavy chain variable region of the antibody has the amino acid sequence shown in SEQ ID NO: 24, and the light chain variable region of the antibody has the amino acid sequence shown in SEQ ID NO: 26; (5) an antibody, scFv of the antibody has the amino acid sequence shown in SEQ ID NO: 23, 28.

67. The antibody according to claim 66, wherein, the antibody is a whole antibody, scFv, a single domain antibody, a Fab fragment, a Fab′ fragment, a Fv fragment, a F(ab′)2 fragment, a Fd fragment, a dAb fragment or a multifunctional antibody.

68. The cell of claim 49, wherein, the first protein comprises the sequence shown in SEQ ID NO: 11, 12, 13, 14, 15, 16, 17, 18, 19, 21, 23, 24, 26, 28, 43, 44, 45 or 46.
